SEQUENCE LISTING (67 sequences)

SEQ ID NO: 1 - protein, 406 aa, Homo sapiens
  A synthetic renin inhibitor sequence
MDGWRRMPRWGLLLLLWGSCTFGLPTDTTTFKRIFLKRMPSIRESLKERGVDMARLGPEWSQPMKRLTLGNTTSSVILTNYMDTQYYGEIGIGTPPQTFKVVFDTGSSNVWVPSSKCSRLYTACVYHKLFDASDSSSYKHNGTELTLRYSTGTVSGFLSQDIITVGGITVTQMFGEVTEMPALPFMLAEFDGVVGMGFIEQAIGRVTPIFDNIISQGVLKEDVFSFYYNRDSENSQSLGGQIVLGGSDPQHYEGNFHYINLIKTGVWQIQMKGVSVGSSTLLCEDGCLALVDTGASYISGSTSSIEKLMEALGAKKRLFDYVVKCNEGPTLPDISFHLGGKEYTLTSADYVFQESYSSKKLCTLAIHAMDIPPPTGPTWALGATFIRKFYTEFDRRNNRIGFALAR

SEQ ID NO: 2 - protein, 406 aa, Homo sapiens; identical to SEQ ID NO: 1

SEQ ID NO: 3 - protein, 5 aa, Artificial Sequence
  A synthetic renin inhibitor sequence
XXXXX

SEQ ID NO: 4 - protein, 5 aa, Artificial Sequence
  A synthetic peptide sequence
FFVYK

SEQ ID NO: 5 - protein, 5 aa, Artificial Sequence
  A synthetic peptide sequence
YFVYK

SEQ ID NO: 6 - protein, 5 aa, Artificial Sequence
  A synthetic peptide sequence
XFVYK

SEQ ID NO: 7 - protein, 5 aa, Artificial Sequence
  A synthetic peptide sequence
FYVYK

SEQ ID NO: 8 - protein, 5 aa, Artificial Sequence
  A synthetic peptide sequence
FXVYK

SEQ ID NO: 9 - protein, 5 aa, Artificial Sequence
  A synthetic peptide sequence
FXVYK

SEQ ID NO: 10 - protein, 5 aa, Artificial Sequence
  A synthetic peptide sequence
FXVYK

SEQ ID NO: 11 - protein, 5 aa, Artificial Sequence
  A synthetic peptide sequence
FFTYK

SEQ ID NO: 12 - protein, 5 aa, Artificial Sequence
  A synthetic peptide sequence
FFXYK

SEQ ID NO: 13 - protein, 5 aa, Artificial Sequence
  A synthetic peptide sequence
FFVFK

SEQ ID NO: 14 - protein, 5 aa, Artificial Sequence
  A synthetic peptide sequence
FFVXK

SEQ ID NO: 15 - protein, 8 aa, Artificial Sequence
  A synthetic renin inhibitor sequence
XHPFHXLF

SEQ ID NO: 16 - protein, 6 aa, Artificial Sequence
  A synthetic renin inhibitor sequence
CHPFHC

SEQ ID NO: 17 - protein, 6 aa, Artificial Sequence
  A synthetic renin inhibitor sequence
KCHPFH

SEQ ID NO: 18 - protein, 6 aa, Artificial Sequence
  A synthetic renin inhibitor sequence
CHPFHC

SEQ ID NO: 19 - protein, 6 aa, Artificial Sequence
  A synthetic renin inhibitor sequence
KCHPFH

SEQ ID NO: 20 - protein, 10 aa, Artificial Sequence
  A synthetic renin inhibitor sequence
PHPFHFFVYK

SEQ ID NO: 21 - protein, 5 aa, Artificial Sequence
  A synthetic renin inhibitor sequence
HPFHL

SEQ ID NO: 22 - protein, 4 aa, Artificial Sequence
  A synthetic renin inhibitor sequence
PFHL

SEQ ID NO: 23 - protein, 8 aa, Artificial Sequence
  A synthetic renin inhibitor sequence
HPFHXLVY

SEQ ID NO: 24 - protein, 14 aa, Artificial Sequence
  A synthetic renin inhibitor sequence
DRVYIHPFHLLVYS

SEQ ID NO: 25 - protein, 9 aa, Artificial Sequence
  A synthetic renin inhibitor sequence
PHPFHFFVY

SEQ ID NO: 26 - protein, 8 aa, Artificial Sequence
  A synthetic renin inhibitor sequence
XPFHLLVY

SEQ ID NO: 27 - protein, 7 aa, Artificial Sequence
  A synthetic renin inhibitor sequence
HPFHXLF

SEQ ID NO: 28 - protein, 10 aa, Artificial Sequence
  A synthetic renin inhibitor sequence
PHPFHLVIHK

SEQ ID NO: 29 - protein, 8 aa, Artificial Sequence
  A synthetic renin inhibitor sequence
XHPFHXIF

SEQ ID NO: 30 - protein, 5 aa, Artificial Sequence
  A synthetic renin inhibitor sequence
FHXAX

SEQ ID NO: 31 - protein, 5 aa, Artificial Sequence
  A synthetic renin inhibitor sequence
LKKMX

SEQ ID NO: 32 - protein, 8 aa, Artificial Sequence
  A synthetic renin inhibitor sequence
RIPLKKMX

SEQ ID NO: 33 - protein, 9 aa, Artificial Sequence
  A synthetic renin inhibitor sequence
ERIPLKKMX

SEQ ID NO: 34 - protein, 5 aa, Artificial Sequence
  A synthetic renin inhibitor sequence
VVXAX

SEQ ID NO: 35 - protein, 7 aa, Artificial Sequence
  A synthetic renin inhibitor sequence
XVVXAXE

SEQ ID NO: 36 - protein, 4 aa, Artificial Sequence
  A synthetic renin inhibitor sequence
HXLF

SEQ ID NO: 37 - protein, 5 aa, Artificial Sequence
  A synthetic renin inhibitor sequence
FHXLF

SEQ ID NO: 38 - protein, 6 aa, Artificial Sequence
  A synthetic renin inhibitor sequence
PFHXLF

SEQ ID NO: 39 - protein, 6 aa, Artificial Sequence
  A synthetic renin inhibitor sequence
XHPFHX

SEQ ID NO: 40 - protein, 7 aa, Artificial Sequence
  A synthetic renin inhibitor sequence
XHPFHXL

SEQ ID NO: 41 - protein, 8 aa, Artificial Sequence
  A synthetic renin inhibitor sequence
XHPFHXLF

SEQ ID NO: 42 - protein, 8 aa, Artificial Sequence
  A synthetic renin inhibitor sequence
XHPFHXAF

SEQ ID NO: 43 - protein, 8 aa, Artificial Sequence
  A synthetic renin inhibitor sequence
XHPFHXVF

SEQ ID NO: 44 - protein, 8 aa, Artificial Sequence
  A synthetic renin inhibitor sequence
XHPFHXIF

SEQ ID NO: 45 - protein, 7 aa, Artificial Sequence
  A synthetic renin inhibitor sequence
HPFHXLY

SEQ ID NO: 46 - protein, 6 aa, Artificial Sequence
  A synthetic renin inhibitor sequence
HPFHXL

SEQ ID NO: 47 - protein, 9 aa, Artificial Sequence
  A synthetic renin inhibitor sequence
XHPFHXLVF

SEQ ID NO: 48 - protein, 4 aa, Artificial Sequence
  A synthetic renin inhibitor sequence
LXVX

SEQ ID NO: 49 - protein, 4 aa, Artificial Sequence
  A synthetic renin inhibitor sequence
LXLX

SEQ ID NO: 50 - protein, 4 aa, Artificial Sequence
  A synthetic renin inhibitor sequence
HXLX

SEQ ID NO: 51 - protein, 9 aa, Artificial Sequence
  A synthetic renin inhibitor sequence
XHPFHLXVF

SEQ ID NO: 52 - protein, 8 aa, Artificial Sequence
  A synthetic renin inhibitor sequence
XHPFHXLF

SEQ ID NO: 53 - DNA, 5979 bp, Homo sapiens
  misc_feature (1)...(5979): n = A, T, G, or C
acaagaagtaactcttataaatgctccagaggccctcagtgacagaggtgatttccaggtggctgggctaacgttaaaggtggttgtactaaagagaggggtttggcctcagggactcacatgtggtggaggtacagcacttttctatttttgcttcctccaccctgggccaggatcttcctcaagagaatgccctcaatccgagaaagcctgaaggaacgaggtgtggacatggccagccttggtcccgagtggagccaacccatgaagaggctgacacttggcaacaccacctcctccgtgatcctcaccaactacatggacgtgagtgcttggctcagcccctcgctccctccctgtctcctttccctcatggacctagggctttctttgctgcaagactcaccctttccaagctgtgtttgacgaaggcgctgagtagctgcaggaaaatggaaaccccgacaggtataggacctcgcctggggcaagtctacacccgagagccaagagtgaagccaggcaagaccccaagcccaaggtcccctgagcccctccagccctctctttttaccccacagacccagtactatggcgagattgggatcgggaccccaccccaaaccttcaaagtcgtctttgacactggttcgtccaatgtttgggtgccctcctccaagtgcagccgtctctacactgcctgtggtgagacctaagacccacactgcctctcctccatccccctgccctactgtgcatgagcaatcctgcccaacacccagctcccatccctcttgccaccaagggagtggcttcctctctgcctctgtgcccactgacatgtaggggagaggggaagatgtctcccgtttttctgatacagccaccaaggttaaaaacaaaaaaaggtccaagaacccctgagnacccaggaggaccagttcccagtcgtcctgagattgagacaggactgaattctcaaacccatcccaggcactcggaactcttccatccctagtcttaatcaacaacctcttactagcacttactctgtgcctggcatacttctctggtgttatcagtggttagtgattactttaaattccttcatttaggacaaaattctcgatgtatgggacacttaggagagcccaagaaacccagtccttgattgatgaagcacatattccaagccccctgaccctagggccactcatccctgcacctaagctaaccagccatacccacaatgcaccctgcctctgagtccccctgtctgggccactcttggacaaacctgagcctctgtccccctgccagtgtatcacaagctcttcgatgcttcggattcctccagctacaagcacaatggaacagaactcaccctccgctattcaacagggacagtcagtggctttctcagccaggacatcatcaccgtaagttgggccgccctaggtcatctgccccggaccccttctgtccccaggcctctcctgacccaccagggcccacacctgcggggaggtacactgcagcccacttggagcctggggagctgaggaacaccctactctgccacatctggtgttgaaagcagcagtacctatgggggagcaagcctgggctacgggctcaccgttgggtggtttgtggatgtttttgcatctaacttgcatgtagggctgtcctgagccccgtggctgcagtcaagtaactcgtcccaagttcaccagctctgactggggctactaccctagactgaaatcctgggtcagagtcaggctattttagggtcaggcatagttttaaggtcacattagttgactctgggactcaggtcaaggctctcttttcttttccatgtggcccatgtctgaccgtttcctcatcctggagtttctcaggccctgctccatcagagttaggggaggggcacacgtggcacctgagaggaaatcagggtgattcctgcctcccttcctttttctgttgaactctgatataaaggaggaagaagggcaagcttgtctgtgctaaagaaacccttcgcccatgataagggtggggccaagacccagtcctgccaggcacgaaagtctggccactggggaggggaggagctcttggacttttcttttgcgcttggcaggaccaccctctcagcctctgctctccgatccctggtcaactctagctctctctgggctccgcagcagagatgtgtattggcacagagtgtgtgcgtgcagggttgaggcaatactcttaccccgatttctgtaccctggagcatgtgtgcccctgggatccctagtgtggatgcccagaccagactccaaccaaggaggggcagtgggcttggtctcctatggtccttcctcccacaggtgggtggaatcacggtgacacagatgtttggagaggtcacggagatgcccgccttacccttcatgctggcccagtttgatggggttgtgggcatgggcttcattgaacaggccattggcagggtcacccctatcttcgacaacatcatctcccaaggggtgctaaaagaggacgtcttctctttctactacaacaggtggggactgggactccaagggctgaggtggggggcaggaggggagaagagatggggactggaaggagagtctgggccagaattgtaaagtgtttgtaacttaggtgacagccaatcaatatctagagctgtactagccaatatggaaggcactattgcaaatttaaacttaacttaaatacagcttaagcatcaattaagcattcaactggctggcctcttagttgtactagccacagctcaatgcctggcagccacggtggctagtaactacagtctagtacagtgcagatagagatatccagcatgacaggacatctatagacagcgccactaaaagaatagaggaggatcagagttcagagaaatctcacagtaaaatggagaggagtctccggtttggtgatagaaagtgaggccttgagaaaaggccaattggcggctctgcattcaggggtggtctttagaagaactgttttagaggaggtgggggcaaggccagatggcaagaagttaagaggtggacgacgtgggtgtcaggaagtggaggtcatgagatgtacgctgccctgggacattcaacagggaagggaatggggggtggcgtgggggggtgagatccagaagcagaagaggaagggtgggtgtttttaaatgctagaggatgctcgagtgatcgcctgtaggtggaggaagaacccaatagaaagaaagagattaaaaatgtggaaagaagaggagctaaatgggggcactggagtttagaggccttgaaagagatgaggaaccagcagataggaagaagccaggttttacagaggagagggctggcctcttcttttatcttgggatgggaaggagggaacatccagagagatactgaagtgttgagagacaggcaggagggaatttgtgctagcatatacacatacgagttccgaatttataaaaacacaagtagtttgcagttgcacaaaataacatatgcacacctacacacccatgcacacatgtgcatgtgaattctggaaaaacacatcacacacacaggcatgccctggagactaggcctacagtagtccctgagccaagtgcagtgaggaggaaaggaaggtgaggggaatcatctccagacggggcaccaggagcctggctccagtcccccacttgttcactcatggactgggtaacttcaggcaagtgacttcgcctcttggtgactccattgcctgaagggcaaagagagtacataacacccaccctgccaaacagcagggtgatgaggctggcatgaaatgaagcttcctttctgctgtctctctttctctgcagagattccgagtaaggagacaaaacccccacatggctgtgaccttccagtattccccgagcacctgacctagaattacacacgccaccggcccaaaactcacatcagcaagtcccagcctccgctagatgccgaagttctctgtctctccttcctgctctctccatgccacctgcccaccccatacccaatagcctccccagggtcccctcccatgcacctgctcaatcagcagcaacccaagagtgaggggtgtccatttgtgtcttgttcacatccactcactgtccttgtacctgctccttttctgtgacctctctggggatgctttttgggggaacagctggactaccctggaacaacctctggttggtcttggggaggggaagaaaggcagagaagcagtatgttctgcatgcttcccaacgacagctccgagcctggctgtctgtcccacattcctctgctctagagccctctgtcctcccctcgacccttgtgcaaccttccccaattgcctgagttgctgggtcctggaggttatgggtttccaagagcttctgatctttcctttaggaattcccaatcgctgggaggacagattgtgctgggaggcagcgacccccagcattacgaagggaatttccactatatcaacctcatcaagactggtgtctggcagattcaaatgaaggggtcagaaatcctcagaccctccccgggctccaaaaaatgctgccgtcactggggttggggagggcgggcgcggactgcattaccatcctgccctctttccaaatgcagccacttcttaagcacagccaccatttgctctctgcctggctctggtccaggctggggcagagagaagggaggggcctgggccggagtggtggaggccgagagtaccttccctcctctactcactgcctcaacagccagccagcgtggcgctccacccacccacccaccactcaggaaggacatgcagcctggcgtgcccatcagccttctgtctgtctgtctgtctgtctgtctctctgtctgactgtggcgctcccccagggtgtctgtggggtcatccaccttgctctgtgaagacggctgcctggcattggtagacaccggtgcatcctacatctcaggttctaccagctgcatagagaagctcatggaggccttgggagccaagaagaggctgtttgatgtaagaagccaaagagggaaggtgctgtgggtgtggggagcggccacctggtatcggctcacaaatcccccaggcaaatgaggccatctcaggccttcgcttgttcacctcacactctccacacatgtggctggtcacccatggggcggggcactgtccccagccctctccagcagagagacccagggccaccagcgcaggactccttgtctgctgagacgtcgttccatactcaagaaggctctctttgccccccaccccagtatgtcgtgaagtgtaacgagggccctacactccccgacatctctttccacctgggaggcaaagaatacacgctcaccagcgcggactatgtatttcaggtgaggttcgagtcggccccctcggtggcagggagaaaggctggacagagaccctcaagagtgacagattacaatgcacagatcatgttagaactgtagttctcaaacttggctgtgcatgtcacctggagagctttggaaaaatccaggtacctgggccacatcccatacctattaaatcagaacctctagaagtgggacctggggttcagtttccccagatgattccaatgtgtggccatgtttgggcatcactatgcctgttccctcatctccattttctcatcaaatactcccaataatcctatgctcctatattcttaccctcttttcataatcaataggcttagagaatttgaataacttgtctaggatcagaagctaaggcaaactgtaagctcctgaaggaagcacgttgcctgatgccctgtttgcctgggatctagcacaggggctaaacataggaatggtgcagtccacgatggggcaaaat

SEQ ID NO: 54 - DNA, 1461 bp, Homo sapiens
agaacctcagtggatctcagagagagccccagactgagggaagcatggatggatggagaaggatgcctcgctggggactgctgctgctgctctggggctcctgtacctttggtctcccgacagacaccaccacctttaaacggatcttcctcaagagaatgccctcaatccgagaaagcctgaaggaacgaggtgtggacatggccaggcttggtcccgagtggagccaacccatgaagaggctgacacttggcaacaccacctcctccgtgatcctcaccaactacatggacacccagtactatggcgagattgggatcgggaccccaccccaaaccttcaaagtcgtctttgacactggttcgtccaatgtttgggtgccctcctccaagtgcagccgtctctacactgcctgtgtgtatcacaagctcttcgatgcttcggattcctccagctacaagcacaatggaacagaactcaccctccgctattcaacagggacagtcagtggctttctcagccaggacatcatcaccgtgggtggaatcacggtgacacagatgtttggagaggtcacggagatgcccgccttacccttcatgctggccgagtttgatggggttgtgggcatgggcttcattgaacaggccattggcagggtcacccctatcttcgacaacatcatctcccaaggggtgctaaaagaggacgtcttctctttctactacaacagagattccgagaattcccaatcgctgggaggacagattgtgctgggaggcagcgacccccagcattacgaagggaatttccactatatcaacctcatcaagactggtgtctggcagattcaaatgaagggggtgtctgtggggtcatccaccttgctctgtgaagacggctgcctggcattggtagacaccggtgcatcctacatctcaggttctaccagctccatagagaagctcatggaggccttgggagccaagaagaggctgtttgattatgtcgtgaagtgtaacgagggccctacactccccgacatctctttccacctgggaggcaaagaatacacgctcaccagcgcggactatgtatttcaggaatcctacagtagtaaaaagctgtgcacactggccatccacgccatggatatcccgccacccactggacccacctgggccctgggggccaccttcatccgaaagttctacacagagtttgatcggcgtaacaaccgcattggcttcgccttggcccgctgaggccctctgccacccaggcaggccctgccttcagccctggcccagagctggaacactctctgagatgcccctctgcctgggcttatgccctcagatggagacattggatgtggagctcctgctggatgcgtgccctgacccctgcaccagcccttccctgctttgaggacaaagagaataaagacttcatgttcac

SEQ ID NO: 55 - DNA, 23 bp, Artificial Sequence
  A synthetic oligonucleotide
aacctcagtggatctcagagaaa

SEQ ID NO: 56 - DNA, 23 bp, Artificial Sequence
  A synthetic oligonucleotide
aagcatggatggatggagaagaa

SEQ ID NO: 57 - DNA, 22 bp, Artificial Sequence
  A synthetic oligonucleotide
aaggatgcctcgctggggacaa

SEQ ID NO: 58 - DNA, 19 bp, Artificial Sequence
  A synthetic siRNA
gagaaaggctggacagaga

SEQ ID NO: 59 - DNA, 19 bp, Artificial Sequence
  A synthetic siRNA
tcaactggctggcctctta

SEQ ID NO: 60 - DNA, 19 bp, Artificial Sequence
  A synthetic siRNA
gtacagcacttttctattt

SEQ ID NO: 61 - DNA, 19 bp, Artificial Sequence
  A synthetic siRNA
gcaaagagagtacataaca

SEQ ID NO: 62 - DNA, 20 bp, Artificial Sequence
  A synthetic primer
tctcagccaggacatcatca

SEQ ID NO: 63 - DNA, 20 bp, Artificial Sequence
  A synthetic primer
agtggaaattcccttcgtaa

SEQ ID NO: 64 - DNA, 21 bp, Artificial Sequence
  A synthetic primer
gctcgtcgtcgacaacggctc

SEQ ID NO: 65 - DNA, 26 bp, Artificial Sequence
  A synthetic primer
gctcttctactgggtctagtacaaac

SEQ ID NO: 66 - DNA, 275 bp, Homo sapiens
tcgctgcctcccagcacaatctgtcctcccagcgattgggaattctcggaatctctgttgtagtagaaagagaagacgtcctcttttagcaccccttgggagatgatgttgtcgaagataggggtgaccctgccaatggcctgttcaatgaagcccatgcccacaaccccatcaaactcggccagcatgaagggtaaggcgggcatctccgtgacctctccaaacatctgtgtcaccgtgattccacccacggtgatgatgtcctggctgagaaa

SEQ ID NO: 67 - protein, 7 aa, Artificial Sequence
  A synthetic renin inhibitor sequence
HPFXLVY

(19) United States
(12) Patent Application Publication
(10) Pub. No.: US 2005/0209141 A1
(43) Pub. Date: Sep. 22, 2005

(54) Mast cell-derived renin

(76) Inventors: Randi B. Silver, New York, NY (US); Roberto Levi, New York, NY (US)

Correspondence Address: Schwegman, Lundberg, Woessner & Kluth, P.A., P.O. Box 2938, Minneapolis, MN, 55402, US

(21) Appl. No.: 10/964,567

(22) Filed: Oct. 13, 2004

Related U.S. Application Data
(60) Provisional application No. 60/512,142, filed on Oct. 17, 2003.

(51) Int. Cl. A61K 38/17; A61K 48/00; A61K 31/401

(52) U.S. Cl. 514/12; 514/44; 514/423

(57) ABSTRACT

The invention relates to the discovery that renin is present in mast cells and can act in a localized manner to initiate and/or exacerbate a number of conditions. Thus, the invention provides methods for treating cardiac, vascular, lung, liver, cervical, intestinal, muscle, pancreatic, brain, kidney, skin and other conditions that involve inhibiting the synthesis and/or release of renin from mast cells and/or inhibiting the activity of renin after release from mast cells. The methods of the invention can also involve inhibiting elements of the local renin-angiotensin system (e.g. inhibiting ACE and angiotensin II receptors), thereby inhibiting angiotensin II produced locally from mast-cell-derived renin

### RELATED APPLICATION

This application claims priority to U.S. Provisional Application Ser. No. 60/512,142, filed Oct. 17, 2003, the disclosure of which is incorporated herein in its entirety.

### GOVERNMENT FUNDING

The invention described herein was developed with the support of the National Institutes of Health, under Contract Nos. 1R01 HL34215 and 1R01 DK060726. The United States Government has certain rights in the invention.

### FIELD OF THE INVENTION

The invention relates to a discovery that mast cells are a unique source of extra-renal renin. The invention provides methods for controlling mast cell-derived renin, activity of mast-cell derived renin, release of mast cell-derived renin and the formation of angiotensin II by mast cell-derived renin. Such methods can be used to treat a variety of conditions including those associated with mast cell hyperplasia. For example, the methods of the invention can be used to treat cardiac ischemia, asthma, irritable bowel syndrome, organ fibrosis (liver, lung, kidney, heart and skin), inflammatory diseases, and any pathology associated with increased numbers of mast cells and the coincident release of renin and/or the local formation of angiotensin II.

### BACKGROUND OF THE INVENTION

Traditionally, the renin-angiotensin system (RAS) has been perceived as occurring largely through factors brought together by the circulatory system from disparate organ systems. Hence, liver-derived angiotensinogen was believed to be cleaved only by renin formed in the kidney to generate angiotensin I (ANG I) that was then converted to the biologically active product angiotensin II (ANG II) by angiotensin-converting enzyme (ACE). Such cleavage and conversion steps were believed to occur within the general circulation and at the luminal surface of the vascular endothelium, respectively. Circulating angiotensin II is known to regulate blood pressure, plasma volume, sympathetic neural activity, and thirst responses. Campbell, D. J. Circulating and tissue angiotensin systems. J Clin Invest 79: 1-6, 1987; Peach, M. J. Renin-angiotensin system: biochemistry and mechanisms of action. Physiol Rev. 57: 313-370, 1977.

In recent years, workers have reported that tissues such as the heart are capable of producing angiotensin II. Some research indicates that angiotensin I can be synthesized in situ within the heart and that some cardiac angiotensin II is produced by conversion of locally produced, rather than blood-derived, angiotensin I. Yet, no cellular source for locally produced renin has been identified. And little or no evidence exists that biologically significant amounts of renin are produced outside of the kidneys. Nor do methods exist for controlling the renin-angiotensin system within localized tissues so that diseases peculiar to those tissues and associated with renin activity can be treated.

Hence, a need exists for greater understanding of the renin-angiotensin system and for methods of controlling that system in discrete tissues.

### SUMMARY OF THE INVENTION

The invention relates to the discovery that renin is present in mast cells and can act in a localized manner to initiate and/or exacerbate a number of conditions related to the localized formation of angiotensin II. Thus, the invention provides methods for regulating production and release of renin by mast cells. In other embodiments, the invention provides methods for inhibiting the activity of mast cell produced renin. In still other embodiments, the invention provides methods for inhibiting the formation of angiotensin by mast cell derived renin and/or blocking angiotensin receptor activation and activity. Such methods are useful for treating and preventing a variety of cardiac, vascular, lung, bladder, skin, liver, kidney, pancreas, gastrointestinal and other conditions.

The methods of the invention involve inhibiting the production and release of renin from mast cells, inhibiting the activity of renin after release from mast cells and/or inhibiting the formation of angiotensin from mast cell derived renin. Such inhibition can be achieved locally by localized administration of any available mast cell stabilizer or renin inhibitor. Moreover, such mast cell stabilizers and renin inhibitors can be combined in a composition for localized administration to selected tissues with a variety other active agents including angiotensin-converting enzyme (ACE) inhibitors or agents that block Na⁺/H⁺ exchanger (NHE) or angiotensin receptor (e.g., AT₁R) activity.

Another aspect of the invention is an siRNA comprising any one of SEQ ID NO:58-61, wherein the siRNA can inhibit renin RNA function

Another aspect of the invention is a composition comprising a carrier and an siRNA that can inhibit renin RNA function, wherein the composition is formulated for localized delivery. Such an siRNA can be complementary to a nucleic acid sequence comprising SEQ ID NO:53 or 54. In another embodiment, the siRNA can be complementary to any one of SEQ ID NO:55-57. Examples of actual siRNA molecules that can inhibit the function of renin mRNA are nucleic acids comprising any one of SEQ ID NO:58-61.

Another aspect of the invention is a composition comprising a carrier and an inhibitor of renin, wherein the composition is formulated for localized delivery to a tissue. Any available inhibitor of renin can be used in these compositions.

Another aspect of the invention is a composition comprising a carrier and an inhibitor of mast cell degranulation, wherein the composition is formulated for inhibiting renin release from mast cells at localized sites in a tissue. Any available mast cell degranulation inhibitor can be used in these compositions.

The compositions of the invention are preferably formulated for local administration to heart, vascular, lung, bladder, skin, liver, kidney, pancreas, or gastrointestinal tissues.

### BRIEF DESCRIPTION OF THE DRAWINGS

FIG. 1 schematically illustrates a central discovery of the invention that mast cells are a unique extra-renal source of renin. Because renin is the rate-limiting factor in the production of angiotensin, the discovery of renin in mast cells has dramatic consequences, especially those relating to the localized production of angiotensin in numerous tissues. As depicted in this scheme, mast cell degranulation is the pivotal event in the local formation of angiotensin. The renin that is released from mast cells cleaves the decapeptide angiotensinogen (Aogen) to form the octapeptide angiotensin I (ANG I), which then gets converted to the more active angiotensin II (ANG II) by angiotensin-converting enzyme (ACE). The newly formed ANG II is then free to bind to angiotensin receptors found on numerous cell types, including mast cells, nerves, smooth muscle, and the like. ANG II formed locally from mast-cell derived renin may then act in a multitude of ways and can lead, for example, to inflammation, smooth muscle constriction, fibrosis, etc. thereby significantly contributing to pathological states.

FIG. 2A illustrates that an anti-renin antibody recognizes renin protein at its classical source—the vascular pole of the glomerulus of the kidney. The polyclonal anti-renin antibody was diluted 1:500 and exhibited specific binding to rat kidney at the vascular pole of the glomerulus (FIG. 2A, arrow). Bar=10 μm. This is a positive control showing that this anti-renin antibody recognizes the renin producing cells in kidney.

FIG. 2B illustrates the specificity of this poly-clonal anti-renin antibody—no staining was seen in kidney sections exposed to the polyclonal anti-renin Ab (1:500) that were pre-adsorbed with an excess of human renin. Bar=10 μm.

FIG. 2C and 2D show a sub-population of cells with a granular appearance in sections of rat ventricle that were reacted with the polyclonal anti-renin Ab (1:500). FIG. 2C provides a view using a 40× objective. FIG. 2D provides a view with a 100× objective. Bars=10 μm (FIG. 2C) and 4 μm (FIG. 2D). These immuno-positive cells were visually identified as mast cells. No other cell type (i.e. myocytes, nerves) in the heart sections were stained with the anti-renin antibody.

FIG. 2E demonstrates that mast cells exist in heart. A classical histochemical stain for mast cells was used for mast cell identification, toluidine blue. As shown, cells within rat ventricle heart sections stain with toluidine blue (Bar=20 μm).

FIG. 3 further illustrates that the cell type immuno-positive for anti-renin antibody in the heart sections, is a mast cell.

FIG. 3A and 3A¹ demonstrates that the cells that are renin-positive also stain for toluidine blue, the classic histochemical stain for mast cells (FIG. 3A and A¹). A representative section of rat ventricle was stained with both the polyclonal anti-renin Ab (FIG. 3A) and toluidine blue (FIG. 3A¹). Bar=5 μm.

FIG. 3B and 3B¹ illustrates that pre-absorption of the anti-renin antibody essentially eliminates binding of this antibody to a section of rat ventricle, thereby illustrating that such binding is renin-specific. FIG. 3B-B¹ shows a section of rat ventricle reacted with the polyclonal anti-renin antibody (1:500) that had been pre-adsorbed with an excess of human renin (B) and stained with toluidine blue (B¹). The toluidine-blue-stained mast cells (FIG. 3B¹) did not immunoreact with the pre-adsorbed anti-renin antibody (see asterisks in FIG. 3B).

FIG. 3C shows that a cell immuno-positive for histamine, a known component of mast cells, is also stained with the anti-renin antibody. A cardiac mast cell co-labeled with the monoclonal anti-renin antibody (red, closed arrowheaad) (1:100) and an anti-histamine antibody (green, open arrowhead) (1:500). Bar=10 μm. Areas stained with both antibodies appear yellow.

FIG. 3D illustrates by Western blot, that the anti-renin antibody does not cross react with the protease, cathepsin D (CD), which is also found in mast cells. This figure also shows that cathepsin D antibody does not cross react with renin. A Western blot of rat kidney homogenate (Kid) (20 μg/lane) and pure cathepsin D (CD) (500 μg/lane) was probed with the polyclonal anti-renin antibody (1:12,500) and an anti-cathepsin D (CD) antibody (1:100).

FIG. 3E shows a mast cell co-stained with an anti-renin antibody and an anti-cathepsin D antibody. The cardiac mast cell is co-labeled with anti-cathepsin D antibody (green, open arrowhead) (1:500) and the polyclonal anti-renin antibody (red, closed arrowhead) (1:500). Bar=5 μm.

FIG. 4 shows that mast cells synthesize renin that is homologous to kidney renin, and that mast cell renin is active, i.e. able to cleave angiotensinogen to angiotensin I, when released from mast cells. To demonstrate this, the chymase-deficient human mast-cell line, HMC-1 (Nilsson et al., Scand. J. Immunol. 39: 489-498 (1994)) was utilized.

FIG. 4A illustrates that HMC-1 mast cells express renin. Total RNA extracted from human kidney (left) and the human mast cell line HMC-1 (right) was reverse transcribed. cDNA was amplified by PCR using specific primers for human renin (lanes 1 and 4) and β-actin controls (lanes 2 and 5). A 100 base pair marker ladder (m) was run in the lane between the kidney and HMC-1 RT-PCR products.

FIG. 4B provides the sequence of the cDNA (SEQ ID NO:66) product generated as described in FIG. 4A. The DNA band corresponding to the HMC-1 RT-PCR product was excised from the gel, the DNA was isolated and then subjected to DNA sequencing at the Rockefeller University Sequencing Facility. The reported nucleotide sequence was then compared to the known sequence of Homo sapiens renin mRNA by BLAST analysis. As shown in FIG. 4B, there was 100% homology between the sequence from HMC-1 and human renin, establishing a precedent for the presence of renin in mast cells.

FIG. 4C provides a Western blot of rat kidney homogenate (20 μg/lane) and HMC-1 cells (50 μg/lane) probed for renin with the polyclonal anti-renin Ab (1:12,500). The arrow shows an approximate 42 kD band corresponding to renin.

FIG. 4D illustrates immunocytochemical staining of HMC-1 cells with the polyclonal anti-renin antibody (1:400). Bar=10 μm.

FIG. 4E demonstrates that ANG I was formed (renin activity, pg/ml/hr), from an HMC-1 cell releasate that had been incubated with increasing concentrations of human angiotensinogen (Aogen), with or without the selective renin inhibitor BILA2157 (100 nM), (mean±SEM; n=3).

FIG. 4F further illustrates that ANG I formation is inhibited when the BILA2157 inhibitor is present in HMC-1 cell releasate. Renin activity was measured as pg ANG I/ml/hr/10⁵ cells). Releasate was incubated with 240 nM Aogen in the absence or presence of BILA2157 (100 nM), (mean±SEM; n=12).

FIG. 5A-D illustrates inhibition of renin synthesis in the HMC-1 cells using a gene-specific small interfering RNA (siRNA).

FIG. 5A, C and D show that a renin-specific siRNA inhibits ANG I formation in HMC-1 cells. Renin activity (pg/ ANG I formed/ml/hr/10⁵ cells) was measured on HMC-1 cell releasates from a defined population of HMC-1 cells before and 48 hours after transfection either with siRNA designed for degrading sequence-specific renin MRNA (FIG. 5A), transfection medium (FIG. 5C, gene silencer), or a scrambled siRNA (FIG. 5D, the scrambled siRNA had no specificity for renin nucleic acids).

FIG. SB shows that in HMC-1 cells transfected with siRNA specific for renin, the renin immunostaining with polyclonal anti-renin antibody (1:400), was considerably diminished compared to HMC-1 cells not exposed to the siRNA.

FIG. 6 schematically illustrates the paracrine pathway believed to be responsible for local ANG II production in the heart, which in turn leads to release of excessive norepinephrine (NE) from cardiac sympathetic nerve endings. According to the invention, degranulation of mast cells, as occurs in myocardial ischemia, releases active renin protein into the cardiac interstitium, where it may bind to the extracellular surface of cell membranes. This bound renin then interacts with the angiotensinogen (Aogen) present in the interstitial fluid, cleaving it to angiotensin I (ANG I). The conversion of ANG I to ANG II is mediated by the available ACE, also reported to exist in the interstitial fluid (de Lannoy et al., J. Hypertens. 19: 959-965, 2001). The resultant ANG II may then interact with angiotensin receptor. As illustrated in this schematic diagram, activation of the ANG II receptor subtype, AT₁R, on cardiac sympathetic nerve endings, leads to the stimulation of the Na⁺/H⁺ exchanger (NHE). Stimulation of NHE elevates cytosolic Na⁺ levels in the nerve ending, causing excessive release of norepinephrine via the norepinephrine transporter (NET; i.e., “carrier-mediated NE release”). Sympathetic over-activity, accompanied by excessive NE release is a recognized cause of cardiac dysfunction in myocardial ischemia, congestive heart failure, and hypertension. Myocardial infarction is often accompanied by arrhythmias with high morbidity and mortality. Thus, according to the invention, mast cell degranulation in myocardial ischemia is a pivotal event in the activation of a cardiac renin angiotensin system (RAS) and leads to excessive release of norepinephrine, which may cause problematic or even lethal cardiac arrhythmias and other cardiac conditions.

FIG. 7 shows that renin-containing mast cells are found in close proximity to nerves. A confocal image of rat ventricle (0.5 μm) co-labeled with the polyclonal anti-renin Ab (red) (1:500) and an anti-synapsin Ab (green) (1:300). The mast cells stained red and cardiac nerves stained green. Bar=40 μm.

FIG. 8A-C illustrates that norepinephrine is released in a dose-dependent manner from guinea pig cardiac sympathetic nerve endings incubated with increasing amounts of human angiotensinogen (2.4 - 240 nM). FIG. 8A provides the norepinephrine release of hearts prior to isolation of the cardiac sympathetic nerve endings. Hearts were either untreated (control), or challenged with the mast cell degranulating compound, 48/80 (300 μg bolus injection), in the presence or absence of the mast cell stabilizing agent, lodoxamide (10 μM). FIG. 8B-C provides the results for isolated cardiac sympathetic nerve endings. The cardiac sympathetic nerve endings were incubated with the selective renin inhibitor BILA2157 (FIG. 8B, 10 nM) or the ACE inhibitor, enalaprilat (FIG. 8C, 30 nM), or an ANG II AT₁ receptor antagonist, EXP 3174 (FIG. 8C, 10 nM). Basal release of norepinephrine was 1.45 ±0.03 pmol/mg protein.

FIG. 9 illustrates that mast cell degranulation with 48/80 in normoxic guinea pig hearts perfused ex vivo in a Langendorff apparatus causes the release of active renin into the coronary effluent. Exposure to 48/80 and the renin inhibitor BILA 2157 (100 nM) markedly diminished the amount of renin in the coronary effluent as did the presence of the mast cell stabilizer, lodoxamide (10 μM),

FIG. 10A shows that a positive chronotropic response occurs upon administration of the mast cell degranulation agent 48/80 to isolated normoxic guinea pig hearts. The presence of BILA 2157 (100 nM), a renin inhibitor, or lodoxamide (30 μM), a mast cell stabilizing agent diminishes the chronotropic response. Inset: Areas under each chronotropic curve. FIG. 10B illustrates that coronary norepinephrine (NE) overflow is observed in control hearts in response to mast cell degranulation with 48/80.

FIG. 11 illustrates that ischemia-reperfuision causes the release of active renin, as detected by ANG I formation, into the coronary effluent of guinea pig hearts perfused ex vivo in a Langendorff apparatus. Mast cell stabilization with lodoxamide or renin inhibition with BILA 2157 prevented renin release. Hearts were subjected to 20 minutes global ischemia followed by 45 minutes reperfusion in the absence (n=7) or in the presence of lodoxamide (10 μM; n=5), or BILA 2157 (100 nM); n=3). Renin activity, as detected by ANG I formation, was measured by RIA. Bars are means±SEM.

FIG. 12 shows that the renin inhibitor BILA 2157 and the mast cell stabilizer lodoxamide prevent ventricular fibrillation (VF) in an ex vivo ischemia-reperfusion model in the guinea pig heart.

FIG. 13A graphically illustrates that the duration of ventricular fibrillation (VF) following ischemia is positively correlated with the amount of renin overflow into the coronary effluent (n=8 hearts) in the ischemia-reperfusion model.

FIG. 13B shows that essentially no ventricular fibrillation is observed in hearts following ischemia when the hearts were perfused with the renin inhibitor BILA 2157 (100 nM) (n=4).

FIG. 14 shows that the duration of ventricular fibrillation occurring during reperfusion of 8 isolated guinea pig hearts following ischemia is positively correlated with the amount of norepinephrine (NE) released into the coronary effluent.

FIG. 15A-D show that heart sections from WBB6F₁-W/W^{V} knock out mice, which are known to be about 90% mast cell deficient, exhibit essentially no detectable anti-renin antibody staining. Sections of mouse hearts from WBB6F₁,-W/W^{V} knock out (KO) and WBB6F1-⁺/⁺ congenic control (CC) animals were stained with the anti-renin antibody and viewed with 40× (FIG. 15A and B) and 100× (FIG. 15C and D) objectives. Arrows show renin-positive mast cells found in CC sections only. No mast cell staining was observed in sections studied from KO hearts. Bars=10 μM (FIG. 15A and B) and 4 μM (FIG. 15C and D).

FIG. 16 illustrates that both renin overflow and ventricular fibrillation (VF) are substantially absent in KO mouse hearts subjected to ischemia-reperfuision ex vivo in a Langendorff apparatus. Abscissa: Renin overflow into the coronary effluent during reperfusion following 30 minute stop-flow global ischemia. Ordinate: Duration of VF occurring during reperfusion. Data plotted are the mean values for two KO and CC hearts observed.

FIG. 17 provides representative ECG traces showing the lack of ventricular fibrillation in KO mouse hearts subjected to ischemia-reperfusion ex vivo. In contrast, control (CC) hearts exhibited pronounced ventricular fibrillation.

FIG. 18A-B provides representative traces illustrating the Na⁺-dependent recovery of intracellular pH after an acute acid pulse in synaptosomes from guinea pig heart either in the absence (FIG. 18A) or presence (FIG. 19B) of angiotensin II (10 nM). The dashed lines represent the slopes of the Na⁺-dependent increase in pH.

FIG. 18C provides a bar graph illustrating that the mean Na⁺/H⁺ exchanger activities in synaptosomes increase when the synaptosomes are treated with norepinephrine (NE, 1 μM) or angiotensin II (10 nM).

FIG. 19 provides a bar graph illustrating that the mean activity of Na⁺/H⁺ exchanger in human neuroblastoma SH-SY5Y-AT_{1A} cells increases when the cells are treated with angiotensin II (10 nM) but that exposure of the cells to BAPTA-AM (10 μM) prevented the angiotensin II-induced increase in the Na⁺/H⁺ exchanger activity.

FIG. 20 provides a bar graph illustrating that norepinephrine release from guinea pig synaptosomes incubated with angiotensin II (100 nM) is blocked by the PLC inhibitor U-73122 (1 μM), the inhibitor of the Na⁺/H⁺ exchanger 5-(N-ethyl-N-isopropyl)-amiloride (EIPA) (30 μM) and the norepinephrine transporter inhibitor desipramine (DMI) (300 μM). Bars (mean±SEM; n=6) represent the percent increase in norepinephrine release.

FIG. 21A-B provides representative traces illustrating the intracellular Ca²⁺ as a function of time after a pulse of angiotensin II in human neuroblastoma SH-SY5Y-AT_{1A} cells either in the absence (A) or presence (B) of the PLC inhibitor U-73122 (1 μM).

FIG. 22 provides a bar graph illustrating the angiotensin II-evoked carrier-mediated release of [³H]N-methyl-4-phenylpyridinium (MPP⁺) from human neuroblastoma SH-SY5Y-AT_{1A} cells. The angiotensin type 1 receptor inhibitor, EXP3174, blocks this angiotensin II-induced MPP⁺ release. The protein kinase C activator PMA increases angiotensin II induced MPP+release, which also is blocked by EXP3174.

FIG. 23A-B shows that renin-positive mast cells in rat bronchus (FIG. 18A, viewed with 40× objective) exist in close proximity to AT₁R-positive smooth muscle cells in rat bronchial tissues (FIG. 18B, viewed with 100× objective).

FIG. 24 shows that mast cell degranulation with 48/80 leads to contraction of rat bronchial smooth muscle. The AT₁R antagonist, EXP 3174, inhibited the smooth muscle contraction, illustrating that ANG II derived from mast-cell renin is responsible for the bronchoconstriction (n=3). In contrast, the smooth muscle contraction induced by depolarization with K⁺ (see inset) was not affected by EXP 3174 (data not shown).

FIG. 25 illustrates that renin-positive mast cells (arrows) exist in a variety of tissues. Examples include lung, stomach, ileum, liver. All sections were viewed with a 40× objective.

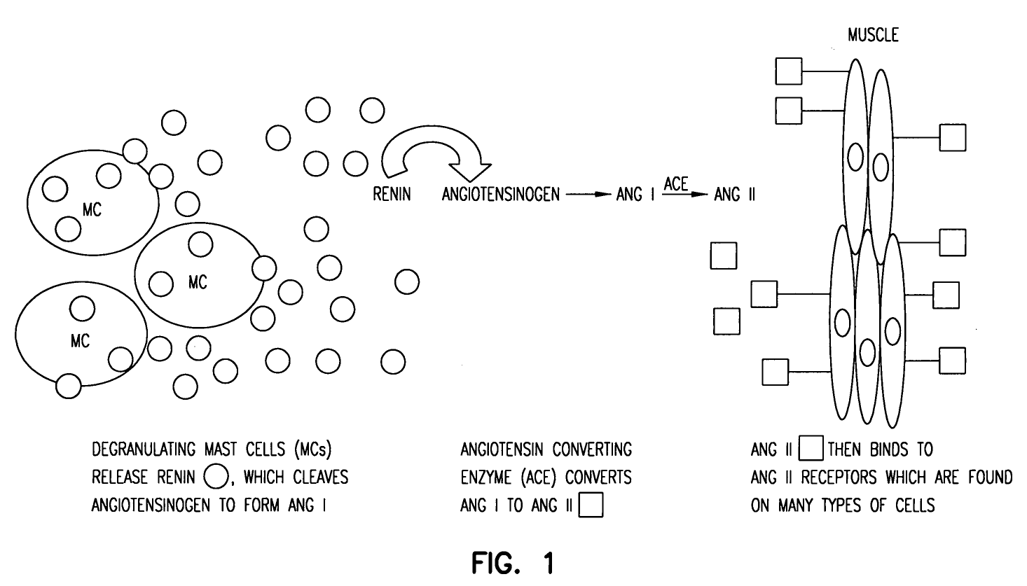

### DETAILED DESCRIPTION OF THE INVENTION

According to the invention, mast cells located throughout the body and in essentially all major organs can produce renin. Moreover, mast cells can respond to local signals and produce renin in a localized manner in response to those localized signals. The invention provides methods of controlling mast cell generated renin, for example, to treat a variety of conditions including those associated with mast cell hyperplasia. Examples of conditions that can be treated by the methods of the invention include chronic obstructive pulmonary disease, Corpulmonale, bronchiectasis, acute respiratory distress syndrome, bronchiolitis obliterans-organizing pneumonia, cystic fibrosis, interstitial lung diseases, silicosis, sarcoidosis, lung cancer, tuberculosis, gastritis, peptic ulcer, hepatocellular carcinoma, ulcerative colitis, Crohn's disease, liver cirrhosis, hepatitis, pancreatitis, atherosclerosis, myocardial infarction, congenital heart disease, myocarditis, cardiomyopathy, diabetes, thyroiditis, osteoporosis, glomerulonephritis, nephropathy, multiple sclerosis, rheumatoid arthritis, osteoarthritis, rheumatic arthritis, congestive heart failure, cardiac hypertrophy, hypertension, cardiomyopathy, asthma, , endometriosis, brain infarction, organ fibrosis (liver, lung, kidney, heart and skin), interstitial cystitis, pancreatic cancer, cardiomyopathy, and any pathology associated with increase numbers of mast cells that is coincident with the release of renin and that leads to angiotensin formation, and any disease associated with pro-inflammatory, fibrotic, constrictive, and/or any adverse effects of locally produced angiotensin.

Definitions

As used herein, asthma is a condition characterized by episodes of airway obstruction due to bronchial smooth muscle constriction, bronchial wall edema, and mucous plugging. Pathological hallmarks of asthma include bronchocentric inflammation and hyperplasia of the structural elements of the airway including smooth muscle and vasculature.

As used herein “congestive heart failure” refers to a syndrome characterized by left ventricular dysfunction, reduced exercise tolerance, impaired quality of life, and markedly shortened life expectancy. Decreased contractility of the left ventricle leads to reduced cardiac output with consequent systemic arterial and venous vasoconstriction. This vasoconstriction, which appears to be mediated, in part, by the renin-angiotensin system, promotes the vicious cycle of further reductions of stroke volume followed by an increased elevation of vascular resistance. In addition, locally produced angiotensin leads to re-modeling of the myocardium leading to cardiac hypertrophy that further exacerbates congestive heart failure.

As used herein, the term “mammal” refers to any animal classified as a mammal, including humans, domestic and farm animals, and zoo, sports, or pet animals such as dogs, horses, cats, cows, etc. Preferably, the mammal herein is human.

The term myocardial infarction is used to describe necrosis or death of myocardial cells. Atherosclerotic heart disease is the most common underlying cause of myocardial infarction.

As used herein, myocardial ischemia is a condition in which oxygen deprivation to the heart muscle is accompanied by inadequate removal of metabolites because of reduced blood flow or perfusion. Atherosclerosis of the larger coronary arteries is the most common anatomic condition to diminish coronary blood flow.

Renin

According to the invention, mast cells synthesize, contain and secrete renin and/or renin-like enzymes.

Several examples of nucleotide and amino acid sequences for renin are available, for example, in the database provided by the National Center for Biotechnology Information (NCBI) (see website at www.ncbi.nlm.nih.gov). One example of a sequence for renin is the amino acid sequence at NCBI accession number AAA60363 (gi: 190994). See website at ncbi.nlm.nih.gov. The amino acid sequence for this renin protein is provided below (SEQ ID NO:1).

| | |
|---|---|
| 1 | MDGWRRMPRW GLLLLLWGSC TFGLPTDTTT FKRIFLKRMP |
| 41 | SIRESLKERG VDMARLGPEW SQPMKRLTLG NTTSSVILTN |
| 81 | YMDTQYYGEI GIGTPPQTFK VVFDTGSSNV WVPSSKCSRL |
| 121 | YTACVYHKLF DASDSSSYKH NGTELTLRYS TGTVSGFLSQ |
| 161 | DIITVGGITV TQMFGEVTEM PALPFMLAEF DGVVGMGFIE |
| 201 | QAIGRVTPIF DNIISQGVLK EDVFSFYYNR DSENSQSLGG |
| 241 | QIVLGGSDPQ HYEGNFHYIN LIKTGVWQIQ MKGVSVGSST |
| 281 | LLCEDGCLAL VDTGASYISG STSSIEKLME ALGAKKRLFD |
| 321 | YVVKCNEGPT LPDISFHLGG KEYTLTSADY VFQESYSSKK |
| 361 | LCTLAIHAMD IPPPTGPTWA LGATFIRKFY TEFDRRNNRI |
| 401 | GFALAR |

Another example of an amino acid sequence for a human renin protein is available in the NCBI database at accession number AAD03461 (gi:337340); the nucleotide sequence for this human renin protein can be found at accession number AH007216 (gi:337339). See website at ncbi.nlm.nih.gov. The amino acid sequence for this renin protein is provided below (SEQ ID NO:2).

| | |
|---|---|
| 1 | MDGWRRMPRW GLLLLLWGSC TFGLPTDTTT FKRIFLKRMP |
| 41 | SIRESLKERG VDMARLGPEW SQPMKRLTLG NTTSSVILTN |
| 81 | YMDTQYYGEI GIGTPPQTFK VVFDTGSSNV WVPSSKCSRL |
| 121 | YTACVYHKLF DASDSSSYKH NGTELTLRYS TGTVSGFLSQ |
| 161 | DIITVGGITV TQMFGEVTEM PALPFMLAEF DGVVGMGFIE |
| 201 | QAIGRVTPIF DNIISQGVLK EDVFSFYYNR DSENSQSLGG |
| 241 | QIVLGGSDPQ HYEGNFHYIN LIKTGVWQIQ MKGVSVGSST |
| 281 | LLCEDGCLAL VDTGASYISG STSSIEKLME ALGAKKRLFD |
| 321 | YVVKCNEGPT LPDISFHLGG KEYTLTSADY VFQESYSSKK |
| 361 | LCTLAIHAMD IPPPTGPTWA LGATFIRKFY TEFDRRNNRI |
| 401 | GFALAR |

Many more sequences for renin are available, for example, at the ncbi.nlm.nih.gov website.
Renin Inhibitors

Renin inhibitors that can be used in the invention include any renin inhibitor available to one of skill in the art. Renin inhibitors that can be used in the invention include, for example, aliskiren, remikiren, ankiren and enalkiren. Aliskiren, is a recently described, novel and orally-effective renin inhibitor. See Wood et al., Biochem. Biophys. Res. Comm., 308(4):698-705. Aliskiren, is available from Speedel Pharma in Basel, Switzerland. Other examples of renin inhibitors include those having the formula I (SEQ ID NO:3):

| | |
|---|---|
| Xaa₁-Xaa₂-Xaa₃-Xaa₄-Xaa₅-Y | I |

wherein:

each Xaa₁, Xaa₂, Xaa₃, Xaa4 and Xaa₅ is separately an individual amino acid residue

each Xaa₁, Xaa₂, and Xaa4 may be the same or different and are selected from the group consisting of phenylalanine (Phe), 4-chlorophenylalanine (Phe(4-C1)), 4-fluorophenylalanine (Phe(4-F)), 4-bromophenylalanine (Phe(4-Br)), methoxyphenylalanine (Phe(OMe)), tyrosine (Tyr), 4-iodo-phenylalanine (Phe(4-I)) and ortho-methyl tyrosine (Tyr(ortho-Me));

Xaa₃ is valine (Val), threo-α-3-chlorobutyric acid (Bca) or threonine (Thr)

Xaa₅ is lysine (Lys) or arginine (Arg);

Y is NH2 (in which case the pentapeptide is in the form of the carboxy-amide), NHR, wherein R is C₁-C₄ alkyl (in which case the pentapeptide is in the form of the ester), OH (in which case the pentapeptide is in the form of the free acid), OR,₁, wherein R₁ is C₁-C₄ alkyl, or O-Z, wherein Z is a cation (in which case the pentapeptide is in the form of the C-terminal salt). Useful cations are alkaline or alkaline earth metallic cations (e.g. Na, K, Li, Ca, etc.) or amine cations (e.g. tetralkyl ammonium, trialkyl ammonium, where alkyl can be C₁-C₁₂.

The pentapeptides may be in the form of the free amines (on the N-terminus) or acid addition salts thereof. Common acid addition salts are hydrohalic acid salts, for example HBr, HF, or, preferably, HCl.

In some embodiments the pentapeptides preferably have a C-terminal amide. In other embodiments, the pentapeptides have a Lys as the C-terminal amino acid (Xaa₅).

Representative pentapeptides that can be used in the present invention are:

| | |
|---|---|
| H₂N-Phe-Phe-Val-Tyr-Lys-CONH₂ | (SEQ ID NO:4) |
| H₂N-Tyr-Phe-Val-Tyr-Lys-CONH₂ | (SEQ ID NO:5) |
| H₂N-Phe(4Cl)-Phe-Val-Tyr-Lys-CONH₂ | (SEQ ID NO:6) |
| H₂N-Phe-Tyr-Val-Tyr-Lys-CONH₂ | (SEQ ID NO:7) |
| H₂N-Phe-Phe(4Cl)-Val-Tyr-Lys-CONH₂ | (SEQ ID NO:8) |
| H₂N-Phe-Tyr(Me)-Val-Tyr-Lys-CONH₂ | (SEQ ID NO:9) |
| H₂N-Phe-Phe(4-I)-Val-Tyr-Lys-CONH₂ | (SEQ ID NO:10) |
| H₂N-Phe-Phe-Thr-Tyr-Lys-CONH₂ | (SEQ ID NO:11) |
| H₂N-Phe-Phe-Bca-Tyr-Lys-CONH₂ | (SEQ ID NO:12) |
| H₂N-Phe-Phe-Val-Phe-Lys-CONH₂ | (SEQ ID NO:13) |
| H₂N-Phe-Phe-Val-Phe(4Cl)-Lys-CONH₂ | (SEQ ID NO:14) |

In some embodiments, the inhibitor employed is H₂N-Phe-Phe(4Cl)-Val-Tyr-Lys-CONH₂ (SEQ ID NO:8), and its acid addition salts.

Other examples of renin inhibitors are provided in the following table.

| | |
|---|---|
| Renin Inhibitor | Reference |

| | |
|---|---|
| Iva His Pro Phe His Sta Leu Phe | Hypertension 6:I-111, 1984 |
| SEQ ID NO:15 | |
| Pepstatin | Science 175: 656, 1972 |
| 2-[4-(4′-chlorophenoxy)- | J Pharm Exp. Ther. 203: |
| phenoxyacetyl-amino] ethyl- | 485, 1977 |
| phosphoryl ethanolamine (PE 104) | |
| CH₂CH (CH₃)₂ | Hypertension 4 (Suppl II: |
| His Pro Phe His CHCH₂ Leu Val Tyr | II-59, 1982 |
| SEQ ID NO:67 | |
| Cyclic Cys His Pro Phe His Cys | Biochem. J. 205: 43, 1982 |
| Leu Val Tyr Ser | |
| SEQ ID NO:16 | |
| Cyclic Lys Cys His Pro Phe His | ″ |
| Cys Leu Val Tyr Ser | |
| SEQ ID NO:17 | |
| Cyclic Cys His Pro Phe His Cys | ″ |
| Leu Val Tyr Lys | |
| SEQ ID NO:18 | |
| Cyclic Lys Cys His Pro Phe His | Biochem. J. 205: 43, 1982 |
| Val Ile His Ser | |
| SEQ ID NO:19 | |
| Pro His Pro Phe His Phe Phe | Biochem. Biophys. Res. |
| Val Tyr Lys | Commun. 97(1): 230, 1980 |
| SEQ ID NO:20 | |
| BzHis Pro Phe His Leucinal | Biochem. Biophys. Res. |
| (Benzyloxycarbonyl equals Bz) | Commun. 118(3): 929, 1984 |
| SEQ ID NO:21 | |
| BzPro Phe His Leucinal | Biochem. Biophys. Res. |
| SEQ ID NO:22 | Commun. 118(3): 929, 1984 |
| BzPhe His Leucinal | Biochem. Biophys. Res. |
| | Commun. 118(3): 929, 1984 |
| BzHis Leucinal | Biochem. Biophys. Res. |
| | Commun. 118(3): 929, 1984 |
| Bz[3-(1′-naphthyl)Ala]His | Biochem. Biophys. Res. |
| Leucinal | Commun. 118(3): 929, 1984 |
| His Pro Phe His D-Leu Leu Val Tyr | Biochemistry 12: 3877, 1973 |
| SEQ ID NO:23 | |
| Asp Arg Val Tyr Ile His Pro Phe | Biochem. Biophys. Res. |
| His Leu Leu Val Tyr Ser | Commun. 54: 1365, 1973 |
| SEQ ID NO:24 | |
| Pro His Pro Phe His Phe Phe Val | Biochemistry 14: 3892, |
| Tyr | 1975 |
| SEQ ID NO:25 | |
| D-His Pro Phe His Leu^{R}* | Hypertension 4 (Suppl II): |
| Leu Val Tyr | II-59, 1982 |
| SEQ ID NO:26 | |
| BocHis Pro Phe His Sta Leu | Nature 303: 81, 1983 |
| Phe | |
| SEQ ID NO:27 | |
| Pro His Pro Phe His Leu^{R}* | Nature 299: 555, 1982 |
| Val Ile His Lys | |
| SEQ ID NO:28 | |
| Iva His Pro Phe His Sta Ile | Nature 303: 81, 1983 |
| Phe | |
| SEQ ID NO:29 | |
| Boc Phe His Sta Ala Sta | Peptides: Structure and |
| SEQ ID NO:30 | Function, ed V.J. Hruby, |
| | Rockford, Illinois, Pierce |
| | Chemical Co., 1984 |
| Arg Ile Pro-OMe | Proc. Natl. Acad. Sci. USA |
| | 81: 48, 1984 |
| BocLeu Lys Lys Met ProOMe | Proc. Natl. Acad. Sci. USA |
| SEQ ID NO:31 | 81: 48, 1984 |
| BocArg Ile Pro Leu Lys Lys | Proc. Natl. Acad. Sci. USA |
| Met ProOMe | 81: 48, 1984 |
| SEQ ID NO:32 | |
| BocGlu Arg Ile Pro Leu Lys | Proc. Natl. Acad. Sci. USA |
| Lys Met ProOMe | 81: 48, 1984 |
| SEQ ID NO:33 | |
| Ac Val Val Sta Ala Sta | Proc. Natl. Acad. Sci. USA |
| SEQ ID NO:34 | 81: 48, 1984 |
| Iva Val Val Sta Ala Sta Glu | Proc. Natl. Acad. Sci. USA |
| SEQ ID NO:35 | 81: 48, 1984 |
| L-α-hydroxy-isocaproyl-Leu Val | Biochem. Biophys. Res. |
| PheOMe | Commun. 100: (1) 177, 1981 |
| L-α-hydroxyisovaleryl Leu PheOMe | Biochem. Biophys. Res. |
| | Commun. 100: (1) 177, 1981 |
| L-α-hydroxyisovaleryl Leu Val | Biochem. Biophys. Res. |
| PheOMe | Commun. 100: (1) 177, 1981 |
| L-α-hydroxyisovaleryl Leu Val | Biochem. Biophys. Res. |
| Phe(NO₂)OMe | Commun. 100: (1) 177, 1981 |
| L-α-hydroxy-isovaleryl Leu Val | Biochem. Biophys. Res. |
| Tyr(Me)OMe | Commun. 100: (1) 177, 1981 |
| L-α-hydroxy-isovaleryl Leu Val | Biochem. Biophys. Res. |
| Phe(NH₂)OMe | Commun. 100: (1) 177, 1981 |
| Lac Leu Val Phe(NO₂)OMe | Biochem. Biophys. Res. |
| | Commun. 100: (1) 177, 1981 |
| Lac Leu Val Tyr(Me)OMe | Biochem. Biophys. Res. |
| | Commun. 100: (1) 177, 1981 |
| Lac Leu Val Phe(NH₂)OMe | Biochem. Biophys. Res. |
| | Commun. 100: (1) 177, 1981 |
| HLeu Val Phe(NO₂)OMe | Biochem. Biophys. Res. |
| | Commun. 100: (1) 177, 1981 |
| HLeu Val Tyr(Me)OMe | Biochem. Biophys. Res. |
| | Commun. 100: (1) 177, 1981 |
| Heparin | J. Clin. Endocrinol. 27: |
| | 699, 1967 |
| Deoxycholic acid | Biochem. Pharmacol. 20: |
| | 914, 1971 |
| Phosphotidyl ethanolamine | Proc. Soc. Exp. Biol. Med. |
| where acid substituents | 155: 468, 1977 |
| thereof are each | |
| independently arachidonic | |
| acid, linolenic acid or | |
| stearic acid | |
| Capric acid | Am J. Physiol. 234(6): E 593, 1978 |
| Lauric acid | ″ |
| Palmitoleic acid | ″ |
| Linoleic acid | ″ |
| Arachidonic acid | ″ |
| N₂ Ac IleOH₃ | Biochemistry 19: 2616, 1980 |
| N₂ Ac LeuOCH₃ | ″ |
| 1,2-epoxy-3-(p-nitrophenoxy) | ″ |
| propane | |
| Sta Leu Phe | Nature 303: 81, 1983 |
| His Sta Leu Phe | ″ |
| SEQ ID NO:36 | |
| Phe His Sta Leu Phe | ″ |
| SEQ ID NO:37 | |
| Pro Phe His Sta Leu Phe | ″ |
| SEQ ID NO:38 | |
| Ibu His Pro Phe His Sta | ″ |
| SEQ ID NO:39 | |
| Ibu His Pro Phe His Sta Leu | ″ |
| SEQ ID NO:40 | |
| Ibu His Pro Phe His Sta Leu Phe | ″ |
| SEQ ID NO:41 | |
| Ibu His Pro Phe His Sta Ala Phe | ″ |
| SEQ ID NO:42 | |
| Ibu His Pro Phe His Sta Val Phe | ″ |
| SEQ ID NO:43 | |
| Ibu His Pro Phe His Sta Ile Phe | ″ |
| SEQ ID NO:44 | |
| Boc His Pro Phe His Sta Leu Tyr | ″ |
| SEQ ID NO:45 | |
| Boc His Pro Phe His Sta Leu | ″ |
| PheOCH₃ | |
| SEQ ID NO:46 | |
| Iva His Pro Phe His Sta Leu | ″ |
| Val Phe | |
| SEQ ID NO:47 | |
| POA Leu Sta Val PheOCH₃ | ″ |
| (POA equals phenoxyacetyl) | |
| SEQ ID NO:48 | |
| POA Leu Sta Leu PheOCH₃ | ″ |
| SEQ ID NO:49 | |
| Poa His Sta Leu PheOCH₃ | Nature 303: 81, 1983 |
| SEQ ID NO:50 | |
| Iva His Pro Phe His Leu | ″ |
| Sta Val Phe | |
| SEQ ID NO:51 | |
| Iva His Pro Phe His Sta | ″ |
| Leu Phe | |
| SEQ ID NO:52 | |
| BocPhe Phe Sta BmStaNH₂ | J. Chem. Soc. Chem. Commun: |
| | 109, 1985 |
| Naphthylalanyl His | Kobuku et al., Hypertension: |
| Sta4-amino-1-benzyl | 7(3): 837 (1985) |
| piperidine | |
| BNMA* His Sta2(S)methyl | Kobuku et al., Hypertension: |
| Butylamine | 7(3): 837 (1985) |
| BNMA Val Sta Isoleucinal | Kobuku et al., Hypertension: |
| | 7(3): 837 (1985) |
| 2,3-naphthylalanyl Leu | Kobuku et al., Hypertension: |
| Sta Isoleucinal | 7(3): 837 (1985) |
| BNMA Norleucinyl | Kobuku et al., Hypertension: |
| Isoleucinal | 7(3): 837 (1985) |

^{R}*indicates reduction of the amide carbonyl to a methylene.
*BNMA = betanaphthyl methyl analyl

The peptides can be synthesized by the well known solid phase peptide synthesis (Merrifield, R. B., J. Am. Chem. Soc. 85: 2149-2154 (1963), and Burton, Biochem. 14: 3892-3898 (1975)). On completion of chemical synthesis, the peptides can be deprotected and cleaved from the polymer by treatment with liquid HF-10% anisole for about 1 hour at 0 ° C. After evaporation of the reagents, the peptides can be extracted from the polymer with 1% acetic acid solution that is then lyophilized to yield the crude material. This crude material can be purified by such techniques as gel filtration on Sephadex G-15™ using 5% acetic acid as a solvent. Lyophilization of the appropriate fractions of the column eluate yield the homogeneous pentapeptide amides, which can be characterized by amino acid analysis, thin layer chromatography, high performance chromatography, ultraviolet absorption spectroscopy, molar rotation, solubility, and renin inhibitory potential.

Other agents that inhibit or modulate renin activity or mast cell degranulation can be used in the methods of the invention. Agents that inhibit renin expression or activity include small interfering RNAs (siRNAs), ribozymes, antisense nucleic acids, kinase inhibitors, anti-renin antibodies, small molecules, peptides, mutant renin polypeptides and the like.

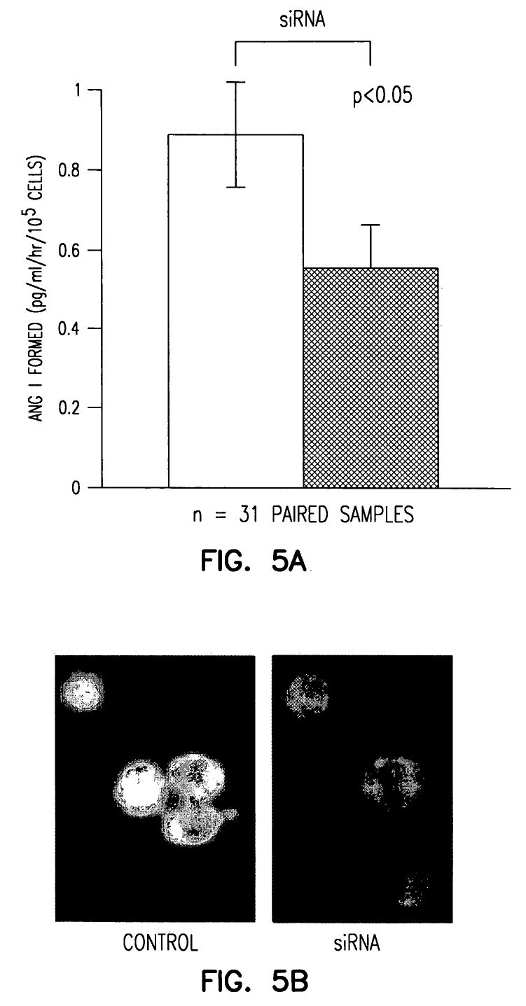

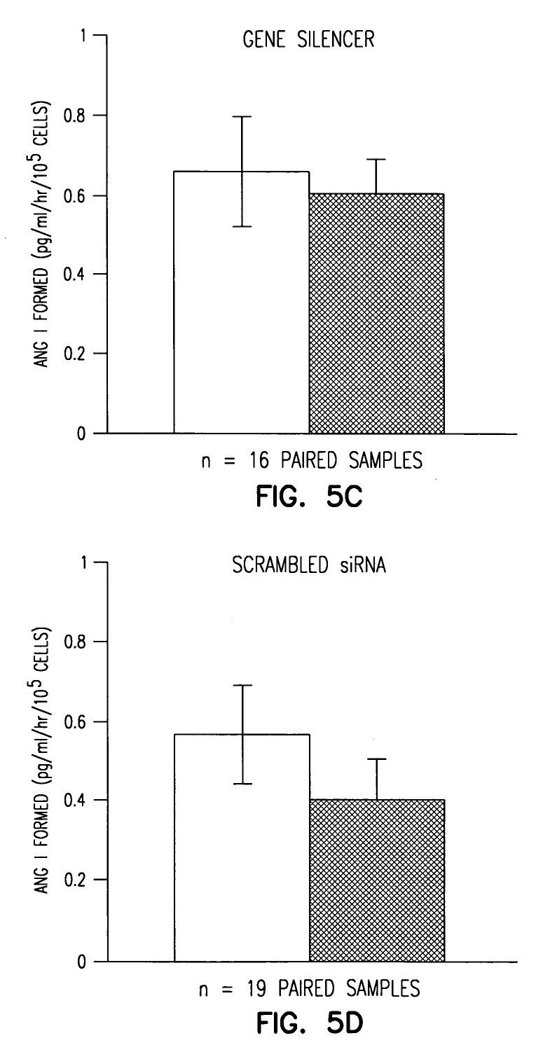

For example, small interfering RNAs (siRNA) targeted against renin transcripts were used to specifically reduce renin expression in HMC-1 mast cells (see FIG. 5). In the HMC-1 cells transfected with siRNA, there was an approximate 40% decrease in the absolute amount of angiotensin I formed compared to the amount measured from these cells before transfection. As shown in FIG. 5B, HMC-1 cells transfected with siRNA specific for renin display significantly reduced anti-renin immunostaining compared to control cells that did not receive the siRNA. These results indicate that synthesis of renin protein was considerably less in the cells exposed to the renin-specific siRNA.

Thus, in one embodiment, a condition involving an inappropriate mast cell-derived renin expression can be treated by administering to a mammal a nucleic acid that can inhibit the functioning of a renin RNA. Nucleic acids that can inhibit the function of a renin RNA can be generated from coding and non-coding regions of the renin gene. In the example provided herein, the renin-specific siRNA was based upon the coding sequence of human renin exon 2-8, available as NCBI accession number M26900 (gi: 337343). See website at ncbi.nlm.nih.gov. This sequence is provided below for easy reference (SEQ ID NO:53).

| | |
|---|---|
| 1 | ACAAGAAGTA ACTCTTATAA ATGCTCCAGA GGCCCTCAGT |
| 41 | GACAGAGGTG ATTTCCAGGT GGCTGGGCTA ACGTTAAAGG |
| 81 | TGGTTGTACT AAAGAGAGGG GTTTGGCCTC AGGGACTCAC |
| 121 | ATGTGGTGGA GGTACAGCAC TTTTCTATTT TTGCTTCCTC |
| 161 | CACCCTGGGC CAGGATCTTC CTCAAGAGAA TGCCCTCAAT |
| 201 | CCGAGAAAGC CTGAAGGAAC GAGGTGTGGA CATGGCCAGC |
| 241 | CTTGGTCCCG AGTGGAGCCA ACCCATGAAG AGGCTGACAC |
| 281 | TTGGCAACAC CACCTCCTCC GTGATCCTCA CCAACTACAT |
| 321 | GGACGTGAGT GCTTGGCTCA GCCCCTCGCT CCCTCCCTGT |
| 361 | CTCCTTTCCC TCATGGACCT AGGGCTTTCT TTGCTGCAAG |
| 401 | ACTCACCCTT TCCAAGCTGT GTTTGACGAA GGCGCTGAGT |
| 441 | AGCTGCAGGA AAATGGAAAC CCCGACAGGT ATAGGACCTC |
| 481 | GCCTGGGGCA AGTCTACACC CGAGAGCCAA GAGTGAAGCC |
| 521 | AGGCAAGACC CCAAGCCCAA GGTCCCCTGA GCCCCTCCAG |
| 561 | CCCTCTCTTT TTACCCCACA GACCCAGTAC TATGGCGAGA |
| 601 | TTGGGATCGG GACCCCACCC CAAACCTTCA AAGTCGTCTT |
| 641 | TGACACTGGT TCGTCCAATG TTTGGGTGCC CTCCTCCAAG |
| 681 | TGCAGCCGTC TCTACACTGC CTGTGGTGAG ACCTAAGACC |
| 721 | CACACTGCCT CTCCTCCATC CCCCTGCCCT ACTGTGCATG |
| 761 | AGCAATCCTG CCCAACACCC AGCTCCCATC CCTCTTGCCA |
| 801 | CCAAGGGAGT GGCTTCCTCT CTGCCTCTGT GCCCACTGAC |
| 841 | ATGTAGGGGA GAGGGGAAGA TGTCTCCCGT TTTTCTGATA |
| 881 | CAGCCACCAA GGTTAAAAAC AAAAAAAGGT CCAAGAACCC |
| 921 | CTGAGNACCC AGGAGGACCA GTTCCCAGTC GTCCTGAGAT |
| 961 | TGAGACAGGA CTGAATTCTC AAACCCATCC CAGGCACTCG |
| 1001 | GAACTCTTCC ATCCCTAGTC TTAATCAACA ACCTCTTACT |
| 1041 | AGCACTTACT CTGTGCCTGG CATACTTCTC TGGTGTTATC |
| 1081 | AGTGGTTAGT GATTACTTTA AATTCCTTCA TTTAGGACAA |
| 1121 | AATTCTCGAT GTATGGGACA CTTAGGAGAG CCCAAGAAAC |
| 1161 | CCAGTCCTTG ATTGATGAAG CACATATTCC AAGCCCCCTG |
| 1201 | ACCCTAGGGC CACTCATCCC TGCACCTAAG CTAACCAGCC |
| 1241 | ATACCCACAA TGCACCCTGC CTCTGAGTCC CCCTGTCTGG |
| 1281 | GCCACTCTTG GACAAACCTG AGCCTCTGTC CCCCTGCCAG |
| 1321 | TGTATCACAA GCTCTTCGAT GCTTCGGATT CCTCCAGCTA |
| 1361 | CAAGCACAAT GGAACAGAAC TCACCCTCCG CTATTCAACA |
| 1401 | GGGACAGTCA GTGGCTTTCT CAGCCAGGAC ATCATCACCG |
| 1441 | TAAGTTGGGC CGCCCTAGGT CATCTGCCCC GGACCCCTTC |
| 1481 | TGTCCCCAGG CCTCTCCTGA CCCACCAGGG CCCACACCTG |
| 1521 | CGGGGAGGTA CACTGCAGCC CACTTGGAGC CTGGGGAGCT |
| 1561 | GAGGAACACC CTACTCTGCC ACATCTGGTG TTGAAAGCAG |
| 1601 | CAGTACCTAT GGGGGAGCAA GCCTGGGCTA CGGGCTCACC |
| 1641 | GTTGGGTGGT TTGTGGATGT TTTTGCATCT AACTTGCATG |
| 1681 | TAGGGCTGTC CTGAGCCCCG TGGCTGCAGT CAAGTAACTC |
| 1721 | GTCCCAAGTT CACCAGCTCT GACTGGGGCT ACTACCCTAG |
| 1761 | ACTGAAATCC TGGGTCAGAG TCAGGCTATT TTAGGGTCAG |
| 1801 | GCATAGTTTT AAGGTCACAT TAGTTGACTC TGGGACTCAG |
| 1841 | GTCAAGGCTC TCTTTTCTTT TCCATGTGGC CCATGTCTGA |
| 1881 | CCGTTTCCTC ATCCTGGAGT TTCTCAGGCC CTGCTCCATC |
| 1921 | AGAGTTAGGG GAGGGGCACA CGTGGCACCT GAGAGGAAAT |
| 2061 | CAGGGTGATT CCTGCCTCCC TTCCTTTTTC TGTTGAACTC |
| 2101 | TGATATAAAG GAGGAAGAAG GGCAAGCTTG TCTGTGCTAA |
| 2041 | AGAAACCCTT CGCCCATGAT AAGGGTGGGG CCAAGACCCA |
| 2081 | GTCCTGCCAG GCACGAAAGT CTGGCCACTG GGGAGGGGAG |
| 2121 | GAGCTCTTGG ACTTTTCTTT TGCGCTTGGC AGGACCACCC |
| 2161 | TCTCAGCCTC TGCTCTCCGA TCCCTGGTCA ACTCTAGCTC |
| 2201 | TCTCTGGGCT CCGCAGCAGA GATGTGTATT GGCACAGAGT |
| 2241 | GTGTGCGTGC AGGGTTGAGG CAATACTCTT ACCCCGATTT |
| 2281 | CTGTACCCTG GAGCATGTGT GCCCCTGGGA TCCCTAGTGT |
| 2321 | GGATGCCCAG ACCAGACTCC AACCAAGGAG GGGCAGTGGG |
| 2361 | CTTGGTCTCC TATGGTCCTT CCTCCCACAG GTGGGTGGAA |
| 2401 | TCACGGTGAC ACAGATGTTT GGAGAGGTCA CGGAGATGCC |
| 2441 | CGCCTTACCC TTCATGCTGG CCCAGTTTGA TGGGGTTGTG |
| 2481 | GGCATGGGCT TCATTGAACA GGCCATTGGC AGGGTCACCC |
| 2521 | CTATCTTCGA CAACATCATC TCCCAAGGGG TGCTAAAAGA |
| 2561 | GGACGTCTTC TCTTTCTACT ACAACAGGTG GGGACTGGGA |
| 2601 | CTCCAAGGGC TGAGGTGGGG GGCAGGAGGG GAGAAGAGAT |
| 2641 | GGGGACTGGA AGGAGAGTCT GGGCCAGAAT TGTAAAGTGT |
| 2681 | TTGTAACTTA GGTGACAGCC AATCAATATC TAGAGCTGTA |
| 2721 | CTAGCCAATA TGGAAGGCAC TATTGCAAAT TTAAACTTAA |
| 2761 | CTTAAATACA GCTTAAGCAT CAATTAAGCA TTCAACTGGC |
| 2801 | TGGCCTCTTA GTTGTACTAG CCACAGCTCA ATGCCTGGCA |
| 2841 | GCCACGGTGG CTAGTAACTA CAGTCTAGTA CAGTGCAGAT |
| 2881 | AGAGATATCC AGCATGACAG GACATCTATA GACAGCGCCA |
| 2921 | CTAAAAGAAT AGAGGAGGAT CAGAGTTCAG AGAAATCTCA |
| 2961 | CAGTAAAATG GAGAGGAGTC TCCGGTTTGG TGATAGAAAG |
| 3001 | TGAGGCCTTG AGAAAAGGCC AATTGGCGGC TCTGCATTCA |
| 3041 | GGGGTGGTCT TTAGAAGAAC TGTTTTAGAG GAGGTGGGGG |
| 3081 | CAAGGCCAGA TGGCAAGAAG TTAAGAGGTG GACGACGTGG |
| 3121 | GTGTCAGGAA GTGGAGGTCA TGAGATGTAC GCTGCCCTGG |
| 3161 | GACATTCAAC AGGGAAGGGA ATGGGGGGTG GCGTGGGGGG |
| 3201 | GTGAGATCCA GAAGCAGAAG AGGAAGGGTG GGTGTTTTTA |
| 3241 | AATGCTAGAG GATGCTCGAG TGATCGCCTG TAGGTGGAGG |
| 3281 | AAGAACCCAA TAGAAAGAAA GAGATTAAAA ATGTGGAAAG |
| 3321 | AAGAGGAGCT AAATGGGGGC ACTGGAGTTT AGAGGCCTTG |
| 3361 | AAAGAGATGA GGAACCAGCA GATAGGAAGA AGCCAGGTTT |
| 3401 | TACAGAGGAG AGGGCTGGCC TCTTCTTTTA TCTTGGGATG |
| 3441 | GGAAGGAGGG AACATCCAGA GAGATACTGA AGTGTTGAGA |
| 3481 | GACAGGCAGG AGGGAATTTG TGCTAGCATA TACACATACG |
| 3521 | AGTTCCGAAT TTATAAAAAC ACAAGTAGTT TGCAGTTGCA |
| 3561 | CAAAATAACA TATGCACACC TACACACCCA TGCACACATG |
| 3601 | TGCATGTGAA TTCTGGAAAA ACACATCACA CACACAGGCA |
| 3641 | TGCCCTGGAG ACTAGGCCTA CAGTAGTCCC TGAGCCAAGT |
| 3681 | GCAGTGAGGA GGAAAGGAAG GTGAGGGGAA TCATCTCCAG |
| 3721 | ACGGGGCACC AGGAGCCTGG CTCCAGTCCC CCACTTGTTC |
| 3761 | ACTCATGGAC TGGGTAACTT CAGGCAAGTG ACTTCGCCTC |
| 3801 | TTGGTGACTC CATTGCCTGA AGGGCAAAGA GAGTACATAA |
| 3841 | CACCCACCCT GCCAAACAGC AGGGTGATGA GGCTGGCATG |
| 3881 | AAATGAAGCT TCCTTTCTGC TGTCTCTCTT TCTCTGCAGA |
| 3921 | GATTCCGAGT AAGGAGACAA AACCCCCACA TGGCTGTGAC |
| 3961 | CTTCCAGTAT TCCCCGAGCA CCTGACCTAG AATTACACAC |
| 4001 | GCCACCGGCC CAAAACTCAC ATCAGCAAGT CCCAGCCTCC |
| 4041 | GCTAGATGCC GAAGTTCTCT GTCTCTCCTT CCTGCTCTCT |
| 4081 | CCATGCCACC TGCCCACCCC ATACCCAATA GCCTCCCCAG |
| 4121 | GGTCCCCTCC CATGCACCTG CTCAATCAGC AGCAACCCAA |
| 4161 | GAGTGAGGGG TGTCCATTTG TGTCTTGTTC ACATCCACTC |
| 4201 | ACTGTCCTTG TACCTGCTCC TTTTCTGTGA CCTCTCTGGG |
| 4241 | GATGCTTTTT GGGGGAACAG CTGGACTACC CTGGAACAAC |
| 4281 | CTCTGGTTGG TCTTGGGGAG GGGAAGAAAG GCAGAGAAGC |
| 4321 | AGTATGTTCT GCATGCTTCC CAACGACAGC TCCGAGCCTG |
| 4361 | GCTGTCTGTC CCACATTCCT CTGCTCTAGA GCCCTCTGTC |
| 4401 | CTCCCCTCGA CCCTTGTGCA ACCTTCCCCA ATTGCCTGAG |
| 4441 | TTGCTGGGTC CTGGAGGTTA TGGGTTTCCA AGAGCTTCTG |
| 4481 | ATCTTTCCTT TAGGAATTCC CAATCGCTGG GAGGACAGAT |
| 4521 | TGTGCTGGGA GGCAGCGACC CCCAGCATTA CGAAGGGAAT |
| 4561 | TTCCACTATA TCAACCTCAT CAAGACTGGT GTCTGGCAGA |
| 4601 | TTCAAATGAA GGGGTCAGAA ATCCTCAGAC CCTCCCCGGG |
| 4641 | CTCCAAAAAA TGCTGCCGTC ACTGGGGTTG GGGAGGGCGG |
| 4681 | GCGCGGACTG CATTACCATC CTGCCCTCTT TCCAAATGCA |
| 4721 | GCCACTTCTT AAGCACAGCC ACCATTTGCT CTCTGCCTGG |
| 4761 | CTCTGGTCCA GGCTGGGGCA GAGAGAAGGG AGGGGCCTGG |
| 4801 | GCCGGAGTGG TGGAGGCCGA GAGTACCTTC CCTCCTCTAC |
| 4841 | TCACTGCCTC AACAGCCAGC CAGCGTGGCG CTCCACCCAC |
| 4881 | CCACCCACCA CTCAGGAAGG ACATGCAGCC TGGCGTGCCC |
| 4921 | ATCAGCCTTC TGTCTGTCTG TCTGTCTGTC TGTCTCTCTG |
| 4961 | TCTGACTGTG GCGCTCCCCC AGGGTGTCTG TGGGGTCATC |
| 5001 | CACCTTGCTC TGTGAAGACG GCTGCCTGGC ATTGGTAGAC |
| 5041 | ACCGGTGCAT CCTACATCTC AGGTTCTACC AGCTGCATAG |
| 5081 | AGAAGCTCAT GGAGGCCTTG GGAGCCAAGA AGAGGCTGTT |
| 5121 | TGATGTAAGA AGCCAAAGAG GGAAGGTGCT GTGGGTGTGG |
| 5161 | GGAGCGGCCA CCTGGTATCG GCTCACAAAT CCCCCAGGCA |
| 5201 | AATGAGGCCA TCTCAGGCCT TCGCTTGTTC ACCTCACACT |
| 5241 | CTCCACACAT GTGGCTGGTC ACCCATGGGG CGGGGCACTG |
| 5281 | TCCCCAGCCC TCTCCAGCAG AGAGACCCAG GGCCACCAGC |
| 5321 | GCAGGACTCC TTGTCTGCTG AGACGTCGTT CCATACTCAA |
| 5361 | GAAGGCTCTC TTTGCCCCCC ACCCCAGTAT GTCGTGAAGT |
| 5401 | GTAACGAGGG CCCTACACTC CCCGACATCT CTTTCCACCT |
| 5441 | GGGAGGCAAA GAATACACGC TCACCAGCGC GGACTATGTA |
| 5481 | TTTCAGGTGA GGTTCGAGTC GGCCCCCTCG GTGGCAGGGA |
| 5521 | GAAAGGCTGG ACAGAGACCC TCAAGAGTGA CAGATTACAA |
| 5561 | TGCACAGATC ATGTTAGAAC TGTAGTTCTC AAACTTGGCT |
| 5601 | GTGCATGTCA CCTGGAGAGC TTTGGAAAAA TCCAGGTACC |
| 5641 | TGGGCCACAT CCCATACCTA TTAAATCAGA ACCTCTAGAA |
| 5681 | GTGGGACCTG GGGTTCAGTT TCCCCAGATG ATTCCAATGT |
| 5721 | GTGGCCATGT TTGGGCATCA CTATGCCTGT TCCCTCATCT |
| 5761 | CCATTTTCTC ATCAAATACT CCCAATAATC CTATGCTCCT |
| 5801 | ATATTCTTAC CCTCTTTTCA TAATCAATAG GCTTAGAGAA |
| 5841 | TTTGAATAAC TTGTCTAGGA TCAGAAGCTA AGGCAAACTG |
| 5881 | TAAGCTCCTG AAGGAAGCAC GTTGCCTGAT GCCCTGTTTG |
| 5921 | CCTGGGATCT AGCACAGGGG CTAAACATAG GAATGGTGCA |
| 5961 | GTCCACGATG GGGCAAAAT |

However, nucleic acids that can inhibit the function of a renin RNA can be selected from other regions of the renin RNA. For example, one sequence for a human renin cDNA can be found in the NCBI database at accession number NM 000537 (gi: 11125774). This sequence is provided below for easy reference (SEQ ID NO:54).

| | |
|---|---|
| 1 | AGAACCTCAG TGGATCTCAG AGAGAGCCCC AGACTGAGGG |
| 41 | AAGCATGGAT GGATGGAGAA GGATGCCTCG CTGGGGACTG |
| 81 | CTGCTGCTGC TCTGGGGCTC CTGTACCTTT GGTCTCCCGA |
| 121 | CAGACACCAC CACCTTTAAA CGGATCTTCC TCAAGAGAAT |
| 161 | GCCCTCAATC CGAGAAAGCC TGAAGGAACG AGGTGTGGAC |
| 201 | ATGGCCAGGC TTGGTCCCGA GTGGAGCCAA CCCATGAAGA |
| 241 | GGCTGACACT TGGCAACACC ACCTCCTCCG TGATCCTCAC |
| 281 | CAACTACATG GACACCCAGT ACTATGGCGA GATTGGGATC |
| 321 | GGGACCCCAC CCCAAACCTT CAAAGTCGTC TTTGACACTG |
| 361 | GTTCGTCCAA TGTTTGGGTG CCCTCCTCCA AGTGCAGCCG |
| 401 | TCTCTACACT GCCTGTGTGT ATCACAAGCT CTTCGATGCT |
| 441 | TCGGATTCCT CCAGCTACAA GCACAATGGA ACAGAACTCA |
| 481 | CCCTCCGCTA TTCAACAGGG ACAGTCAGTG GCTTTCTCAG |
| 521 | CCAGGACATC ATCACCGTGG GTGGAATCAC GGTGACACAG |
| 561 | ATGTTTGGAG AGGTCACGGA GATGCCCGCC TTACCCTTCA |
| 601 | TGCTGGCCGA GTTTGATGGG GTTGTGGGCA TGGGCTTCAT |
| 641 | TGAACAGGCC ATTGGCAGGG TCACCCCTAT CTTCGACAAC |
| 681 | ATCATCTCCC AAGGGGTGCT AAAAGAGGAC GTCTTCTCTT |
| 721 | TCTACTACAA CAGAGATTCC GAGAATTCCC AATCGCTGGG |
| 761 | AGGACAGATT GTGCTGGGAG GCAGCGACCC CCAGCATTAC |
| 801 | GAAGGGAATT TCCACTATAT CAACCTCATC AAGACTGGTG |
| 841 | TCTGGCAGAT TCAAATGAAG GGGGTGTCTG TGGGGTCATC |
| 881 | CACCTTGCTC TGTGAAGACG GCTGCCTGGC ATTGGTAGAC |
| 921 | ACCGGTGCAT CCTACATCTC AGGTTCTACC AGCTCCATAG |
| 961 | AGAAGCTCAT GGAGGCCTTG GGAGCCAAGA AGAGGCTGTT |
| 1001 | TGATTATGTC GTGAAGTGTA ACGAGGGCCC TACACTCCCC |
| 1041 | GACATCTCTT TCCACCTGGG AGGCAAAGAA TACACGCTCA |
| 1081 | CCAGCGCGGA CTATGTATTT CAGGAATCCT ACAGTAGTAA |
| 1121 | AAAGCTGTGC ACACTGGCCA TCCACGCCAT GGATATCCCG |
| 1161 | CCACCCACTG GACCCACCTG GGCCCTGGGG GCCACCTTCA |
| 1201 | TCCGAAAGTT CTACACAGAG TTTGATCGGC GTAACAACCG |
| 1241 | CATTGGCTTC GCCTTGGCCC GCTGAGGCCC TCTGCCACCC |
| 1281 | AGGCAGGCCC TGCCTTCAGC CCTGGCCCAG AGCTGGAACA |
| 1321 | CTCTCTGAGA TGCCCCTCTG CCTGGGCTTA TGCCCTCAGA |
| 1361 | TGGAGACATT GGATGTGGAG CTCCTGCTGG ATGCGTGCCC |
| 1401 | TGACCCCTGC ACCAGCCCTT CCCTGCTTTG AGGACAAAGA |
| 1441 | GAATAAAGAC TTCATGTTCA C |

In some embodiments, the nucleic acid that can inhibit the function of a renin RNA can be complementary to sequences near the 5′ end of the renin coding region.

Hence, in some embodiments, the nucleic acid that can inhibit the functioning of a renin RNA can be complementary to SEQ ID NO:53 or 54. In other embodiments, nucleic acids that can inhibit the function of a renin RNA having SEQ ID NO:54 can be complementary to the 5′ ends of SEQ ID NO:53, SEQ ID NO:54 or to renin RNAs from other species (e.g., mouse, rat, cat, dog, goat, pig or a monkey renin RNA).

A nucleic acid that can inhibit the functioning of a renin RNA need not be 100% complementary to a selected region of SEQ ID NO:53 or 54. Instead, some variability the sequence of the nucleic acid that can inhibit the functioning of a renin RNA is permitted. For example, a nucleic acid that can inhibit the functioning of a human renin RNA can be complementary to a nucleic acid encoding a mouse or rat renin gene product. Nucleic acids encoding mouse renin gene product, for example, can be found in the NCBI database.

Moreover, nucleic acids that can hybridize under moderately or highly stringent hybridization conditions are sufficiently complementary to inhibit the functioning of a renin RNA and can be utilized in the compositions of the invention. Generally, stringent hybridization conditions are selected to be about 5° C. lower than the thermal melting point (Tₘ) for the specific sequence at a defined ionic strength and pH. However, stringent conditions encompass temperatures in the range of about 1° C. to about 20° C. lower than the thermal pointing point of the selected sequence, depending upon the desired degree of stringency as otherwise qualified herein. In some embodiments, the nucleic acids that can inhibit the functioning of renin RNA can hybridize to a renin RNA under physiological conditions, for example, physiological temperatures and salt concentrations.

Precise complementarity is therefore not required for successful duplex formation between a nucleic acid that can inhibit a renin RNA and the complementary coding sequence of a renin RNA. ihibitory nucleic acid molecules that comprise, for example, 2, 3, 4, or 5 or more stretches of contiguous nucleotides that are precisely complementary to a renin coding sequence, each separated by a stretch of contiguous nucleotides that are not complementary to adjacent renin coding sequences, can inhibit the function of renin mRNA. In general, each stretch of contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of a nucleic acid hybridized to a sense nucleic acid to estimate the degree of mismatching that will be tolerated between a particular nucleic acid for inhibiting expression of a particular renin RNA.

In some embodiments a nucleic acid that can inhibit the function of an endogenous renin RNA is an anti-sense oligonucleotide. The anti-sense oligonucleotide is complementary to at least a portion of the coding sequence of a gene comprising SEQ ID NO:53 or 54. Such anti-sense oligonucleotides are generally at least six nucleotides in length, but can be about 8, 12, 15, 20, 25, 30, 35, 40, 45, or 50 nucleotides long. Longer oligonucleotides can also be used. renin anti-sense oligonucleotides can be provided in a DNA construct, or expression cassette and introduced into cells whose division is to be decreased, for example, into cells expressing renin, including mast cells.

In one embodiment of the invention, expression of a renin gene is decreased using a ribozyme. A ribozyme is an RNA molecule with catalytic activity. See, e.g., Cech, 1987, Science 236: 1532-1539; Cech, 1990, Ann. Rev. Biochem. 59:543-568; Cech, 1992, Curr. Opin. Struct. Biol. 2: 605-609; Couture and Stinchcomb, 1996, Trends Genet. 12: 510-515. Ribozymes can be used to inhibit gene function by cleaving an RNA sequence, as is known in the art (see, e.g., Haseloff et al., U.S. Pat. No. 5,641,673).

Nucleic acids complementary to SEQ ID NO:53 or 54 can be used to generate ribozymes that will specifically bind to mRNA transcribed from a renin gene. Methods of designing and constructing ribozymes that can cleave other RNA molecules in trans in a highly sequence specific manner have been developed and described in the art (see Haseloff et al. (1988), Nature 334:585-591). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201). The target sequence can be a segment of about 10, 12, 15, 20, or 50 contiguous nucleotides selected from a nucleotide sequence having SEQ ID NO:53 or 54. Longer complementary sequences can be used to increase the affinity of the hybridization sequence for the target. The hybridizing and cleavage regions of the ribozyme can be integrally related; thus, upon hybridizing to the target RNA through the complementary regions, the catalytic region of the ribozyme can cleave the target.

RNA interference (RNAi) involves post-transcriptional gene silencing (PTGS) induced by the direct introduction of dsRNA. Small interfering RNAs (siRNAs) are generally 21-23 nucleotide dsRNAs that mediate post-transcriptional gene silencing. Introduction of siRNAs can induce post-transcriptional gene silencing in mammalian cells. siRNAs can also be produced in vivo by cleavage of dsRNA introduced directly or via a transgene or virus. Amplification by an RNA-dependent RNA polymerase may occur in some organisms. siRNAs are incorporated into the RNA-induced silencing complex, guiding the complex to the homologous endogenous mRNA where the complex cleaves the transcript.

Rules for designing siRNAs are available. See, e.g., Elbashir SM, Harborth J, Lendeckel W, Yalcin A, Weber K, Tuschl T (2001). Duplexes of 21-nucleotide RNAs mediate RNA interference in mammalian cell culture. Nature 411: 494-498; J. Harborth, S. M. Elbashir, K. Vandenburgh, H. Manninga, S. A. Scaringe, K. Weber and T. Tuschl (2003). Sequence, chemical, and structural variation of small interfering RNAs and short hairpin RNAs and the effect on mammalian gene silencing, Antisense Nucleic Acid Drug Dev. 13: 83-106.

Thus, an effective siRNA can be made by selecting target sites within SEQ ID NO:53 or 54 that begin with AA, that have 3′ UU overhangs for both the sense and antisense siRNA strands, and that have an approximate 50% G/C content. For example, a siRNA of the invention that can hybridize to renin nucleic acids can be complementary to one of the following sequences:

| | |
|---|---|
| AACCTCAGTG GATCTCAGAG AAA | (SEQ ID NO:55) |
| AAGCATGGAT GGATGGAGAA GAA | (SEQ ID NO:56) |
| AAGGATGCCT CGCTGGGGAC AA | (SEQ ID NO:57) |

Actual siRNAs used to inhibit the functioning of renin mRNA had the following sequences:

| | |
|---|---|
| GAGAAAGGCTGGACAGAGA | (SEQ ID NO:58) |
| TCAACTGGCTGGCCTCTTA | (SEQ ID NO:59) |
| GTACAGCACTTTTCTATTT | (SEQ ID NO:60) |
| GCAAAGAGAGTACATAACA | (SEQ ID NO:61) |

Thus, nucleic acids that can decrease renin expression or translation can hybridize to a nucleic acid comprising SEQ ID NO:53 or 54 under physiological conditions. In other embodiments, these nucleic acids can hybridize to a nucleic acid comprising SEQ ID NO:53 or 54 under stringent hybridization conditions. Examples of nucleic acids that can modulate the expression or translation of a renin polypeptide include a siRNA that consists essentially of an nucleic acid (e.g. an RNA) complementary to any one of SEQ ID NO:55, 56 or 57. Further examples of nucleic acids that can modulate the expression or translation of a renin polypeptide include a siRNA that consists essentially of an nucleic acid (e.g. an RNA) having any one of SEQ ID NO:58-61.

Mast Cell Stabilizers

Mast cells are a normal component of the connective tissue and play an important role in immediate (type I) hypersensitivity and inflammatory reactions by secreting a large variety of chemical mediators from storage sites in their granules upon stimulation. As described herein, one of the chemical mediators secreted by mast cells is renin.

According to the invention, any mast cell stabilizer can be used to inhibit renin release. Examples include lodoxamide, cromolyn sodium, nedocromil, nicardipine, barnidipine, YC-114, elgodipine, niguldipine and R(−)-niguldipine. Dihydropyridines, such as nicardipine and nifedipine have been shown to inhibit histamine release from human lung and tonsillar cells (Kim et al., Inhibition of Histamine Release from Dispersed Human Lung and Tonsillar Mast Cells by Nicardipine and Nifedipine, British Journal of Clinical Pharmacology, volume 19, pages 631-638 (1985)).

Some data exists indicating that mast cell populations of various tissues within the same species may differ in phenotype, biochemical properties, functional and pharmacological responses and ontogeny. Hence, mast cells are heterogeneous. See, for example, Irani et al., Mast Cell Heterogeneity, Clinical and Experimental Allergy, volume 19, pages 143-155 (1989). Because different mast cells may exhibit different responses to pharmacological agents, one of skill in the art may not be able to use compounds claimed to be anti-allergic (“mast cell stabilizers”) in all mast cell populations.

As shown for the first time herein, renin can be produced locally. Hence, localized administration of mast cell stabilizers and/or renin inhibitors is appropriate. Such localized administration is preferred because mast cell and renin functioning in other organ systems will be substantially unaffected. Hence, the invention contemplates targeting the tissue or organ adversely affected by high levels of renin or angiotensin activity in a localized manner to control such adverse affects without significantly influencing unaffected tissues and organs.

Agents for Blocking Sodium—Hydrogen Exchangers

The invention further contemplates administering compounds that inhibit the sodium/hydrogen exchange type-1 (NHE-1) transport system. A number of NHE-1 inhibitors are available. For example, U.S. Pat. No. 6,423,705; EPO 0 918 515; CA 2,227,112; CA 2,245,776; and WO 99/43663 disclose certain NHE-1 inhibitors.

Agents for Blocking Angiotensin Type 1 Receptor (AT₁R) Activity

The invention further contemplates administering compounds that inhibit angiotensin type 1 receptor activity. A number of angiotensin type 1 receptor inhibitors are available. For example, Diovan™ (valsartan), Benicar™ (olmesartan), Atacand™ (candesartan), Avapro® (irbesartan), Cozaar® (losartan), Micardis® (telmisartan) or other available inhibitors of angiotensin type 1 receptor activity can be used. Diovan™ is a nonpeptide that can be obtained from Novartis. Benicar™ (olmesartan) can be obtained from Sankyo Pharma Inc. Atacand™ (candesartan) can be obtained from AstraZeneca. Avapro® (irbesartan) can be obtained from Bristol-Myers Squibb Company. Cozaar® (losartan) can be obtained from Merck & Co. Micardis® (telmisartan) can be obtained from Boehringer Ingelheim.

Cardiac Conditions

Ischemic heart disease, the number 1 cause of death worldwide, is responsible for approximately 14% of all deaths. Approximately 1.5 million Americans will have a heart attack this year as a result of myocardial ischemia, and approximately 500,000 of those will be fatal. Myocardial ischemia is the principal cause of heart failure in the Western world, accounting for 50-80% of all cases. In contrast, hypertension accounts for less than 50% of all cases of congestive heart failure. Myocardial ischemia is a major factor in cardiac remodeling, a process that changes the shape and size of the heart, and significantly worsens the prognosis of patients with heart failure. Myocardial ischemia is also associated with excessive release of catecholamines (norepinephrine) which leads to severe and often fatal arrhythmias, as seen in heart failure and sudden cardiac death. Mast cell hyperplasia is associated with myocardial infarction and atherosclerosis, two syndromes associated with myocardial ischemia. According to our invention, release of renin from cardiac mast cells in ischemia, drives the local formation of ANG II in the heart. ANG II is known to facilitate release of norepinephrine from sympathetic nerve endings leading to arrhythmias and stimulate cardiac hypertrophy, which contributes to the decreased contractility of the heart in heart failure. This source of extra-renal renin, the release of which leads to local ANG II formation, has never before been contemplated.

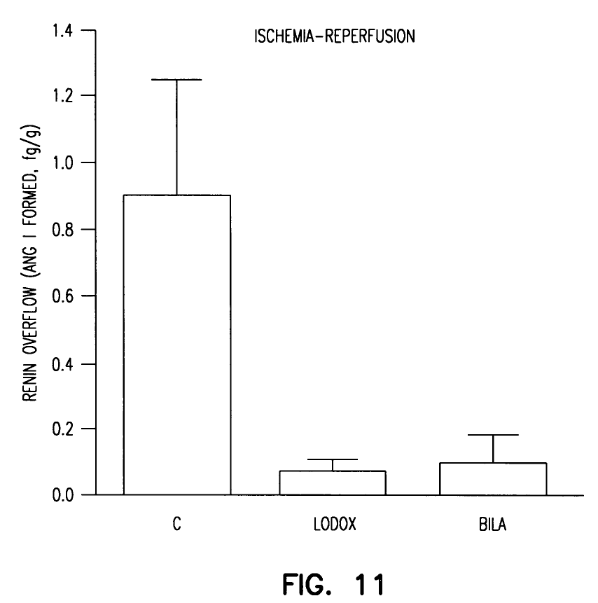

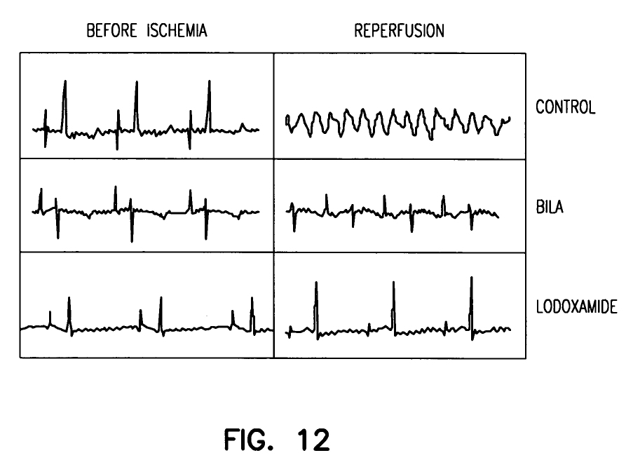

According to the invention, locally produced ANG II resulting from renin released from cardiac mast cells as occurs in response to ischemia and/or oxygen radical formation, exacerbates certain cardiac conditions, including sudden cardiac death, congestive heart failure, arrhythmias, and hypertension. Results provided herein (FIGS. 11-17) indicate that when isolated Langendorff hearts subjected to ischemia-reperfusion, the amount of renin and NE in the coronary effluent after ischemia reperfusion, is significantly increased, compared to pre-ischemic levels. Notably, no such increase occurs if Langendorff hearts are pre-treated with lodoxamide, a mast cell stabilizer, during ischemia/reperfusion suggesting that the renin measured in the coronary effluent of these hearts, is entirely mast-cell-derived. In addition the presence of a renin inhibitor blocks induction of ischemia-induced ventricular fibrillations (FIG. 12).

According to the invention, cardiac mast cells release renin and such release can exacerbate certain cardiac conditions, including congestive heart failure, ischemia and hypertension. Accordingly, it is an object of this invention to provide a method for treatment of patients with ischemia, congestive heart failure, or hypertension. The method involves administering to the patient a stabilizer of mast cells, an inhibitor renin expression, an inhibitor of renin activity or a similar agent that can prevent the detrimental effects of localized renin secretion.

It is another object of this invention to provide a method for treatment of patients with congestive heart failure, ischemia or hypertension, the method involving treating the patients with an effective amount of a renin inhibitor. The administration of mast cell stabilizers and/or renin inhibitors produces improvement of cardiac performance by increased ventricular contractility and decreased peripheral vascular resistance.

As mentioned above, localized administration of therapeutic agents is appropriate for targeting tissues or organs adversely affected by high levels of renin or angiotensin activity. Hence, the invention contemplates administration of mast cell inhibitors, renin inhibitors, nucleic acids that inhibit the function of renin RNA, ACE inhibitors, AT₁ receptor antagonists or modulators of the Na⁺/H⁺ exchanger (NHE) in a localized manner to prevent or treat cardiac conditions without significantly influencing unaffected tissues and organs.

Lung Conditions

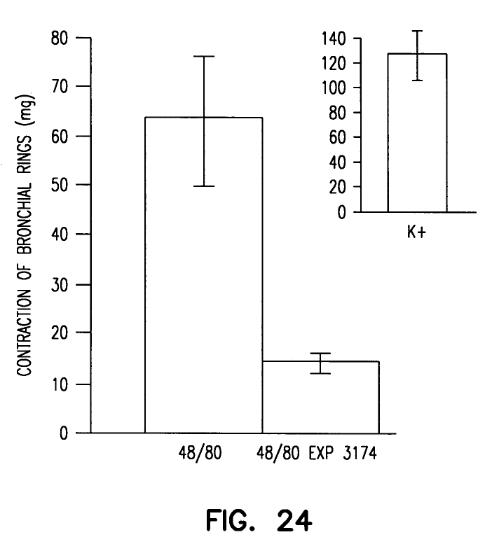

As mentioned above, mast cells play an important role in immediate (type I) hypersensitivity and inflammatory reactions by secreting a large variety of chemical mediators from storage sites in their granules, including renin. As shown in FIG. 23, renin-positive mast cells exist in the airways. According to our invention, this novel source of renin is released upon mast cell degranulation and mediates local formation of angiotensin II. Results presented in FIG. 24, support this claim and demonstrate that mast cell degranulation leads to contraction of smooth muscle in the bronchus. This contraction can be significantly inhibited in the presence of the ANG II ATₗR inhibitor, EXP 3174.

The event that initiates immediate hypersensitivity is the binding of antigen to IgE on the mast cell or basophil surface. Both cell types are activated by cross-linking of FcεRI molecules, which is thought to occur by binding multivalent antigens to the attached IgE molecules. Mast cells may also be activated by mechanisms other than cross-linking FcεRI, such as in response to mononuclear phagocyte-derived chemocytokines, to T cell-derived cytokines and to complement-derived anaphylatoxins. Mast cells may also be recruited and activated by other inflammatory cells or by neurotransmitters which serves as links to the nervous system.

When antigen binds to IgE molecules attached to the surface of mast cells, a variety of mediators are released that give rise to increased vascular permeation, vasodilation, bronchial and visceral smooth muscle contraction, and local inflammation. The most extreme form of immediate hypersensitivity reaction is known as anaphylaxis. During anaphylaxis, mediators released from mast cells can restrict airways to the point of asphyxiation. So-called atopic individuals, who are prone to develop strong immediate hypersensitivity responses, may suffer from asthma, hay fever or chronic eczema. These individuals possess higher than average plasma IgE levels.

Antigens that elicit strong immediate hypersensitivity reactions are known as allergens. Allergy afflicts twenty percent of the United States population. Immediate hypersensitivity results from the following sequence of events: production of IgE by B cells in response to antigen, binding of the IgE to FcεRI on the surface of mast cells, interaction of re-introduced antigen with the bound IgE and activation of the mast cells and release of mediators. Antigen binding can be simulated by polyvalent anti-IgE or by anti-FcεRI antibodies. Such antibodies can activate mast cells from atopic as well as non-atopic individuals, whereas allergens activate mast cells only in atopic persons.

According to the invention, mediators released from human mast cells are central to the pathophysiology of allergy, asthma and anaphylaxis. In particular, mast cells and their release of renin, histamine and other mediators play an important role in the symptomatology of asthma and other human diseases. During the early phase of human lung hypersensitivity reactions upon exposure to antigen (i.e., pollens, cats, etc.), mast cells release and are the major source of histamine, renin and newly synthesized lipid products of arachidonic acid metabolism: prostaglandin D₂ and leukotriene C₄. These mediators produce immediate breathlessness, which subsides in one hour but returns within 2-4 hours (the “late phase” response). Attesting to their primal role in hypersensitivity responses, human lung mast cells (HLMC) are characterized by MRNA generation, protein synthesis and release of so-called TH 2 cytokines within these first few hours of activation. These cytokines including IL-5, and IL-13 are believed to be central to the evolution of chronic allergic/asthmatic states. In the lung, mast cells are the only source of histamine. Thus, histamine release is a distinct marker of mast cell activation and behavior. For a review of the role of mast cells in inflammatory responses in the lung, see Schulman, Critical Reviews in Immunology, 13(1):35-70 (1993), the entire disclosure of which is incorporated herein by reference.

Clinically, asthma is recognized by airway hyperactivity and reversible airways obstruction. Pathological derangements at the tissue level include constriction of airway smooth muscle, increased vascular permeability resulting in edema of airways, outpouring of mucus from goblet cells and mucus glands, parasympathetic nervous system activation, denudation of airway epithelial lining cells, and influx of inflammatory cells. Underlying these tissue effects are direct effects of potent mediators secreted following physical, inflammatory, or immunological activation and degranulation. The early phase of the asthmatic reaction is mediated by histamine and other mast cell mediators that induce rapid effects on target organs, particularly smooth muscle.

The pathophysiological sequence of asthma may be initiated by mast cell activation in response to allergen binding to IgE. Evidence also exists to link exercise-induced asthma and so-called “aspirin-sensitive” asthma to human lung mast cell degranulation.

A number of pharmacologic agents have been tested for effect on human lung mast cell activation-secretion. The beta-adrenergic agonist pharmacologic agents, as typified by fenoterol, are the most potent global inhibitors of human lung mast cells. Though widely touted as “mast cell stabilizers,” disodium cromoglycate and nedocromil sodium poorly inhibit purified human lung mast cell histamine release. While certain corticosteroids have been found to suppress IgE-mediated generation of late-phase cytokine MRNA and protein (e.g., IL-5), release of early phase mediators (e.g., histamine and LTC₄) are unaffected by corticosteroids. Human lung mast cell release has been shown to be inhibited by the immunosuppressant agents FK-506, cyclosporin A and auranofin. Arachidonate pathway inhibitors are of considerable importance, they may leave the release of other allergic mediators (e. g., histamine, proteases) unaffected. Such arachidonate pathway inhibitors include inhibitors of 5-lipoxygenase and inhibitors of cyclooxygenase.

As mentioned above, localized administration of several therapeutic agents is appropriate for targeting tissues or organs adversely affected by high levels of renin or angiotensin activity. Hence, the invention contemplates administration of mast cell inhibitors, renin inhibitors, ACE inhibitors, AT₁ receptor antagonists or modulators of the Na⁺/H⁺ exchanger in a localized manner to prevent or treat lung conditions without significantly influencing unaffected tissues and organs.

Bladder Conditions

One aspect of the invention is a method for treating bladder dysfunction and or interstitial cystitis by administering a renin inhibitor or mast cell stabilizer to an animal having bladder dysfunction. The invention is based on the finding that, similar to the vascular tissue, interstitial cystitis is characterized by mast cell hyperplasia in the vicinity of the nerves and smooth muscle layer in the bladder. Bladder smooth muscle expresses angiotensin II receptors which when activated leads to bladder contraction and, according to the invention, release of renin from the mast cells leads to local angiotensin II formation leading to bladder contraction, inflammation, and fibrosis.

Angiotensin II functions as a growth factor that modulates proliferation and hypertrophy of bladder smooth muscle cells and stimulates the production of extracellular matrix proteins. Thus, locally produced angiotensin II is involved in the regulation of cellular and tissue events leading to bladder hypertrophy and remodeling. An overproduction of angiotensin II initiates and maintains bladder hypertrophy and remodeling, while suppressing angiotensin II production, prevents and reverses this process. Therefore, mast cell stabilizers and renin inhibitors can prevent and reverse bladder hypertrophy and remodeling associated with bladder dysfunction.

An effective amount of a mast cell stabilizer or renin inhibitor can be an amount that modifies systemic blood pressure by less than 10% within one day of administration. In one embodiment the cardiac mast cells stabilizer is lodoxamide. The effective amount of the renin-angiotensin system inhibitor can be low enough so that systemic blood pressure is lowered within one day of administration by even less than 5%, and in particular so low as to cause no measurable lowering of systemic blood pressure (i.e., no change in systemic blood pressure acutely).

In one embodiment the renin-angiotensin system inhibitor is administered orally. In another embodiment the renin-angiotensin system inhibitor is administered by a sustained release implant. As mentioned above, localized administration of therapeutic agents is appropriate for targeting tissues or organs adversely affected by high levels of renin or angiotensin activity. Hence, the invention contemplates administration of mast cell inhibitors, renin inhibitors, ACE inhibitors, or AT₁ receptor antagonists in a localized manner to prevent or treat bladder conditions without significantly influencing unaffected tissues and organs.

Inflammatory Bowel Disease

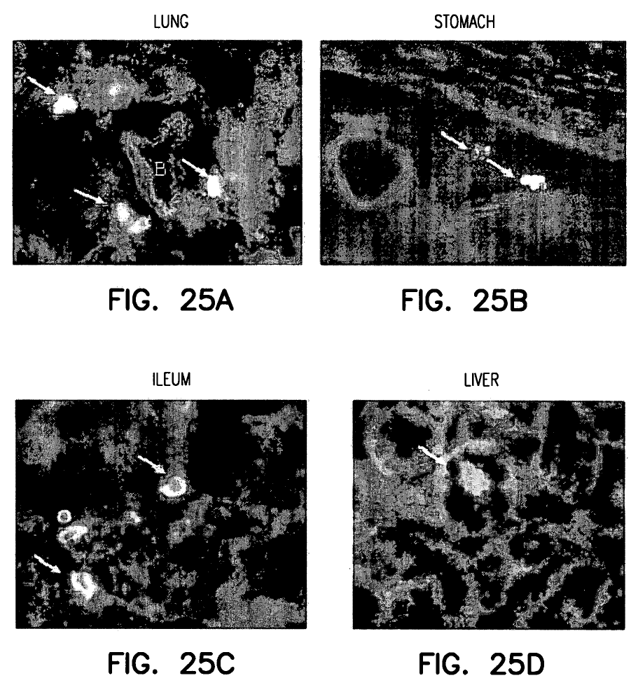

Renin positive mast cells are found in the gut (FIG. 25). It is known that increased numbers of mast cells are observed in the mucosa of the ileum and colon of patients with inflammatory bowel disease. This condition is accompanied by great changes in the content of mast cells such as TNFa, IL-16, substance P, histamine, and tryptase. Evidence of mast cell degranulation has been found in the intestine from patients with inflammatory bowel disease. The mast cell is known to be a key cell type involved in the pathogenesis of inflammatory bowel disease but its role has previously been unclear. According to the invention, renin that is released from mast cells can initiate the local formation of angiotensin II in the mucosa of patients with inflammatory bowel disease, thereby exacerbating the inflammation, and other related pathologies associated with inflammatory bowel disease.

Skin Conditions

In response to a challenge by an allergen, mast cells release renin, and according to the invention renin may also be implicated as a mediator for symptoms of atopic dermatitis and/or infections of wounds or skin lesions. The present invention provides methods for inhibiting renin activity or release from mast cells that involve administering mast cell stabilizers and/or renin inhibitors for the treatment of atopic eczema, infections of wounds or skin lesions.

The prime objectives of treatment are to reduce inflammation and to prevent and relieve itching. Itching leads to scratching and to trauma of the skin, resulting in infection, lichenification, and eczematization. The present invention provides a topical composition comprising an effective amount of a mast cell stabilizer and/or a renin inhibitor that can be used to inhibit the release and/or activity of renin. Mast cell stabilizers include cromolyn sodium, nedocromil and lodoxamide. Such a composition may be used alone or optionally, in combination with one or more added therapeutic agents such as ACE inhibitors, AT₁ receptor antagonists or modulators of the Na⁺/H⁺ exchanger (NHE).

Administration

The peptides, nucleic acids and compounds of the invention, including their salts, are administered so as to achieve a reduction in at least one symptom associated with an indication or disease.

To achieve the desired effect(s), the compounds, nucleic acids and peptides may be administered as single or divided dosages, for example, of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the compound, nucleic acid or peptide chosen, the disease, the weight, the physical condition, the health, the age of the mammal, whether prevention or treatment is to be achieved, and if the peptide is chemically modified. Such factors can be readily determined by the clinician employing animal models or other test systems that are available in the art.

Administration of the therapeutic agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the compounds, nucleic acids or peptides of the invention may be essentially continuous over a preselected period of time or may be in a series of spaced doses. Both local and systemic administration is contemplated, however, in some embodiments, local administration is preferred.

To prepare the composition, compounds, nucleic acids and/or peptides are synthesized or otherwise obtained, purified as necessary or desired and then lyophilized and stabilized. The compound, nucleic acid or peptide can then be adjusted to the appropriate concentration, and optionally combined with other agents. The absolute weight of a given compound, nucleic acid or peptide included in a unit dose can vary widely. For example, about 0.01 to about 2 g, or about 0.1 to about 500 mg, of at least one peptide, nucleic acid or compound of the invention, or a plurality of compounds, nucleic acids and/or peptides specific for a particular mast cell type or renin isotype can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the compounds, nucleic acids and peptides of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

Thus, one or more suitable unit dosage forms comprising the therapeutic peptides, nucleic acids and compounds of the invention can be administered by a variety of routes including oral, parenteral (including subcutaneous, intravenous, intramuscular and intraperitoneal), rectal, dermal, transdermal, intrathoracic, intrapulmonary and intranasal (respiratory) routes. The therapeutic peptides, nucleic acids and compounds may also be formulated for sustained release (for example, using microencapsulation, see WO 94/ 07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system.

When the therapeutic peptides, compounds and/or nucleic acids of the invention are prepared for oral administration, they are generally combined with a pharmaceutically acceptable carrier, diluent or excipient to form a pharmaceutical formulation, or unit dosage form. For oral administration, the peptides may be present as a powder, a granular formulation, a solution, a suspension, an emulsion or in a natural or synthetic polymer or resin for ingestion of the active ingredients from a chewing gum. The peptides, compounds and/or nucleic acids may also be presented as a bolus, electuary or paste. Orally administered therapeutic compounds, nucleic acids and peptides of the invention can also be formulated for sustained release, e.g., these agents can be coated, micro-encapsulated, or otherwise placed within a sustained delivery device. The total active ingredients in such formulations comprise from 0.1 to 99.9% by weight of the formulation.

By “pharmaceutically acceptable” it is meant a carrier, diluent, excipient, and/or salt that is compatible with the other ingredients of the formulation, and not deleterious to the recipient thereof.

Pharmaceutical formulations containing the therapeutic compounds, nucleic acids or peptides of the invention can be prepared by procedures known in the art using well-known and readily available ingredients. For example, a selected compound, nucleic acid, peptide or combination thereof, can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatin, and polyvinyl-pyrrolidone. Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethyl glycols can also be included. Preservatives may also be added. The compositions of the invention can also contain thickening agents such as cellulose and/or cellulose derivatives. They may also contain gums such as xanthan, guar or carbo gum or gum arabic, or alternatively polyethylene glycols, bentones and montmorillonites, and the like.

For example, tablets or caplets containing the compounds, nucleic acids and peptides of the invention can include buffering agents such as calcium carbonate, magnesium oxide and magnesium carbonate. Caplets and tablets can also include inactive ingredients such as cellulose, pre-gelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and the like. Hard or soft gelatin capsules containing at least one compound or peptide of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric-coated caplets or tablets containing one or more peptides, nucleic acids or compounds of the invention are designed to resist disintegration in the stomach and dissolve in the more neutral to alkaline environment of the duodenum.

The therapeutic peptides, nucleic acids and compounds of the invention can also be formulated as elixirs or solutions for convenient oral administration or as solutions appropriate for parenteral administration, for instance by intramuscular, subcutaneous, intraperitoneal or intravenous routes. The pharmaceutical formulations of the invention can also take the form of an aqueous or anhydrous solution or dispersion, or alternatively the form of an emulsion or suspension or salve.

Thus, the therapeutic peptides, nucleic acids or compounds may be formulated for parenteral administration (e.g., by injection, for example, bolus injection or continuous infusion) and may be presented in unit dose form in ampoules, pre-filled syringes, small volume infusion containers or in multi-dose containers. As noted above, preservatives can be added to help maintain the shelf life of the dosage form. The active peptides, nucleic acids and other ingredients may form suspensions, solutions, or emulsions in oily or aqueous vehicles, and may contain formulatory agents such as suspending, stabilizing and/or dispersing agents. Alternatively, the active peptides, compounds, nucleic acids and other ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable carriers, vehicles and adjuvants that are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol,” polyglycols and polyethylene glycols, C₁-C₄ alkyl esters of short-chain acids, ethyl or isopropyl lactate, fatty acid triglycerides such as the products marketed under the name “Miglyol,” isopropyl myristate, animal, mineral and vegetable oils and polysiloxanes.

It is possible to add, if necessary, an adjuvant chosen from antioxidants, surfactants, other preservatives, film-forming, keratolytic or comedolytic agents, perfumes, flavorings and colorings. Antioxidants such as t-butylhydroquinone, butylated hydroxyanisole, butylated hydroxytoluene and ce-tocopherol and its derivatives can be added.

Additionally, the peptides, nucleic acids or compounds are well suited to formulation as sustained release dosage forms and the like. Such sustained release formulations can be administered locally to specific tissues or organs. The formulations can also be constituted so that they release the active peptide, nucleic acid or compound, for example, in a particular part of the intestinal, vascular or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, draining devices and the like.

For topical administration, the therapeutic agents may be formulated as is known in the art for direct application to a target area. Forms chiefly conditioned for topical application take the form, for example, of creams, milks, gels, dispersion or microemulsions, lotions thickened to a greater or lesser extent, impregnated pads, ointments or sticks, aerosol formulations (e.g., sprays or foams), soaps, detergents, lotions or cakes of soap. Other conventional forms for this purpose include wound dressings, coated bandages or other polymer coverings, ointments, creams, lotions, pastes, jellies, sprays, and aerosols. Thus, the therapeutic agents of the invention can be delivered via patches or bandages for dermal administration. Alternatively, the peptide, nucleic acid or compound can be formulated to be part of an adhesive polymer, such as polyacrylate or acrylate/vinyl acetate copolymer. For long-term applications it might be desirable to use microporous and/or breathable backing laminates, so hydration or maceration of the skin can be minimized. The backing layer can be any appropriate thickness that will provide the desired protective and support functions. A suitable thickness will generally be from about 10 to about 200 microns.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active peptides and compounds can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-85% by weight.

Drops, such as eye drops or nose drops, may be formulated with one or more of the therapeutic peptides, nucleic acids or compounds in an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The therapeutic peptides, nucleic acids or compounds may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; and mouthwashes comprising the composition of the present invention in a suitable liquid carrier.

The pharmaceutical formulations of the present invention may include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the present invention include water and physiologically acceptable buffered saline solutions such as phosphate buffered saline solutions pH 7.0-8.0.

The peptides, nucleic acids and compounds of the invention can also be administered to the respiratory tract. Thus, the present invention also provides aerosol pharmaceutical formulations and dosage forms for use in the methods of the invention. In general, such dosage forms comprise an amount of at least one of the agents of the invention effective to treat or prevent the clinical symptoms of a specific infection, indication or disease. Any statistically significant attenuation of one or more symptoms of an infection, indication or disease that has been treated pursuant to the method of the present invention is considered to be a treatment of such infection, indication or disease within the scope of the invention.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatin or blister packs from which the powder may be administered with the aid of an inhalator, insufflator, or a metered-dose inhaler (see, for example, the pressurized metered dose inhaler (MDI) and the dry powder inhaler disclosed in Newman, S. P. in Aerosols and the Lung, Clarke, S. W. and Davia, D. eds., pp. 197-224, Butterworths, London, England, 1984).

Therapeutic peptides, nucleic acids or compounds of the present invention can also be administered in an aqueous solution when administered in an aerosol or inhaled form. Thus, other aerosol pharmaceutical formulations may comprise, for example, a physiologically acceptable buffered saline solution containing between about 0.1 mg/ml and about 100 mg/ml of one or more of the peptides or compounds of the present invention specific for the indication or disease to be treated. Dry aerosol in the form of finely divided solid peptide or nucleic acid, or particles of selected compounds that are not dissolved or suspended in a liquid are also useful in the practice of the present invention. Peptides, nucleic acids and compounds of the present invention may be formulated as dusting powders and comprise finely divided particles having an average particle size of between about 1 and 5 μm, alternatively between 2 and 3 μm. Finely divided particles may be prepared by pulverization and screen filtration using techniques well known in the art. The particles may be administered by inhaling a predetermined quantity of the finely divided material, which can be in the form of a powder. It will be appreciated that the unit content of active ingredient or ingredients contained in an individual aerosol dose of each dosage form need not in itself constitute an effective amount for treating the particular condition, indication or disease since the necessary effective amount can be reached by administration of a plurality of dosage units. Moreover, the effective amount may be achieved using less than the dose in the dosage form, either individually, or in a series of administrations.

For administration to the upper (nasal) or lower respiratory tract by inhalation, the therapeutic agents of the invention are conveniently delivered from a nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Nebulizers include, but are not limited to, those described in U.S. Pat. Nos. 4,624,251; 3,703,173; 3,561,444; and 4,635,627. Aerosol delivery systems of the type disclosed herein are available from numerous commercial sources including Fisons Corporation (Bedford, Mass.), Schering Corp. (Kenilworth, N.J.) and American Pharmoseal Co., (Valencia, Calif.). For intra-nasal administration, the therapeutic agent may also be administered via nose drops, a liquid spray, such as via a plastic bottle atomizer or metered-dose inhaler. Typical of atomizers are the Mistometer (Wintrop) and the Medihaler (Riker).

Furthermore, the active ingredients may also be used in combination with other therapeutic agents, for example, pain relievers, anti-inflammatory agents, antihistamines, antimicrobials, bronchodilators and the like, whether for the conditions described or some other condition.

The present invention further pertains to a packaged pharmaceutical composition for controlling the symptoms of a particular condition or disease such as a kit or other container. The kit or container holds a therapeutically effective amount of a pharmaceutical composition for controlling the selected condition and instructions for using the pharmaceutical composition for control of the condition. The pharmaceutical composition includes at least one compound, nucleic acid or peptide of the present invention, in a therapeutically effective amount such that condition is controlled.

The invention is further illustrated by the following non-limiting Examples.

### EXAMPLE 1

### Materials and Methods

This Example provides materials and methods for many of the procedures employed in the subsequent Examples.

Tissue preparation: Pathogen-free Sprague-Dawley rats of both sexes (Charles River Laboratories, Kingston, N.Y.), weighing between 150 and 300 g and hearts from mast-cell-deficient WBB6F1-W/Wv (Jackson laboratory, stock #100410) and congenic control (WBB6F1-+/+)(CC) rats were used for these experiments. Rats were sacrificed according to approved IACUC guidelines. Briefly, rats were anesthetized with CO₂ vapor, exsanguinated and the hearts were rapidly excised and mounted via aortic cannulation in a Langendorff apparatus. The hearts were washed for 20 min with Krebs-Henseleit buffer to remove blood and fixed by perfuision with 4% paraformaldehyde (PF) at pH 7.4. After 10 min perfuision, the hearts were detached from the apparatus and immersed in 4% PF for an additional hour before rinsing and storing in 30% sucrose for 3 hours to cryoprotect. Kidney, heart, bronchus, ileum, stomach and liver were removed, washed free of blood, fixed, and cryoprotected as above. These tissues were then embedded in tissue freezing medium (Electron Microscopy Sciences) and snap-frozen in liquid nitrogen. Using a Bright cryostat (model OTF), frozen sections of 10 μm were prepared, collected onto clean Fischer Superfrost Plus slides, and stored at −80° C. until ready for immunohistochemistry.

Immuno- and histochemical staining: Native tissues (kidney, heart, bronchus, ileum, stomach, liver) were prepared for immunohistochemical or histochemical staining as follows. Slides containing frozen tissue sections were washed for 5 min in PBS. The sections were then permeabilized for 10 min at 37° C. with a solution containing 4% fetal bovine serum (FBS) and 0.3% Triton X-100 dissolved in PBS. After washing the sections with PBS, 10% FBS was applied to the sections for 1 h at 37° C. to block non-specific binding before adding antibodies. After this, primary (1°) antibody (Ab) was applied to the sections for 2 h at 37° C., followed by three washes in PBS. Next, sections were exposed to secondary Ab (all purchased from Molecular Probes) for 1 h at 37° C. Sections were then washed as described above, followed by fixation for 3 min with 4% PF. After washing with PBS, sections were mounted in Vectashield anti-fading solution (Vector Laboratories, Burlingame, Calif.). The procedure of Kiernan et al. (Histological and histochemical methods: Theory and practice. Oxford, Pergamon Press. 1981, 162-163) was followed in experiments where tissue was stained with toluidine blue (Sigma) (0.25% in acetic acid, pH 2.0). In instances where tissues were co-stained, staining with toluidine blue was performed after the tissue had been immunostained.

Primary polyclonal rabbit anti-renin Ab against human recombinant renin (Campbell et al., Hypertension 27: 1121-1133, 1996) was applied to rat kidney, heart sections, sections of bronchus, trachea, ileum, and colon, at a dilution of 1:500. The corresponding secondary Ab used was Alexa fluor 488 donkey anti-rabbit IgG (green) diluted 1:300 or 1:500 for kidney or heart, respectively. For competition experiments, an excess of human renin (Calbiochem) was combined with the polyclonal rabbit anti-renin Ab overnight, before proceeding with immunostaining.

Both a monoclonal mouse anti-renin Ab against rat renin (Swant Scientific, Switzerland) and a polyclonal rabbit anti-histamine Ab (Accurate Chem. Sci. Corp.) were applied to rat heart sections at a dilution of 1:100 and 1:500, respectively. For these sections, the secondary Abs used were a 1:500 dilution of both Alexa fluor 594 goat anti-mouse IgG (red) and Alexa fluor 488 donkey anti-rabbit IgG (green).

Heart sections that were co-labeled with the rabbit anti-renin Ab (1:500) and mouse anti-cathepsin-D Ab (1:500) (Oncogene Research Bioproducts), were subsequently stained using the following secondary Abs: Alexa fluor 594 donkey anti-rabbit IgG (red) (1:300) and Alexa fluor 488 goat anti-mouse IgG (green) (1:300).

A goat anti-synapsin Ia/b Ab (Santa Cruz) was employed for heart sections—this antibody has been shown to be specific for neurons. Sudhof et al., Science 245: 1474-1480, 1989. This Ia/b Ab was applied to heart sections at a dilution of 1:300, and the polyclonal rabbit anti-renin Ab was used at a dilution of 1:500. The following secondary Abs were utilized: Alexa fluor 488 goat anti-mouse IgG (green) (1:300) and Alexa fluor 594 donkey anti-rabbit IgG (red) (1:500).

The human mastocytoma cell line, HMC-1, was provided by Drs. I. Biaggioni and J. H. Butterfield. Cells were maintained in suspension culture at high density in Iscove's modified Dulbecco's medium supplemented with 10% FBS and kept at 37° C., 5% CO₂. For immunocytochemistry, HMC-1 cells were grown on standard 22 mm glass coverslips for 48 h and rinsed free of media with PBS. Cells were then fixed and permeabilized in PBS containing 3.7% PF and 0.3% Triton X-100. Cells were washed with PBS for 3 min and then incubated for 30 min at 37° C. with 1% BSA to block non-specific binding. After this, the cells were incubated with polyclonal rabbit anti-renin Ab (1:400) for 1 h at room temperature. The cells were washed three times for 5 min each with PBS, and then exposed to Alexa fluor 488 donkey anti-rabbit IgG (green) (1:400) for 1 h at room temperature. After washing with PBS as above, the cover slip was mounted onto a microscope slide with Vectashield.

Tissue sections or cells were examined either with an inverted epifluorescent microscope (Nikon Diaphot) interfaced to a frame-transfer type cooled CCD (Roper Scientific) and processed with Metafluor/Metamorph software (Universal Imaging, Inc.) or with a Leica TCS SP2 confocal microscope. Digital images were imported into Adobe Photoshop (5.0) for minimal processing.

Renin activity: Pooled confluent flasks of HMC-1 cells, or individual wells of HMC-1 cells, were pelleted and resuspended in HEPES buffer (pH 5.7, 1 mM EDTA) containing the mast-cell degranulating agent compound 48/80 (100 μg/ml or 20 μg/ml; Sigma). After 30 min, cells were spun down and the renin-containing supernatant was incubated with increasing concentrations of human angiotensinogen (Sigma). Renin activity (ANG I formed) was then determined using a GammaCoat Plasma Renin Activity ¹²⁵I Radioimmunoassay kit (DiaSorin, Mass.). The selective renin inhibitor BILA2157 was from Boehringer Ingelheim.

RT-PCR: Total RNA was extracted from human kidney tissue and HMC-1 cells using RNA STAT-60 reagent (Tel-Test “B” Inc., TX). 1 μg of total RNA from each sample was reverse-transcribed and assayed by RT-PCR using QIAGEN's Onestep RT-PCR Kit (Valencia, Calif.). Sense and anti-sense primers specific for human renin at exons 4 and 7 of the renin gene were 5′-TCTCAGCCAGGACATCATCA-3′ (SEQ ID NO:62) and 5′-AGTGGAAATTCCCTTCGTAA-3′ (SEQ ID NO:63), respectively. Becker et al., Transplantation 69: 1485-1491, 2000. Use of these primers avoided co-amplification of genomic DNA coding for renin. Sense and anti-sense primers employed for a 62 -actin control were 5′ -GCTCGTCGTCGACAACGGCTC-3′ (SEQ ID NO:64) and 5′-GCTCTTCTACTGGGTCTAGTACAAAC-3′ (SEQ ID NO:65). The amplification procedure utilized was: 50° C. 30 min, 95° C. 15 min, then 94° C. 30 sec, 55° C. 30 sec, 72° C. 1 min (40 cycles) and finally 72° C. 10 min. PCR products generated were about 288 bp and about 350 bp, for renin and β-actin respectively. PCR products were analyzed by agarose gel electrophoresis and ethidium bromide staining. The renin RT-PCR product from HMC-1 RNA was extracted from the agarose gel using GENECLEAN II (QBIOgene, Inc. Calif.) and precipitated in ethanol to further purify and concentrate the DNA. Sequencing of the RT-PCR samples was performed by the Rockefeller University's DNA Sequencing Resource Center and run on a SpectruMedix 9610 DNA sequencer.

Western blotting: Samples of rat kidney homogenate (20 μg/lane), HMC-1 lysate (50 μg/lane) and cathepsin D (CD) (500 μg/lane; Sigma) were prepared with 2× Novex Tris-glycine SDS sample buffer (Invitrogen) and boiled for 5 min, before separation on 12% Tris-glycine SDS-polyacrylamide minigels (Invitrogen). Electrophoresis was carried out at 200 V, 40 mA/gel for 1 hr. Gels were soaked in transfer buffer (25 mM Tris-base, 0.2 M glycine, and 10% methanol, pH 8.5) and electrotransferred to polyvinylidene difluoride (PVDF) membranes (Immobilon-P, Millipore, Mass.) for 90 min at 25 V, 100 mA, room temperature. Membranes were blocked for at least 2 hours in blocking buffer (Tris-buffered saline (TBS), containing 0.1% Tween 20, 5% (w/v) non-fat dry milk). Primary antibodies were incubated with the PVDF overnight at 4° C., diluted appropriately in primary Ab dilution buffer (TBS containing 0.1% Tween 20, 5% BSA). The PVDF was washed three times with TBS. Horseradish peroxidase-coupled 2° Ab was then added at a 1:2000 dilution in blocking buffer for 1 hour. After three further TBS washes, the protein of interest was detected using enhanced chemiluminescence (LumiGLO; Cell Signaling Technology Inc.) and by exposure to X-ray film (Biomax MR, Kodak, N.Y.).

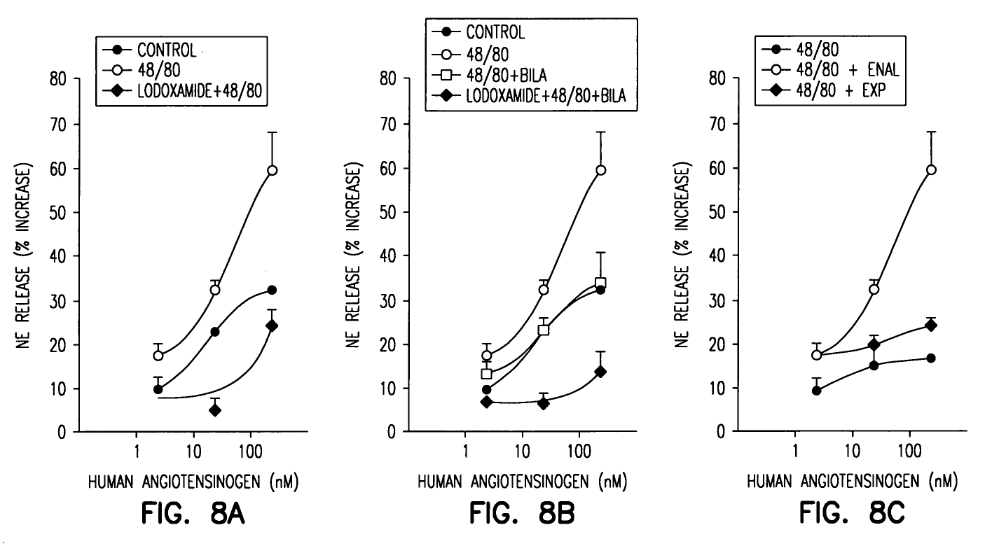

Preparation of guinea-pig sympathetic nerve endings: Cardiac sympathetic nerve endings were isolated from guinea-pig hearts as previously described (Seyedi et al., J Pharmacol Exp Ther 290: 656-663, 1999; Seyedi et al., Circ. Res. 81: 774-784, 1997). This procedure was utilized to isolate the sympathetic nerve endings for generating the data shown in FIG. 8. Spontaneously beating hearts were isolated as previously described by the inventors (Hatta et al., J. Pharmacol. Exp. Ther. 288: 919-927, 1999; Park et al., Circ. Res. 71: 992-1001, 1992). Briefly, male Hartley guinea pigs were anesthetized with CO₂ vapor and then exsanguinated. Hearts were perfused through the aorta for 15 min at constant pressure with Ringer's solution at 37° C. (Park et al., Circ. Res. 71: 992-1001, 1992) to exclude blood from the coronary circulation. Hearts were minced in 0.32 M sucrose containing 1 mM EGTA. The minced tissue was then digested with 75 mg collagenase per 10 ml HBS per gm for 1 hr at 37° C. After low speed centrifugation the resulting pellet were suspended in 10 volumes of 0.32 M sucrose and homogenized with a Teflon/glass homogenizer. The homogenate was centrifuged at 650 g for 10 min, the pellet was re-homogenized and the homogenate was centrifuged again. The pellet containing cellular debris was discarded, the supernatants from the last two spins were combined, the pooled supernatant was equally subdivided into 4 to 8 tubes and re-centrifuged for 20 min at 20,000 g at 4° C. This pellet contains cardiac sympathetic nerve endings. The pellet was resuspended either in HEPES-buffered saline (HBS; 500 μl, normoxic conditions) or in glucose-free HBS, which contained the reducing agent sodium dithionite (500 μl, ischemic conditions), and incubated in the absence or presence of angiotensinogen (1 hr) or other agents for 20 min at 37° C. prior to ischemia (see below). Each suspension was used only once as an independent sample.

Ischemia-reperfusion in the mouse heart ex vivo: KO and CC mice will be anesthetized with CO₂ vapor and killed by exsanguination (IACUC approved). The heart will be rapidly excised and transferred to a Langendorff apparatus. See, Koyama et al., Mol. Pharmacol 63: 378-382 (2003). The aorta will be cannulated with a flanged 20-gauge stainless-steel needle. Surface ECG will be obtained from leads attached to the apex of the left ventricle and the aortic cannula, and recorded in both analog and digital format. See Koyama et al., Biochemical and Biophysical Research Communications 306(3):792-796 (2003). Spontaneously beating hearts will be perfused through the aorta at a constant pressure of 100 cm H₂O with Krebs-Henseleit buffer (KHB). After stabilization, normothermic global ischemia will be induced by complete cessation of coronary perfusion, followed by 45-min reperfusion. The coronary effluent is analyzed for renin activity and NE.

Ischemia-reperfusion in guinea pig hearts ex vivo: Guinea-pig hearts will be perfused for 30 min prior to ischemia. Imamura et al., Circ. Res. 78: 475-481 (1996). Normothermic 20-min global ischemia will be induced by complete cessation of coronary perfusion, followed by 45-min reperfusion. Coronary effluent will be assayed for renin activity and NE.

Norepinephrine assay: Coronary effluent and synaptosome supernatant will be assayed for NE by HPLC with electrochemical detection (ESA). Hatta et al., Circulation 96, 1-498 (1997); Sudhof et al., Science 245: 1474-1480 (1989); Silver et al., Proc. Natl. Acad. Sci. USA 99: 501-506 (2002). The detection limit is about 0.2 pmol.

Degranulation and Renin siRNA Transfection: HMC-1 cells were grown for one day in 6-well culture plates. Cells were counted and degranulated with 48/80 (20 μg/ml). The initial cell releasate was collected, followed by transfection of the cells with either renin specific siRNA (50 nM), or for control, scrambled siRNA (50 nM) (Dharmacon, CO). Renin-specific duplexes were based upon the coding sequence of human renin exon 2-8 (accession #M26900). Cells were maintained in the presence of transfection reagents for 48 hours. After which, cells were counted, degranulated, and the releasate measured by ANG I RIA.

Isolated Bronchial Rings—Tissue preparation. Pathogen-free Male Sprague-Dawley rats (Charles River Breeding Laboratories), weighing between 150 and 300 g, were used for these experiments. Rats were killed according to approved International Animal Care and Use Committee guidelines. Briefly, rats were anesthetized with CO₂ vapor and exsanguinated, and the hearts and lungs were rapidly removed en bloc and placed in normal physiological saline (PSS) or Krebs-Henseleit (KH) buffer. Trachea and bronchial tree were carefully dissected free of parenchymal lung tissue, connective tissue and fat using a dissecting microscope, the blood clots on the smooth muscle surface removed by gentle rubbing with a cotton-tipped applicator stick, and the bronchia and tracheal preparations cut into small rings (3-4 mm length×3-4 mm internal diameter). Preparations were used immediately for experiments.

Tissue bath experiments: All preparations were suspended on tissue hooks in 20 ml organ baths (Radnoti 4-Unit Tissue Organ Bath System) containing Krebs-Henseleit (KH) solution (composition, mM: NaCl: 118.2; KCl: 4.83; NaHCO₃: 2.5; CaCl₂: 2.37; MgSO₄: 2.5; KH₂PO₄: 1.0; EDTA: 0.05 and dextrose: 11.0) gassed with 5% CO₂ in 02 at 37° C. (pH 7.4). Resting tensions were kept at 200 mg in rat bronchus (RB). Mechanical responses were recorded isometrically via the Radnoti force transducers connected to a PowerLab/8SP data acquisition system and recorded and analyzed the data with Chart5 for Windows. The bath fluid was initially changed at 15 min intervals during a 60-90 min equilibration period. In the rat bronchus, high K⁺ (80 mM KCI) was first added and after the plateau of the contraction, washed with fresh normal KH solution. Repeat the procedure once or twice until get a maximal constant contraction was induced by KCI (80 mM) for reference values. Drugs were administered 30 min after the KCl cycle.

At the end of the experimental protocol, a maximal contraction was evoked by addition of KCl (80 mM) in the rat bronchus. After the experiments, the wet weights of the preparations were determined after blotting on a filter paper.

### EXAMPLE 2

### Renin Protein Exists in Mast Cells

Detection of renin was performed using site-specific renin antibodies. The polyclonal anti-renin antibody, BR1, had been raised in rabbit against human recombinant renin as described in Campbell et al., Hypertension 27: 1121-1133, 1996. This BR1 antibody was also tested in rat kidney to assess the specificity of the antibody.

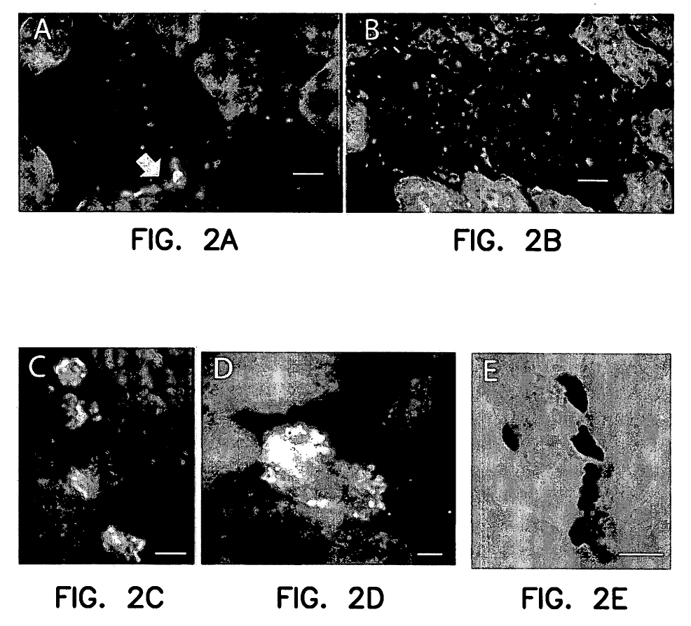

FIG. 2 shows that the anti-renin antibody recognizes renin in the kidney, the traditional source of renin (panel A) in the body. FIG. 2A shows that the vascular pole of the glomerulus, the site of renin synthesis in the kidney, immunoreacted with the anti-renin antibody (see arrow). As a negative control, rat kidney sections were treated with anti-renin antibody pre-adsorbed with an excess of human renin. As shown in FIG. 2B, there was no immunostaining under this condition.

FIG. 2 also illustrates the invention whereby renin protein is recognized by anti-renin antibody in mast cells found in native tissue, as in this example, heart tissue (FIG. 2C and 2D). In the heart sections, the granulated cells were the only cells in the section to be stained with the anti-renin antibody. No other cell type in the ventricle, such as myocytes and nerves, immunoreacted with the antibody.

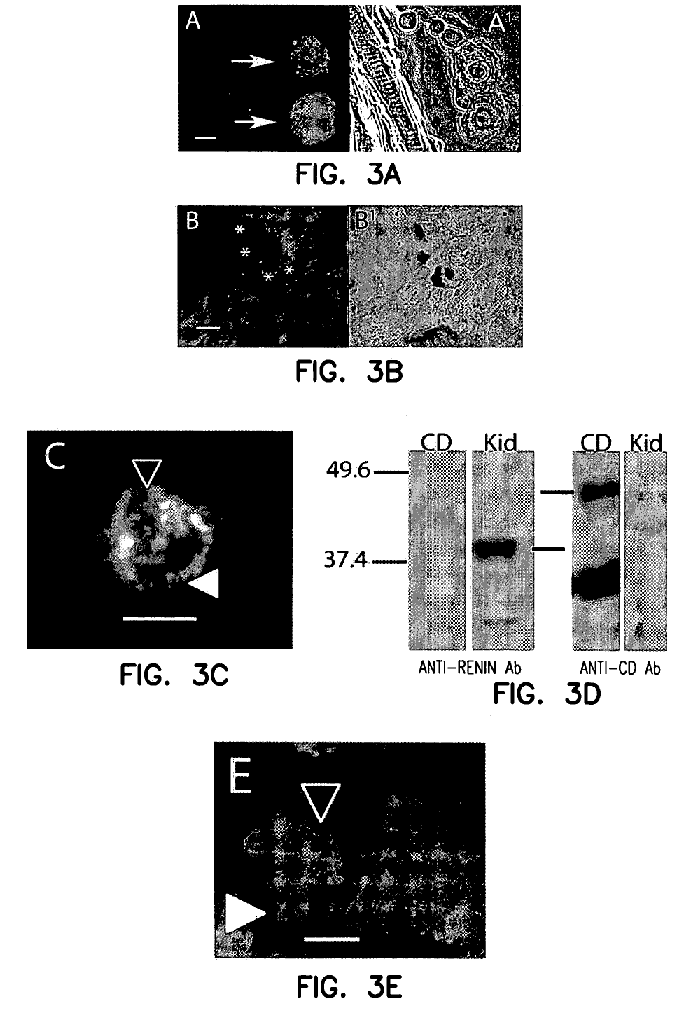

FIG. 3 proves that the cell type immuno-positive for anti-renin antibody in this example of rat ventricle is the mast cell. This figure demonstrates that renin-positive cells also stain for toluidine blue, a classical histochemical stain for mast cells (FIG. 3A and 3A¹). FIG. 3E is a typical transmitted light image showing toluidine-blue staining in a fixed section. Every section analyzed contained toluidine-blue positive cells. To determine if the cells stained with the anti-renin antibody were also toluidine-blue positive, sections were co-stained with both reagents. FIG. 3A and 3A¹ is a single section of rat ventricle viewed with epifluorescence and transmitted light. The arrows point to two cells that immunoreacted with the anti-renin antibody (FIG. 3A) (green). These same cells were also stained with toluidine blue (FIG. 3A¹), thereby illustrating that the anti-renin antibody reacts with renin in cardiac mast cells. The specificity of the anti-renin Ab for mast-cell renin was also determined in sections of heart (FIG. 3B and FIG. 3B¹). A section of ventricle was treated with the anti-renin antibody pre-adsorbed with excess human renin (FIG. 3B) and then stained with toluidine blue, as a means of identifying mast cells (FIG. 3B¹). This section contained toluidine-blue-positive mast cells, as shown in the transmitted light image. Their corresponding position is indicated by the asterisks in the fluorescence image; clearly, these cells did not immunoreact with the pre-adsorbed anti-renin antibody. These results are further proof that the anti-renin antibody is recognizing renin in cardiac mast cells.

Because mast cells are known to contain histamine, heart sections were stained with an anti-histamine antibody as a definitive measure for classifying the sub-population of cells that stained with the anti-renin antibody as mast cells (FIG. 3C). Sections of rat heart were co-stained with the polyclonal rabbit anti-histamine antibody (green, open arrowhead) and the monoclonal mouse anti-renin antibody (red, closed arrowhead). Overlapping areas of staining appeared yellow. The histamine-containing mast cells stained with both antibodies, as shown in FIG. 3C, corroborating the earlier identification of these renin-containing cells as mast cells. No other staining was observed.

FIG. 3D also demonstrates that the anti-renin antibody was not cross-reacting with other proteases found in mast cells. Mast cells contain the protease cathepsin D, which is present in endosomal and lysosomal compartments of mast cells, and capable of cleaving angiotensinogen to ANG I, via a renin-independent pathway. BLAST analysis reveals that renin and cathepsin D are 60% homologous at the amino acid level. Western blots of kidney homogenate and pure cathepsin D that were probed with the polyclonal anti-renin antibody showed that kidney homogenate displayed bands consistent with the presence of renin at ˜42 kD, whereas there was no reactivity to the anti-renin antibody in the lane loaded with pure cathepsin D. The other half of the gel, also loaded with rat kidney homogenate and cathepsin D, was probed with an anti-cathepsin D antibody. The lane loaded with cathepsin D showed distinct bands typical of cathepsin D at 48 kD and 34 kD, the lower band corresponding to a subunit of cathepsin D. The kidney homogenate displayed a faint band at 48 kD, consistent with the known paucity of cathepsin D in the kidney. Importantly, there was no band associated with renin in the homogenate. These results demonstrate that the anti-renin and anti-cathepsin D antibodies do not cross react with cathepsin D and renin, respectively.

In addition, sections of rat heart were co-stained with both anti-renin and anti-cathepsin D antibodies to determine if renin and cathepsin D could be co-localized to the same cells (FIG. 3E). Sections of frozen rat ventricle were exposed to the polyclonal anti-renin antibody (red, closed arrowhead) and cathepsin D antibody (green, open arrowhead). Mast cells in the heart stained with both antibodies, and by two-dimensional analysis, did so in separate compartments (FIG. 3E). No other cells stained with both antibodies.

### EXAMPLE 3

### Mast Cells Express and Synthesize Active Renin

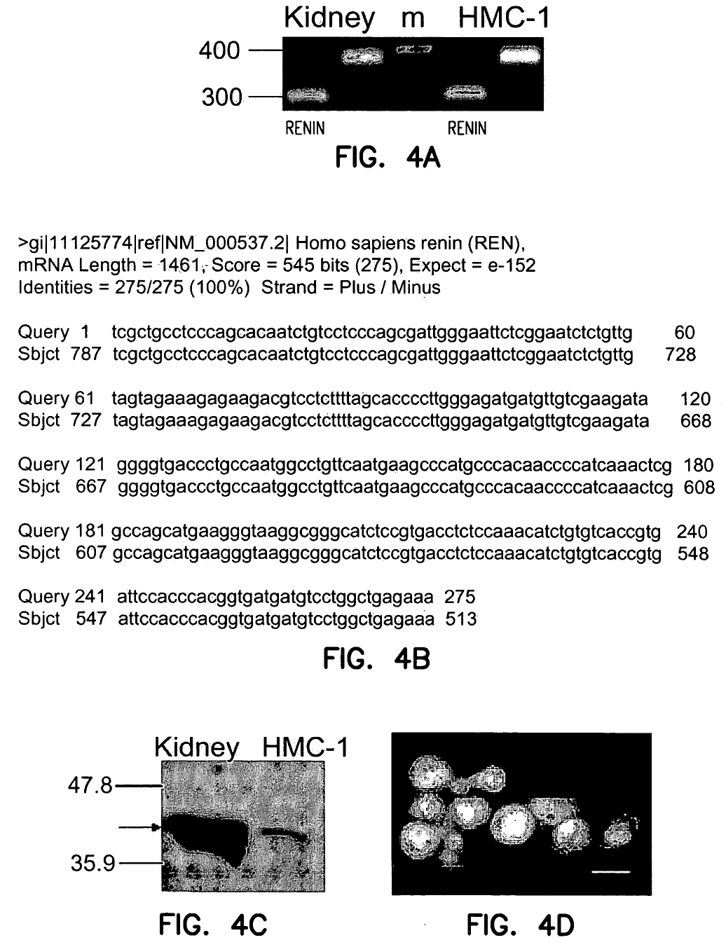

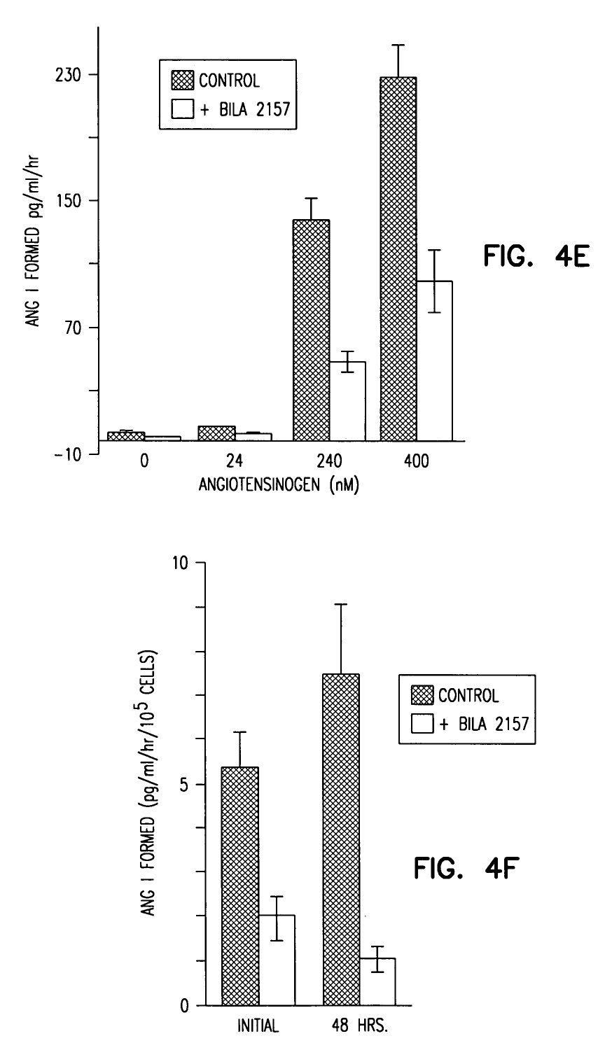

FIG. 4 demonstrates that mast cell-derived renin is active when released from mast cells and is capable of cleaving angiotensinogen which leads to ANG I formation. To demonstrate this, the chymase-deficient human mast-cell line, HMC-1 (Nilsson et al., Scand. J Immunol 39: 489-498, 1994) was utilized. Because the presence of renin in mast cells had never been reported, before attempting to measure renin activity, we determined whether HMC-1 express renin mRNA and renin protein. Total RNA (1 μg) was extracted from HMC-1, reversed-transcribed, and assayed by PCR using sense and anti-sense primers specific for human renin at exons 4 and 7 of the renin gene to avoid co-amplification of genomic DNA coding for renin.

FIG. 4A is an ethidium-bromide-stained gel showing that the HMC-1 renin PCR product (lane 4) is similar to renin product from human kidney (lane 1). To further characterize the RT-PCR product from HMC-1, the DNA band was extracted from the gel and sequenced at the Rockefeller Univ. Sequencing Facility. The reported nucleotide sequence was then compared to the known sequence of Homo sapiens renin mRNA by BLAST analysis. There was 100% sequence identity between the sequence from HMC-1 and human renin (FIG. 4B), further establishing that renin is expressed in mast cells. HMC-1 cells also express renin protein as shown by Western blot analysis (FIG. 4C). Western analysis of HMC-1 homogenate, probed with the anti-renin antibody, showed a ˜42 kD band for renin, similar to that seen in the kidney. The HMC-1 cells also displayed immunoreactivity to the anti-renin antibody (FIG. 4D).

FIG. 4E shows that renin that is released from mast cells is active i.e. it cleaves angiotensinogen to form ANG I. Cells were grown to confluence and degranulated with the mast-cell-degranulating compound 48/80 {Levi, 1980 #9233}. The cell releasate was incubated with increasing concentrations of angiotensinogen and then assayed for ANG I formed (i.e. renin activity) by RIA. A concentration-response curve for ANG I formation as a function of angiotensinogen concentration was generated on the HMC-1 releasate. The amount of ANG I formed increased with angiotensinogen concentration. To account for a possible contribution to ANG I formation by cathepsin D, which is also capable of cleaving angiotensinogen, though at a rate 10⁵ times slower than renin (Hackenthal, et al. Biochim. Biophys. Acta 522: 574-588 (1978)). ANG I was generated either in the absence or presence of the specific renin inhibitor, BILA2157 (100 nM; IC₅₀ =1 nM) (Simoneau et al., Bioorg. Med. Chem. 7: 489-508 (1999)). As shown in FIG. 4E, ˜70% of the total ANG I formed originates from renin. These results demonstrate that mast-cell-derived renin is active.

FIG. 4F illustrates that mast-cell-derived renin can be synthesized in mast cells. Releasates were analyzed from the same population of HMC-1 exposed to compound 48/80 (20 μg/ml) 48 hours apart, and analyzed for BILA2157-sensitive renin activity. As shown in FIG. 4F, about 70% of the ANG I activity measured in the presence of 240 nM angiotensinogen, is due to BILA2157-sensitive renin, both initially and 48 hours after degranulation with 48/80. These results indicate that HMC-1 can re-synthesize renin.

### EXAMPLE 4

### siRNA Can Inhibit Synthesis of Renin Protein in Mast Cells

FIG. 5A shows that post-transcriptional gene silencing technology inhibited sequence-specific mRNA for renin in the HMC-1 cells. Gene-specific small interfering RNA (siRNA) with the following sequences were used:

| | |
|---|---|
| GAGAAAGGCTGGACAGAGA | (SEQ ID NO:58) |
| TCAACTGGCTGGCCTCTTA | (SEQ ID NO:59) |
| GTACAGCACTTTTCTATTT | (SEQ ID NO:60) |
| GCAAAGAGAGTACATAACA | (SEQ ID NO:61) |

Renin activity or ANG I formed was determined on releasates from a defined population of HMC-1 cells before and 48 hrs. post-transfection. The effect of the siRNA on the amount of renin protein produced by the cells was determined by comparing the initial and final amounts of ANG I formed by the releasates (FIG. 5). In the HMC-1 transfected with siRNA, there was an approximate 40% decrease in the absolute amount of ANG I formed compared to the amount measured from these cells before transfection. There was no significant difference in the amount of ANG I formed either in HMC-1 exposed to scrambled RNA or in cells only exposed to the transfection medium (gene silencer). These results demonstrate that renin synthesis can be interrupted in mast cells.

FIG. 5B shows immunostained control HMC-1 cells and HMC-1 cells transfected with siRNA specific for renin and immunostained with the polyclonal anti-renin antibody (1:400). The HMC-1 cells transfected with siRNA displayed much reduced immunofluorescence compared to control, indicating that synthesis of renin protein was considerably less in the cells exposed to the siRNA.

### EXAMPLE 5

### Mast-cell Degranulation Leads to Renin Release and Exocytotic Norepinephrine Release

Mast cells degranulate in response to myocardial ischemia/reperfusion. Moreover, exogenous ANG II can enhance norepinephrine release from cardiac nerve endings. This Example illustrates the spatial relationship between cardiac mast cells, which according to the invention can release renin, and nerves in heart.

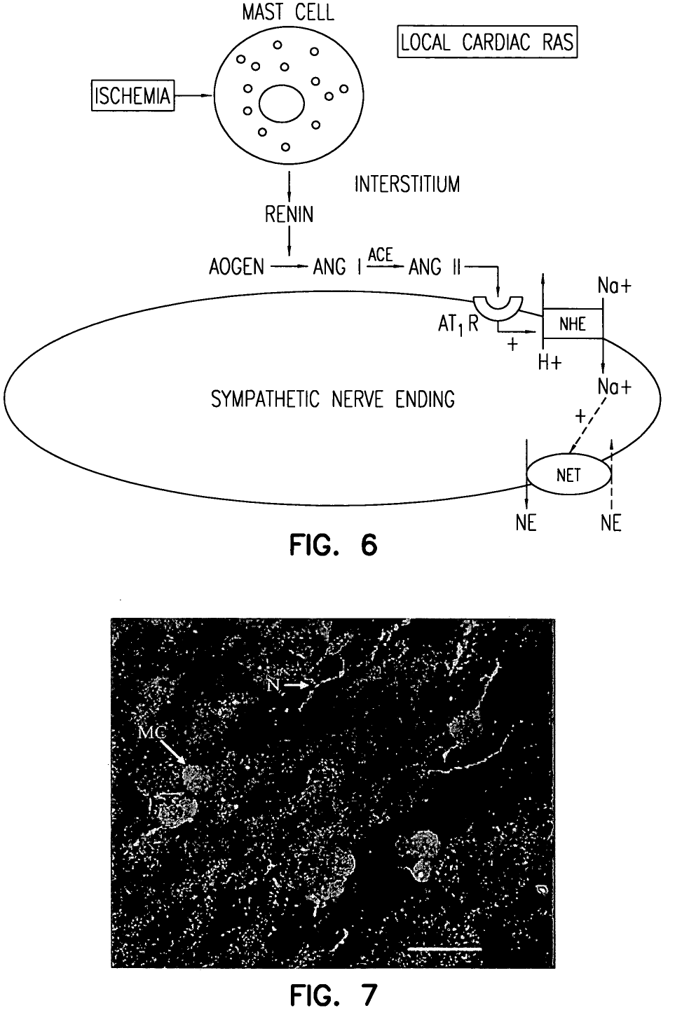

FIG. 7 is a section of rat ventricle stained both with the anti-renin Ab (red) and anti-synapsin I1/b Ab (green) viewed with a confocal microscope. This representative section demonstrates that mast cells (MC) are closely apposed to the nerves (N) in heart. According to the present invention and as outlined in FIG. 6, mast cell degranulation that occurs with myocardial ischemia/reperfusion, is a pivotal event in local RAS activation, initiation of ANG II formation, and the stimulation of ANG II receptors expressed on nerve endings, causing excessive release of norepinephrine leading to arrhythmias. The close proximity of mast cells to nerves supports the claim that ANG II produced from mast-cell derived renin can act on ANG II receptors located on the nerves.

The hypothesis that renin is secreted and operates locally also supported by the data presented in FIG. 8 from cardiac sympathetic nerve endings. Isolated sympathetic nerve endings were isolated from control guinea-pig hearts and incubated with angiotensinogen. As shown in FIG. 8A, norepinephrine release from sympathetic nerve endings increased with the concentration of angiotensinogen in the incubation medium. Furthermore, norepinephrine release was potentiated by pre-treatment of the hearts with 48/80, and markedly attenuated when hearts were perfused with the mast-cell stabilizer lodoxamide prior to 48/80. Importantly, the potentiating effect of 48/80 was abolished either by the selective renin inhibitor BILA2157 or by the ACE inhibitor enalaprilat (Kᵢ, 0.1 nM) (144) or the ANG II receptor (AT₁R) antagonist EXP3174 (Kᵢ, 10 nM) (FIGS. 8B and 8C). According to the invention, these data demonstrate that renin derived from cardiac mast cells is capable of driving ANG II formation leading to potentiation of norepinephrine release from cardiac nerves.

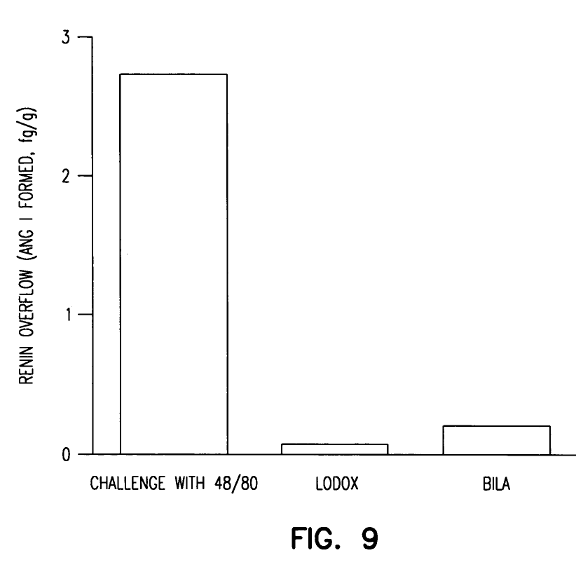

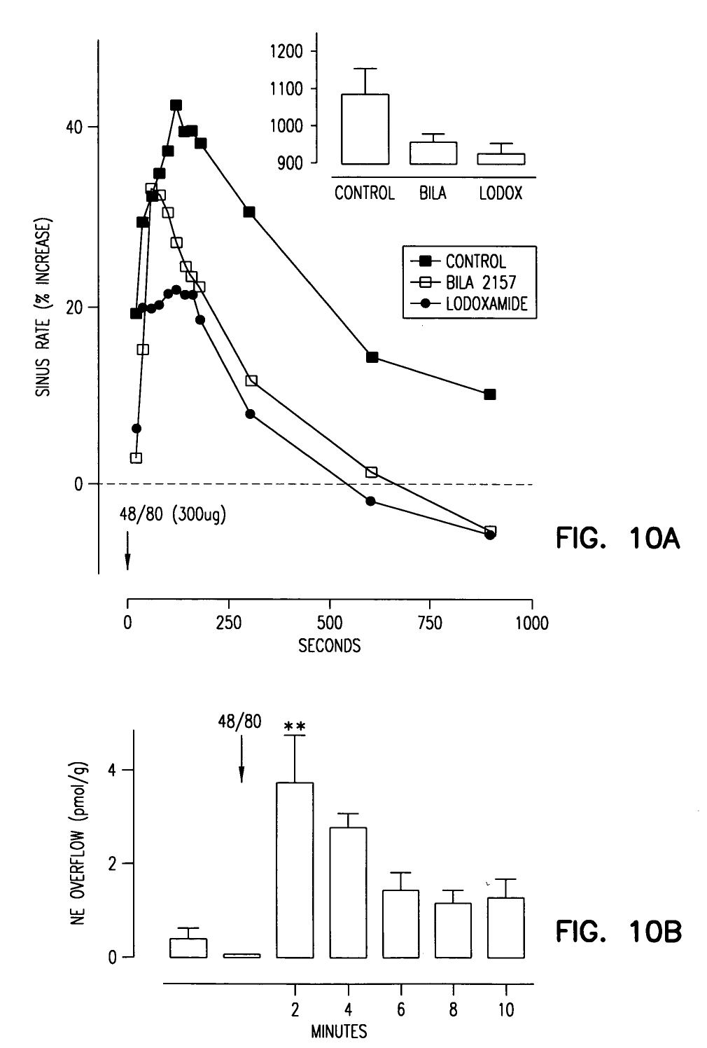

Renin was measured in the coronary effluent of normoxic Langendorff-perfused guinea-pig hearts, ex vivo. FIG.9A shows that there was no measurable renin in the coronary effluent of Langendorff-perfused guinea-pig hearts at the end of the initial 30-minute equilibration period. However, following challenge with the mast cell degranulating agent, 48/80, active renin was released into the coronary effluent. When hearts were perfused with the mast-cell stabilizer lodoxamide (FIG. 9B) or the renin inhibitor BILA2157 prior to 48/80 challenge (FIG. 9 A and B), renin overflow was abolished.

FIG. 10 shows that challenge with 48/80 elicited an increase in sinoatrial rate in these normoxic hearts; this chronotropic response was markedly abbreviated by lodoxamide or BILA2157 (left panel) indicating a relationship between renin release and sinus node stimulation. In addition, the administration of 48/80 elicited norepinephrine overflow in these normoxic hearts (right panel). These findings indicate that cardiac mast cells are capable of releasing active renin, which then leads to local formation of ANG II causing norepinephrine exocytosis.

### EXAMPLE 6

### Mast-cell Degranulation Leads to Renin Release and Ventricular Fibrillation During Myocardial Ischemia

FIG. 11 shows that ischemia causes release of renin from cardiac mast cells, initiating a cascade of events that culminate in reperfusion arrhythmias. Guinea-pig hearts were perfused ex vivo at constant pressure in a Langendorff apparatus and subjected to 20-min. stop-flow global ischemia, followed by 45-min. reperfusion (Hatta, E., R. Maruyama, S. J. Marshall, M. Imamura, and R. Levi. Bradykinin promotes ischemic norepinephrine release in guinea pig and human hearts. J. Pharmacol. Exp. Ther. 288: 919-927 (1999)). Surface ECG and coronary flow were continuously monitored; the coronary effluent was assayed for renin activity (i.e., ANG I generated as described herein). There was no detectable renin in the coronary effluent prior to ischemia. With reperfusion, there was significant renin overflow into the coronary effluent accompanied by ventricular fibrillation (VF) (FIG. 12).

FIG. 12 shows representative ECG tracings from three spontaneously beating guinea-pig hearts perfused in a Langendorff apparatus and subjected to 20-min global ischemia followed by 45-min reperfusion. Traces in the left panels were recorded at the end of the equilibration period prior to ischemia, showing normal sinus rhythm. Traces in the right panels were recorded during reperfusion, showing ventricular fibrillation in the untreated heart (C) and no ventricular fibrillation in the BILA2157- (BILA, 100 nM) and lodoxamide-treated (LODOX, 10 μM) hearts.

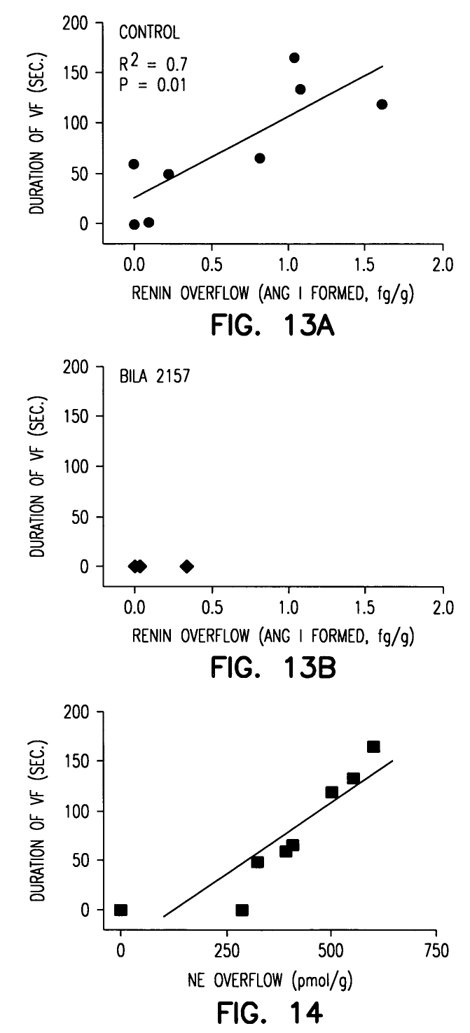

FIG. 13 shows the correlation between renin release and severity of arrhythmias experimentally observed in that the duration of ventricular fibrillation increased with the magnitude of renin overflow. When hearts were treated with the renin inhibitor BILA2157, and then subjected to ischemia and reperfusion, the overflow of active renin was abrogated and ventricular fibrillation did not occur (see FIG. 12). These data further confirm that the renin originates from cardiac mast cells because it disappeared from the coronary effluent when hearts were perfused with lodoxamide prior to induction of ischemia. Lodoxamide also prevented ventricular fibrillation.

FIGS. 11-14 also illustrate that ventricular fibrillation is initiated by the release of renin from cardiac mast cells and norepinephrine functions as the major final mediator of reperfusion arrhythmias. According to this scheme (FIG. 2), mast-cell-derived renin generates ANG I, which is next converted by ACE to ANG II near sympathetic nerves. ANG II then activates ANG II receptors (AT₁R) on nerve endings, thus potentiating carrier-mediated norepinephrine release, as the inventors have previously determined. Reid et al., Am J. Physiol. Heart Circ Physiol.286:H1448-H1454 (2004).

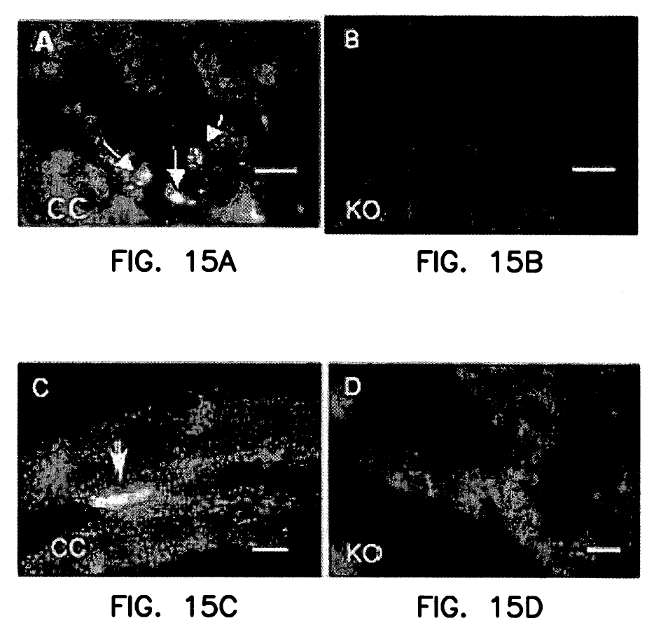

The finding that the duration of ventricular fibrillation varied as a function of norepinephrine overflow (FIG. 14) is consistent with the involvement of renin in ventricular fibrillation and related heart problems.

To determine the extent by which renin from mast cells can initiate a pathologic event, like, for instance, cardiac arrhythmias, hearts from mast-cell deficient mice (WBB6F₁-W/W^{v} mice; Jackson laboratory, stock #100410)(KO) and their congenic controls WBB6F1-⁺/⁺ (CC) were studied. These c-Kit knockout mice are 90% mast cell deficient. Kitamura, Y., Go, S., Hatanaka, K., Decrease of mast cells in W/Wv mice and their increase by bone marrow transplantation, Blood. August 52: 447-452 (1978). Hearts were screened from KO and CC mice for mast cells using the anti-renin antibody. FIG. 15 shows representative images of sections from CC and KO mouse hearts. Only CC hearts display anti-renin antibody immunoreactivity in the mast cells. There was no staining in nerves or myocytes.

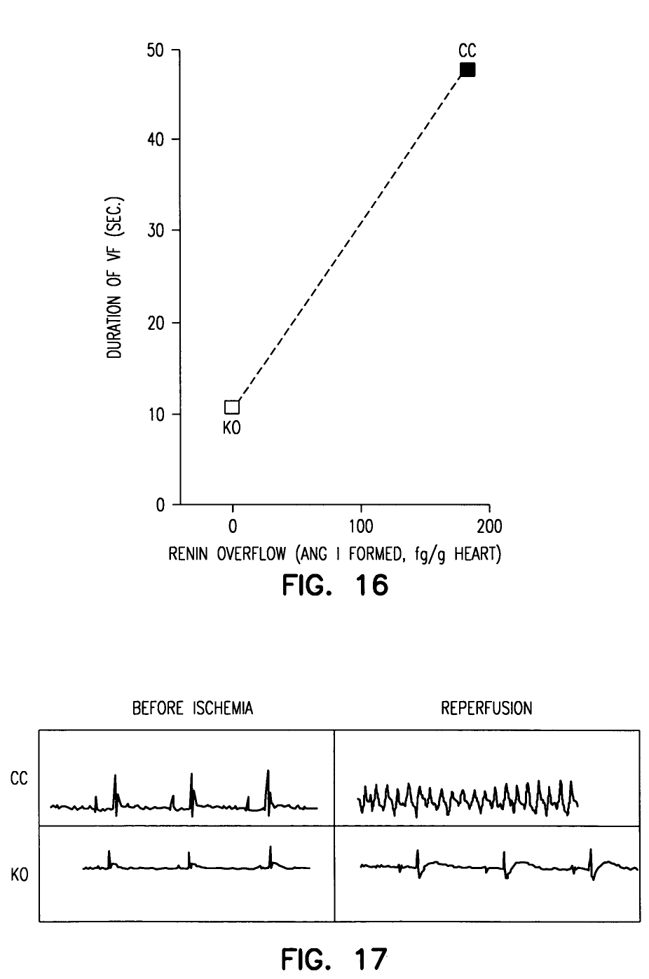

Having established the lack of mast cells in KO mouse hearts, hearts from these mice were isolated, suspended in a Langendorff apparatus and subjected to 30-min. stop-flow global ischemia, followed by 45-min. reperfusion (see methods described above). Surface ECG was recorded and renin overflow into the coronary effluent was measured. As seen in FIG. 16, no renin overflow occurred during reperfusion of mast-cell deficient hearts, and ventricular fibrillation only lasted about 10 sec. In contrast, CC hearts released a considerable amount of renin and fibrillated for about 50 sec.

FIG. 17 shows representative ECG recordings from one CC (top) and one KO (bottom) spontaneously beating heart subjected to 30-min global ischemia followed by 45-min reperfusion ex vivo in a Langendorff apparatus. The left tracings were recorded at the end of the equilibration period prior to ischemia, and show normal sinus rhythm in both CC and KO mouse hearts. The ECG tracings on the right were from the respective heart during reperfusion, showing ventricular fibrillation in the CC heart and an absence of ventricular fibrillation in the KO heart. These findings indicate that ischemia elicits the release of mast-cell-derived renin that activates local RAS exacerbating reperfusion arrhythmias. As illustrated, the arrhythmogenic function of ANG II is locally derived.

### EXAMPLE 7

### Na⁺/H⁺ Exchangers are a Pivotal Trigger for Carrier-mediated Norepinephrine Release

This Example provides data showing that angiotensin II potentiates carrier-mediated norepinephrine release in myocardial ischemia by a direct action on Na⁺/H⁺ exchangers in cardiac sympathetic nerve endings.

These experiments employed cardiac sympathetic nerve endings isolated from the guinea-pig heart and human neuroblastoma SH-SY5Y cells transfected with angiotensin II receptor (AT_{1A}R)(McDonald et al., Neurosci. Lett. 199: 115-118, 1995). SH-SY5Y cells are regarded as an optimal nerve ending model (Vaughan et al., Gen. Pharmacol. 26: 1191-1201, 1995).

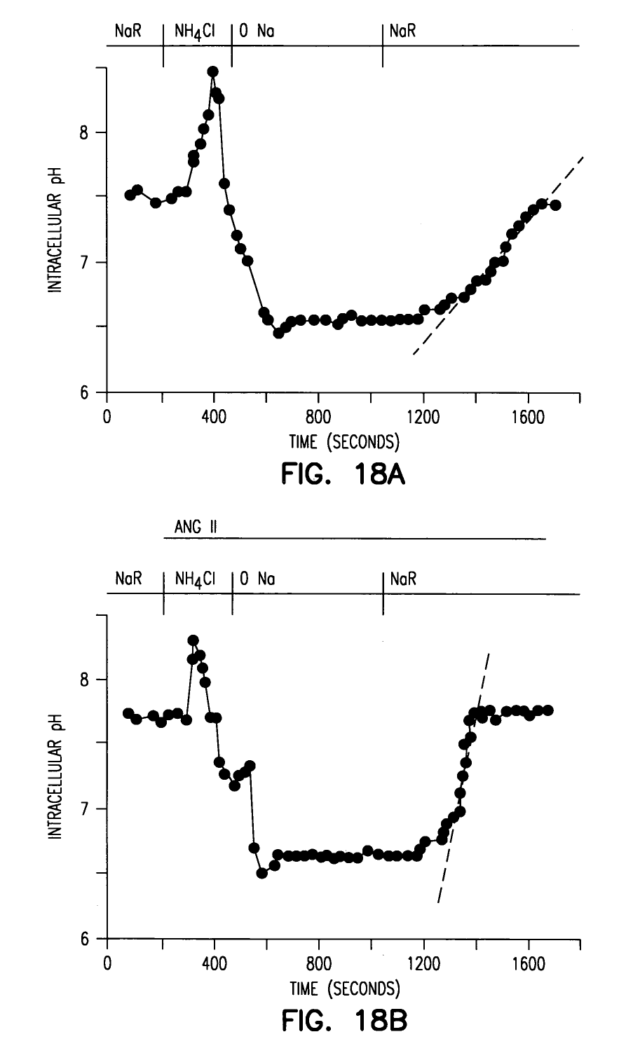

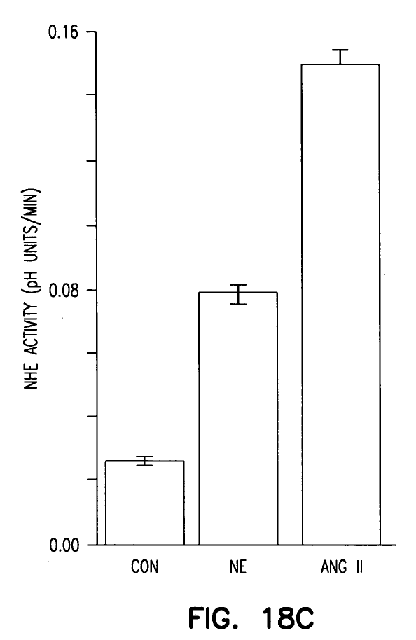

Na⁺/H⁺ exchanger activity was assayed by measuring the rate of Na⁺-dependent intracellular alkalinization in response to an acid load (NH₄Cl). An example of the “acid pulse protocol” used to show Na⁺/H⁺ exchanger activity in dye-loaded (BCECF-loaded) sympathetic nerve endings from guinea pig heart is shown in FIG. 18A. This protocol was performed under HEPES-buffered conditions. All of the solutions perfusing the tissue were osmotically balanced using N-methyl-D-glucamine substitution as previously described (Silver et al., Am. J. Physiol. Renal Physiol. 279: F195-F202, 2000; Silver et al., Am. J. Physiol. 275: F94- 102, 1998). As shown in the representative tracing in FIG. 18A, guinea-pig heart sympathetic nerve endings, attached to a cover slip and pre-loaded with BCECF to monitor intracellular pH, were initially bathed in a balanced salt solution (Na Ringer's solution). Exposing sympathetic nerve endings to an acute pulse of 10 mM NH₄Cl, by addition and removal of NH₄Cl, resulted in intracellular acidosis with intracellular pH decreasing 1 pH unit from 7.5 to 6.5. Intracellular pH did not recover in the Na-free solution. Re-addition of Na resulted in Na⁺-dependent intracellular alkalinization.

The slope of this alkalinization of Na⁺/H⁺ exchanger activity, represented by the dashed line in FIG. 18A, was 0.04 pH units/min. In sympathetic nerve endings exposed to angiotensin II (10 nM) as shown in FIG. 18B, the rate of Na⁺-dependent intracellular alkalinization was significantly greater than in control (0.19 pH units/min). Mean Na⁺/H⁺ exchanger activities measured in cardiac sympathetic nerve endings in this pilot study are compared in FIG. 18C. It is evident that superfusion with angiotensin II (10 nM) causes an increase in Na⁺/H⁺ exchanger activity to about 6-fold that observed for control, as compared with norepinephrine (1 μM), which increased Na⁺/H⁺ exchanger activity about 3-fold over control.

Similarly, Na⁺/H⁺ exchanger activity was measured in response to angiotensin II at the individual cell level in BCECF-loaded SH-SY5Y-AT_{1A} cells. Angiotensin II (10 nM) elicited a 2.5-fold increase in the Na⁺-dependent intracellular pH recovery rate in response to an acid load (FIG. 19). Exposing the cells to BAPTA-AM (10 μM) prior to angiotensin II, prevented the angiotensin II-induced increase in Na⁺/H⁺ exchanger activity. These results suggest that angiotensin II enhances Na⁺/H⁺ exchanger activity via a Ca²⁺-dependent intracellular signaling pathway.

These results indicate that angiotensin II is a potent stimulant of Na⁺/H⁺ exchanger activity in cardiac sympathetic nerve endings.

### EXAMPLE 8

### Angiotensin II Evokes Norepinephrine Release from Cardiac Sympathetic Nerve Endings by a Carrier-Mediated Mechanism

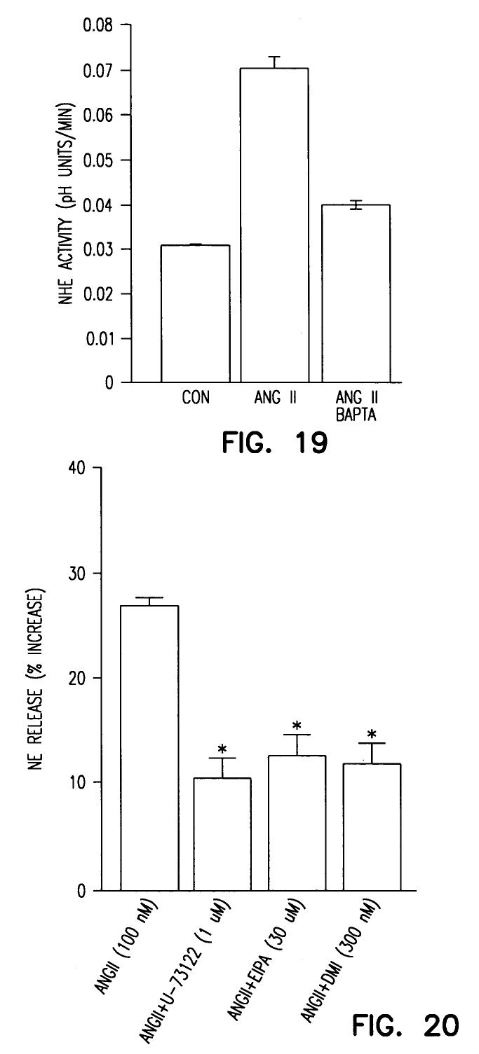

As shown in FIG. 20, the administration of angiotensin II to sympathetic nerve endings isolated from guinea-pig hearts resulted in a 27% increase in norepinephrine release above basal level. In the presence of the Na⁺/H⁺ exchanger inhibitor 5-(N-ethyl-N-isopropyl)-amiloride (EIPA, 30 μM), norepinephrine release was decreased by about 50%. Pretreatment with the norepinephrine transporter inhibitor DMI (300 nM) also decreased the angiotensin II-induced norepinephrine release by about 50%. These findings show that both Na⁺/H⁺ exchanger and norepinephrine transporter are essential for the elicitation of norepinephrine release by angiotensin II, indicating that a carrier-mediated mechanism must be playing a major role in this process.

The release of norepinephrine from cardiac sympathetic nerve endings is initiated by activation of angiotensin type 1 receptors (FIG. 8). Angiotensin type 1 receptors are known to be coupled to Gq/11 and PLC (De Gasparo et al., Pharmacol Rev 52: 415-472, 2000). Hence, the next experiments were designed to assess whether PLC plays a role in the release of norepinephrine by angiotensin II from cardiac sympathetic nerve endings. Tests were conducted to ascertain whether angiotensin II-evoked norepinephrine was affected by the PLC inhibitor U-73122 (1 μM) (Yule et al., J. Biol. Chem. 267: 13830-13835, 1992; Bleasdale et al., J. Pharmacol. Exp. Ther. 255: 756-768, 1990). As shown in FIG. 20, in the presence of U-73122, the magnitude of the angiotensin II-evoked norepinephrine release was reduced by about 60%. This indicates that PLC stimulation is part of the signaling pathway of the angiotensin type 1 receptor-mediated norepinephrine release by angiotensin II in cardiac sympathetic nerve endings.

The next series of experiments were used to outline the major signaling steps in angiotensin Il-evoked release of norepinephrine from sympathetic nerve endings. In particular, experiments were designed to ascertain whether stimulation of PLC might result in an increase in intracellular calcium via IP3 generation. Intracellular calcium (Cai) was measured at the individual cell level using Fura-2 loaded SH-SY5Y-AT_{1A} cells (McDonald et al., Neurosci. Lett. 199: 115-118, 1995; Silver et al., Proc. Natl. Acad. Sci. USA 99: 501-506, 2002). These SH-SY5Y-AT_{1A} cells were acutely exposed to angiotensin II (10 nM).

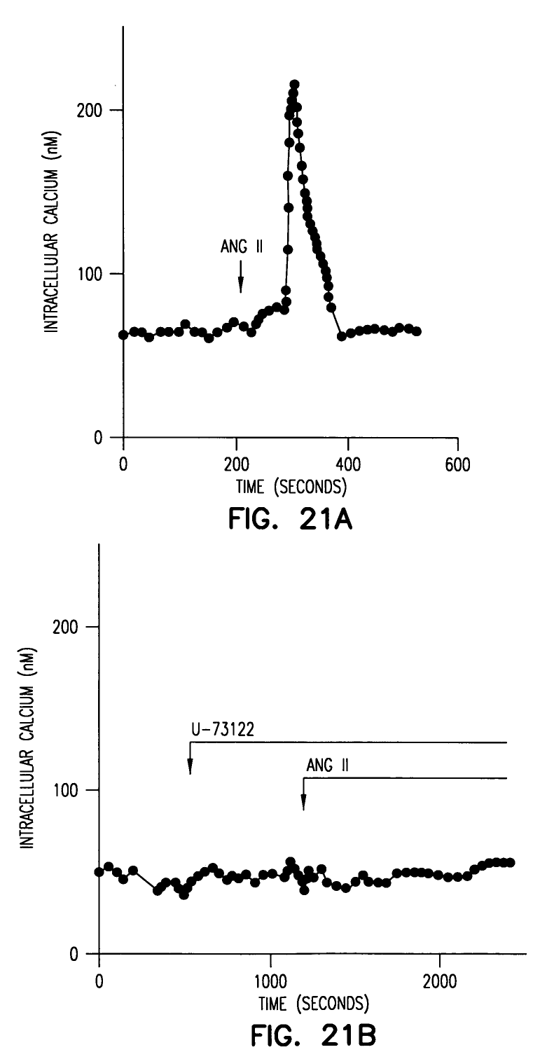

As shown in FIG. 21A, exposure to angiotensin II resulted in an abrupt increase in intracellular calcium from about 60 nM to 220 nM, which then rapidly returned to basal level. As discussed above, PLC activation may be an early signaling step in mediating this angiotensin II-induced Ca²⁺ transient. The PLC inhibitor U-73122 (1 μM) was used to verify this hypothesis. FIG. 21B is a representative trace from one Fura-2 loaded SH-SY5Y-ATIA cell pre-exposed to U-73122. As shown in FIG. 21 B, inhibition of PLC with this compound prevented transient changes in angiotensin II-induced intracellular calcium concentrations.

These findings indicate that inhibiting PLC prevents the angiotensin II-induced rise in intracellular calcium. This is consistent with the postulate that a PLC-dependent signaling pathway, resulting in a transient change in intracellular calcium concentration, is involved in angiotensin II-evoked norepinephrine release from cardiac sympathetic nerve endings.

PLC stimulation results not only in the generation of IP₃, but also in the release of diacylglycerol (DAG), which is involved in PKC activation (Berridge, Nature 361: 315-325, 1993.). PKC stimulation could lead to Na⁺/H⁺ exchanger activation by phosphorylation (Aviv et al., Am. J. Hypertens. 9: 703-707, 1996; Wakabayashi et al., Physiol. Rev. 77: 51-74, 1997) and, thus, to carrier-mediated norepinephrine release. This hypothesis was tested by ascertaining whether protein kinase C (PKC) activation using phorbol ester (PMA) would enhance the angiotensin II-induced release of N-methyl-4-phenylpyridinium (MPP⁺), a norepinephrine transporter substrate.

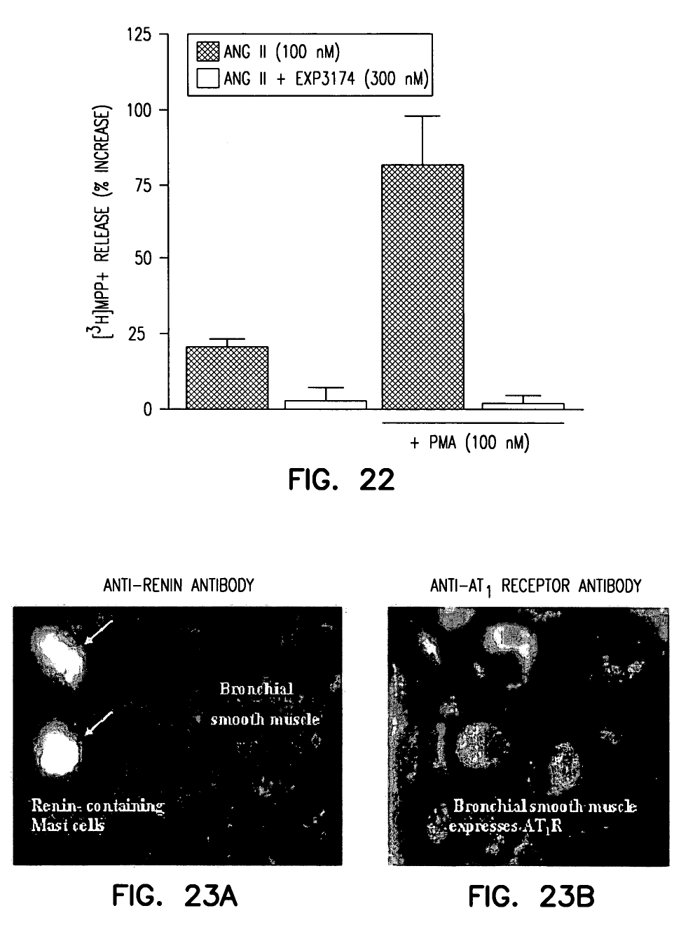

FIG. 22 shows that stimulation of PKC with a phorbol ester enhances the angiotensin II-induced release of MPP+from SH-SY5Y-AT_{1A} cells. MPP+was chosen for this particular experiment because it is an optimal norepinephrine transporter substrate (Smith et al., J. Pharmacol. Exp. Ther. 291: 456-463, 1999). Cells were pre-loaded with [³H]MPP⁺ and then incubated for 10 min with the selective angiotensin type 1 receptor antagonist EXP3174 (300 nM), followed by an 8-min incubation with or without the phorbol ester phorbol 12-myristate 13-acetate (PMA). Release of MPP⁺ was initiated by replacement of the incubation buffer with one containing either angiotensin II or angiotensin II+EXP3174. Efflux was arrested after 10-min exposure to angiotensin II. The findings indicate that angiotensin II causes a 20% increase in MPP⁺ release above basal levels. Moreover, PKC activation by PMA caused a 3-fold increase in the releasing effect of angiotensin II. Blockade of angiotensin type 1 receptor with EXP3174 completely prevented the releasing effect of angiotensin II, both in the presence and absence of PMA.

These experiments demonstrate that PKC activation greatly potentiates the effect of angiotensin II on the norepinephrine transporter in these neuroblastoma cells, which are viewed as a most favorable model of sympathetic neuron (McDonald et al., Neurosci. Lett. 199: 115-118, 1995; Vaughan et al., Gen. Pharmacol. 26: 1191-1201, 1995). Because norepinephrine release from cardiac sympathetic nerve endings appears to be a carrier-mediated process involving activation of Na⁺/H⁺ exchanger and reversal of the norepinephrine transporter in an outward direction (FIG. 20), the evidence presented in FIG. 22 suggests that the PMA-induced enhancement of norepinephrine release by angiotensin II is likely due to a PKC-mediated activation of Na⁺/H⁺ exchanger. This is consistent with the signaling pathways identified herein that mediate the effects of angiotensin II on Na⁺/H⁺ exchanger activity and associated norepinephrine release in cardiac sympathetic nerve endings.

### EXAMPLE 9

### Renin-Positive Mast Cells Exist in Lung Tissues

Sections cut from intact rat lung were screened for renin-like protein by indirect immunofluorescence microscopy with the polyclonal rabbit anti-renin BR1. Cryostat sections (10 μm) of paraformaldehyde-fixed rat lung were incubated either with the anti-renin BRI (1:500) or anti-AT₁R antibodies followed fluorescein-conjugated anti-rabbit IgG (1:500) antibodies.

FIG. 23 shows that renin-positive mast cells are present in a section of rat lung. The mast cells are apposed to the bronchiole (FIG. 23B).

When renin is released from the mast cells resulting in local production of angiotensin, this angiotensin can bind to the angiotensin receptors (AT₁R) found on the smooth muscle of bronchi (FIG. 23A-B) leading to smooth muscle contraction, as shown in the graph in FIG. 24.

### EXAMPLE 10

### Renin-Positive Cells Exist in Gut Tissues and Liver

Sections cut from intact rat gut and liver tissues were screened for renin by indirect immunofluorescence microscopy with the polyclonal rabbit anti-renin BR1. Cryostat sections (10 μm) of paraformaldehyde-fixed rat tissues were incubated with the anti-renin BRI (1:500) antibodies followed by fluorescein-conjugated anti-rabbit IgG (1:500) antibodies.

FIG. 25 shows that renin-positive cells are present in a section of rat gut, ileum, and liver.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A method for treating or preventing a condition in which renin is overly active in a patient comprising administering to the patient a composition that can inhibit renin release from a mast cell.

2. The method of claim 1, wherein the condition is associated with increased numbers of mast cells.

3. The method of claim 1, wherein the condition wherein the condition leads to increased angiotensin formation.

4. The method of claim 1, wherein the condition wherein the condition is associated with an inflammation.

5. The method of claim 1, wherein the condition is myocardial ischemia.

6. The method of claim 1, wherein the condition is congestive heart failure, atherosclerotic coronary artery disease, or chronic obstructive pulmonary disease.

7. The method of claim 1, wherein the condition is chronic obstructive pulmonary disease, Cor pulmonale, bronchiectasis, acute respiratory distress syndrome, bronchiolitis obliterans-organizing pneumonia, cystic fibrosis, interstitial lung diseases, silicosis, sarcoidosis, lung cancer, tuberculosis, gastritis, peptic ulcer, hepatocellular carcinoma, ulcerative colitis, Crohn's disease, liver cirrhosis, hepatitis, pancreatitis, atherosclerosis, myocardial infarction, congenital heart disease, myocarditis, cardiomyopathy, brain infarction, diabetes, thyroiditis, osteoporosis, glomerulonephritis, nephropathy, multiple sclerosis, rheumatoid arthritis, osteoarthritis, rheumatic arthritis congestive heart failure, cardiac hypertrophy, hypertension, cardiomyopathy, asthma, endometriosis, brain infarction, liver fibrosis, lung fibrosis, kidney fibrosis, heart fibrosis, skin fibrosis, interstitial cystitis, pancreatic cancer, or cardiomyopathy.

8. The method of claim 1, wherein the composition that can inhibit renin release from a mast cell comprises lodoxamide, cromolyn sodium, nedocromil, nicardipine, barnidipine, YC-114, elgodipine, niguldipine and R(−)-niguldipine, a dihydropyridine, nicardipine or nifedipine.

9. The method of claim 1, wherein the composition further comprises an ACE inhibitor.

10. The method of claim 9, wherein the ACE inhibitor is enalaprilat.

11. The method of claim 1, wherein the composition further comprises an angiotensin type 1 receptor inhibitor.

12. The method of claim 11, wherein the angiotensin type 1 receptor inhibitor is valsartan, olmesartan, candesartan, irbesartan, losartan or telmisartan.

13. The method of claim 1, wherein the composition further comprises an agent that can inhibit sodium/hydrogen exchange type-1 (NHE-1) transport systems.

14. The method of claim 1, wherein the composition is administered locally into cardiac, vascular, lung, liver, cervical, intestinal, muscle, pancreatic, brain, kidney or skin tissues.

15. The method of claim 1, wherein the composition is administered locally via a sustained release implant.

16. The method of claim 1, wherein the composition is administered locally via a stent.

17. A method for treating or preventing a condition in which renin is overly active in a patient comprising locally administering to an affected organ in the patient a composition that can inhibit renin expression or activity.

18. The method of claim 17, wherein the condition is associated with increased numbers of mast cells.

19. The method of claim 17, wherein the condition wherein the condition leads to increased angiotensin formation.

20. The method of claim 17, wherein the condition wherein the condition is associated with an inflammation.

21. The method of claim 17, wherein the condition is myocardial ischemia.

22. The method of claim 17, wherein the condition is congestive heart failure, atherosclerotic coronary artery disease, or chronic obstructive pulmonary disease.

23. The method of claim 17, wherein the condition is chronic obstructive pulmonary disease, Cor pulmonale, bronchiectasis, acute respiratory distress syndrome, bronchiolitis obliterans-organizing pneumonia, cystic fibrosis, interstitial lung diseases, silicosis, sarcoidosis, lung cancer, tuberculosis, gastritis, peptic ulcer, hepatocellular carcinoma, ulcerative colitis, Crohn's disease, liver cirrhosis, hepatitis, pancreatitis, atherosclerosis, myocardial infarction, congenital heart disease, myocarditis, cardiomyopathy, brain infarction, diabetes, thyroiditis, osteoporosis, glomerulonephritis, nephropathy, multiple sclerosis, rheumatoid arthritis, osteoarthritis, rheumatic arthritis congestive heart failure, cardiac hypertrophy, hypertension, cardiomyopathy, asthma, endometriosis, brain infarction, liver fibrosis, lung fibrosis, kidney fibrosis, heart fibrosis, skin fibrosis, interstitial cystitis, pancreatic cancer, or cardiomyopathy.

24. The method of claim 17, wherein the composition that can inhibit renin activity comprises BILA2157, aliskiren, remikiren, ankiren or enalkiren.

25. The method of claim 17, wherein the composition that can inhibit renin activity comprises a peptide comprising any one of SEQ ID NO:3-52.

26. The method of claim 17, wherein the composition that can inhibit renin expression comprises a nucleic acid complementary to any one of SEQ ID NO:53-54.

27. The method of claim 17, wherein the composition that can inhibit renin expression comprises a nucleic acid complementary to any one of SEQ ID NO:55-57.

28. The method of claim 17, wherein the composition that can inhibit renin expression comprises a nucleic acid comprising any one of SEQ ID NO:58-61.

29. The method of claim 17, wherein the composition further comprises an ACE inhibitor.

30. The method of claim 28, wherein the ACE inhibitor is enalaprilat.

31. The method of claim 17, wherein the composition further comprises an angiotensin type 1 receptor inhibitor.

32. The method of claim 31, wherein the angiotensin type 1 receptor inhibitor is valsartan, olmesartan, candesartan, irbesartan, losartan or telmisartan.

33. The method of claim 17, wherein the composition further comprises an agent that can inhibit sodium/hydrogen exchange type-1 (NHE-1) transport systems.

34. The method of claim 17, wherein the composition is administered locally into cardiac, vascular, lung, liver, cervical, intestinal, muscle, pancreatic, brain, kidney or skin tissues.

35. The method of claim 17, wherein the composition is administered locally via a sustained release implant.

36. The method of claim 17, wherein the composition is administered locally via a stent.

37. A composition comprising a carrier and an siRNA that can inhibit renin RNA function, wherein the composition is formulated for localized delivery.

38. The composition of claim 37, wherein the siRNA is complementary to a nucleic acid sequence comprising SEQ ID NO:53 or 54.

39. The composition of claim 37, wherein the siRNA is complementary to any one of SEQ ID NO:55-57.

40. The composition of claim 37, wherein siRNA comprises a nucleic acid comprising any one of SEQ ID NO:58-61.

41. The composition of claim 37, wherein the composition is formulated for local administration to heart, vascular, lung, bladder, skin, liver, kidney, pancreas, or gastrointestinal tissues.

42. An siRNA comprising any one of SEQ ID NO:58-61, wherein the siRNA can inhibit renin RNA function.

43. A composition comprising a carrier and an inhibitor of renin, wherein the composition is formulated for localized delivery to a tissue.

44. The composition of claim 43, wherein the composition is formulated for local administration to heart, vascular, lung, bladder, skin, liver, kidney, pancreas, or gastrointestinal tissues.

45. A composition comprising a carrier and an inhibitor of mast cell degranulation, wherein the composition is formulated for inhibiting renin release from mast cells at localized sites in a tissue.

46. The composition of claim 45, wherein the composition is formulated for local administration to heart, vascular, lung, bladder, skin, liver, kidney, pancreas, or gastrointestinal tissues.
